(19) United States
(12) Patent Application Publication
(10) Pub. No.: US 2022/0290122 A1
(43) Pub. Date: Sep. 15, 2022

(54) METHODS AND AGENTS FOR MODULATING MITOCHONDRIAL NAD LEVELS

(71) Applicant: Whitehead Institute for Biomedical Research, Cambridge, MA (US)

(72) Inventors: Nora Kory, Allston, MA (US); David M. Sabatini, Cambridge, MA (US)

(21) Appl. No.: 17/432,479

(22) PCT Filed: Feb. 19, 2020
(86) PCT No.: PCT/US20/18914

§ 371 Date: Aug. 19, 2021

Related U.S. Application Data
(60) Provisional application No. 62/807,730, filed on Feb. 19, 2019.

(51) Int. Cl. C12N 15/10; G01N 33/50; A61P 35/00

(52) U.S. Cl. CPC C12N 15/1006; G01N 33/5011; A61P 35/00; C12Q 2600/178

(57) ABSTRACT

Disclosed herein are methods and compositions for modulating MCART1 expression and activity to treat diseases such as cancer and age related conditions. Also disclosed are methods of screening for MCART1 modulation agents.

### RELATED APPLICATIONS

This application claims the benefit of U.S. Provisional Application No. 62/807,730, filed Feb. 19, 2019. The entire teachings of the above application are incorporated herein by reference.

### GOVERNMENT SUPPORT

This invention was made with government support under Grant Nos. CA241332, R01 CA103866, R01 CA129105, and R37 AI47389 awarded by the National Institutes of Health. The government has certain rights in the invention.

### BACKGROUND OF THE INVENTION

Nicotinamide adenine dinucleotide (NAD) is an essential co-factor required for redox reactions of nearly all metabolic pathways including glycolysis and tricarboxylic acid (TCA) cycle. A decline in NAD levels has been associated with aging and administration of NAD is currently being tested as a measure to prolong life-span and prevent age-associated diseases in the clinic. NAD is especially critical in mitochondrial metabolism and directly involved in the generation of ATP, but how NAD is imported into mitochondria in mammalian cells remains unknown. Inhibiting metabolic pathways, such as via inhibition of complex I of the electron transport chain, is currently being investigated as a target for cancer therapy.

### SUMMARY OF THE INVENTION

It is disclosed herein that MCART1, an uncharacterized member of the SLC25 family of mitochondrial carriers, is co-essential with a cluster of mitochondrial genes functioning in the electron transport chain (ETC), its assembly or synthesis. Cells lacking MCART1 are defective in mitochondrial respiration, depend more on generation of ATP by glycolysis and have perturbed levels of TCA cycle intermediates. Levels of respiratory chain complexes including mitochondrially translated subunits are unchanged in cells lacking MCART1 as are mtDNA and mitochondrial mass, ruling out that a general mitochondrial defect is responsible for the phenotype of MCART1-null cells. Metabolite profiling of mitochondria revealed an absence of NAD+ and its reduced form, NADH, specifically from mitochondria in cells lacking MCART1. As a result, the activity of complex I of the ETC, NADH:ubiquinone oxidoreductase, to which NADH donates its electrons, is absent in MCART1-null cells while the function of the other complexes is unaffected. The respiratory defect of cells lacking MCART1 is rescued by expression of a known yeast mitochondrial NAD+ transporter, NDT1. Thus, MCART1 functions as a mitochondrial transporter for NAD+ or its precursors in mammalian cells.

The identification of MCART1 has implications for a wide spectrum of disorders. MCART1 deficiency is useful as a model for mitochondrial disease due to complex I deficiency. Metabolite or genetic interventions that are able to bypass MCART1 function are useful strategies to treat mitochondrial disease. Boosting cellular NAD levels has been shown to delay the onset of aging and age-related diseases. Thus, increasing cellular NAD levels by increasing MCART1 levels or activity can be used to promote longevity. Further, complex I inhibitors such as Rotenone have been shown to have anti-cancer effects. Thus, decreasing MCART1 expression or activity can be used for anti-cancer therapies.

Some aspects of the disclosure are related to a method of modulating or stabilizing a Nicotinamide Adenine Dinucleotide (NAD) level in a mitochondria in a cell, comprising modulating the expression of MCART1 or the activity of a gene product of MCART1 in the cell. As used herein throughout the specification, NAD and a NAD level refers to NAD in its oxidized form (NAD+) or its reduced form (NADH), or both. In some embodiments of the methods and compositions disclosed throughout the specification, NAD levels refers to a precursor of NAD+ or NADH. In some embodiments, the level of NAD is stabilized. In some embodiments, the expression of MCART1 or the activity of a gene product of MCART1 is increased, thereby increasing the level of NAD in the mitochondria. In some embodiments, the expression of MCART1 or the activity of a gene product of MCART1 is decreased, thereby decreasing the level of NAD in the mitochondria. In some embodiments, the expression of MCART1 or the activity of a gene product of MCART1 is modulated by contacting the cell with an agent. In some embodiments, the agent comprises a peptide, nucleic acid, small molecule, or hybrid thereof.

Some aspects of the disclosure are related to a method of treating or preventing a disease or disorder associated with an aberrant level of NAD in mitochondria of a subject, comprising administering to the subject an agent that modulates the expression of MCART1 or the activity of a gene product of MCART1. In some embodiments, administration of the agent stabilizes the level of NAD in mitochondria of the subject. In some embodiments, administration of the agent increases the level of NAD in mitochondria of the subject. In some embodiments, administration of the agent decreases the level of NAD in mitochondria of the subject. In some embodiments, the agent comprises a peptide, nucleic acid, small molecule, or hybrid thereof. In some embodiments, the disease or disorder is a mitochondrial disease or disorder (e.g., a complex I deficiency), a metabolic disease or disorder, a cardiovascular disease or disorder, a muscular disease or disorder, a neurological disease or disorder, a disease or disorder associated with fatigue, or a disease or disorder associated with aging.

Some aspects of the disclosure are related to a method of treating or preventing a disease or disorder associated with aging in a subject in need thereof, comprising administering to the subject an agent that modulates the expression of MCART1 or the activity of a gene product of MCART1 in the subject. In some embodiments, the disease or disorder associated with aging is a cardiovascular disease or disorder, a neurological disease or disorder, a metabolic disease or disorder, or a muscular disease or disorder. In some embodiments, administration of the agent stabilizes or increases the level of NAD in mitochondria of the subject. In some embodiments, the method further comprises administration of a second agent that increases cytoplasmic NAD levels when administered to the subject. In some embodiments, the second agent is nicotinamide riboside.

Some aspects of the disclosure are related to a method of retaining or increasing exercise capacity or reducing fatigue in a subject in need thereof, comprising administering to the subject an agent that modulates the expression of MCART1 or the activity of a gene product of MCART1. In some embodiments, the subject has reduced exercise capacity or increased fatigue due to aging. In some embodiments, administration of the agent stabilizes or increases the level of NAD in mitochondria of the subject. In some embodiments, the method further comprises administration of a second agent that increases cytoplasmic NAD levels when administered to the subject. In some embodiments, the second agent is nicotinamide riboside.

Some aspects of the disclosure are related to a method of inhibiting the growth or viability of a cancer cell, comprising contacting the cancer cell with an agent that reduces the expression of MCART1 or the activity of a gene product of MCART1. In some embodiments, the method further comprises contacting the cancer cell with a second agent having anti-cancer activity. In some embodiments, the second agent inhibits the expression or activity of Complex I (i.e., respiratory complex I, EC 1.6.5.3). In some embodiments, the second agent is an amiloride, an amiloride derivative, or a biguanide derivative. In some embodiments, the cancer cell is contacted in vivo (e.g., the agent(s) are administered to a subject in need thereof).

Some aspects of the disclosure are related to a composition comprising an agent that that modulates the expression of MCART1 or the activity of a gene product of MCART1 when administered to a subject. In some embodiments, the agent increases the expression of MCART1 or the activity of a gene product of MCART1. In some embodiments, the composition further comprises a second agent that increases cytoplasmic NAD levels when administered to the subject. In some embodiments, the second agent is nicotinamide riboside.

In some embodiments, the agent decreases the expression of MCART1 or the activity of a gene product of MCART1. In some embodiments, the composition further comprises an anti-cancer agent.

Some aspects of the disclosure are related to a method of identifying a candidate agent that modulates the expression of MCART1 or the activity of a gene product of MCART1 in a mitochondria, comprising contacting the mitochondria with a test agent, measuring a level of NAD in the mitochondria, and identifying the agent as an inhibitor of expression of MCART1 or the activity of a gene product of MCART1 if the level of NAD or a precursor thereof in the mitochondria is lower than a reference level, or identifying the test agent as an agent that increases expression of MCART1 or the activity of a gene product of MCART1 if the level of NAD or a precursor thereof in the mitochondria is higher than a reference level, wherein the reference level is the level of NAD or a precursor thereof in mitochondria under equivalent conditions but not exposed to the test agent.

### BRIEF DESCRIPTION OF THE DRAWINGS

These and other characteristics of the present invention will be more fully understood by reference to the following detailed description in conjunction with the attached drawings. The patent or application file contains at least one drawing executed in color. Copies of this patent or patent application publication with color drawings will be provided by the Office upon request and payment of the necessary fee.

FIGS. 1A-1I show MCART1 (SLC25A51) is an inner mitochondrial membrane solute carrier required for ETC function. (FIG. 1A) The respiratory complex I core subunit NDUFS1 is co-essential with a cluster of genes involved in electron transport chain function. Gene essentiality scores from the Achilles dataset across 341 different cell lines (8) were correlated to identify genes co-essential with NDUFS1 as described (7). Genes with a gene ontology annotation of electron transport chain or mitochondrial gene expression are highlighted in red. (FIG. 1B) MCART1 is co-essential with genes involved in mitochondrial respiration. Gene ontology annotation of the top 20 (left panel) and top 100 (right panel) highest correlated genes with MCART1. ETC—electron transport chain; Mt—mitochondrial; Fe—S—iron-sulfur. (FIG. 1C) Barcode plot displays enrichment of components of the electron transport chain, tricarboxylic acid (TCA) cycle and mitochondrial DNA replication, but not of other mitochondrial processes. All genes from the Achilles dataset are plotted from lowest to highest correlating with MCART1 and genes of the depicted category are displayed by black lines. (FIG. 1D) Phylogenetic tree of the human SLC25 family of mitochondrial carriers, which includes MCART1 and the closely related MCART2 and MCART6 in red. The number of each family member and, if they have one, their alias is included. % Sequence identity of MCART1, 2 and 6 is shown. (FIG. 1E) Model of the predicted topology of MCART1 in the mitochondrial inner membrane. Transmembrane helices are indicated by numbers. IMS—intermembrane space. (FIG. 1F) Super-resolution microscopy confirms MCART1 localization to the inner membrane of mitochondria. Wild-type HeLa cells transiently expressing FLAG-MCART1 were processed for immunofluorescence detection of the FLAG epitope (magenta) and the mitochondrial inner membrane marker cytochrome c oxidase subunit 4 (COX4) (left panel, green) or the outer mitochondrial membrane marker Tom20 (right panel, green) and imaged by STED microscopy. Overlap of magenta and green channels is shown in white. Scale bar is 2 μm. Line profiles show fluorescent signals of each channel across mitochondria where marked by the dotted rectangles in images. (FIG. 1G) Loss of MCART1 decreases the oxygen consumption rate (OCR) of cells. Oxygen consumption rate was measured by Seahorse Extracellular Flux Analysis (mean±SD; n>13 technical replicates; ***P<0.001, ****P<0.0001). First three time points are basal respiration, subsequent time points are with serial injections of oligomycin, FCCP, and antimycin A/rotenone, respectively. (FIG. 1H) MCART 1 -null cells are unable to proliferate using galactose as the main carbon source. Proliferation of wild-type and MCART1-null Jurkat cells was assayed in RPMI containing glucose or galactose as indicated (mean±SD; n=3; ***P<0.001, ****P<0.0001). (FIG. 1I) Mitochondrial ATP production is strongly reduced relative to glycolytic ATP production upon MCART1 loss (mean±SD; n≥13 technical replicates; ****P<0.0001). Graphs were generated from data in the Seahorse experiment shown in FIG. 6A using the Seahorse Report Generator.

FIGS. 2A-2G show that loss of MCART1 causes loss of ETC (electron transport chain) complex I activity and defects in mitochondrial metabolism without affecting mitochondrial integrity. (FIG. 2A) Loss of MCART1 diminishes the activity of respiratory complex I, but not other complexes. Oxygen consumption rate (OCR) of indicated cells permeabilized and supplemented with ADP and complex I-IV substrates was measured by Seahorse extracellular flux analysis (mean±SD; n=3). Mal—malate; perm—permeabilizer; pyr—pyruvate; rot—rotenone. (FIG. 2B) Loss of MCART1 diminishes complex I-dependent state 3 respiration. Graph was calculated from the data in FIG. 2B (mean±SD; n=3). (FIG. 2C) Rotenone-sensitive NADH:ubiquinone activity in mitochondrial lysates is not dependent on MCART1 indicating complex I is functional in MCART1-null cells. (mean±SD; n=3; n.s.—not significant) (FIG. 2D) Several mitochondrial and mitochondria-derived metabolites are changed in cells lacking MCART1 compared to their wild-type counterparts. Metabolites were measured by LC-MS in extracts from indicated cells (mean±SD; n=3; **P<0.01). (α-KG—α-ketoglutarate; ser—serine; gly—glycine; AICAR—5-Aminoimidazole-4-carboxamide ribonucleotide). (FIG. 2E) Loss of MCART1 increases glucose consumption and lactate and malate excretion, and decreases pyruvate secretion. Medium metabolites were extracted after growing cells for 48 hours in RPMI media (mean±SD; n=3). Values are normalized to cell number. (FIG. 2F) Glutamine tracing scheme used to measure TCA cycle (The Citric Acid cycle) flux. (FIG. 2G) TCA cycle flux depends on MCART1. Isolated mitochondria were incubated with ¹³C₅, ¹⁵N₂-glutamine, malate and ADP, and the metabolites generated from labeled glutamine in the first (α-KG M+5, succinate/malate/citrate/cis-aconitate M+4) and second rounds (malate/citrate M+2) of the TCA cycle according to the tracing scheme in the left panel were detected by LC-MS. (α-KG—α-Ketoglutarate; cis-Acon—cis-aconitate).

FIGS. 3A-3C show that NAD⁺ and NAD are depleted in the mitochondria of MCART1-null cells. (FIG. 3A) NAD⁺ and NADH are the most depleted metabolites in mitochondria of MCART1-null cells. The log2 fold change of metabolites detected in mitochondria isolated from MCART1-null cells versus in mitochondria from null cells expressing MCART1 cDNA (mean; n=3). (FIG. 3B) Loss of MCART1 depletes NAD⁺ and NADH in mitochondria and reduces TCA cycle intermediates. Whole cell and mitochondrial (mito) metabolite levels in indicated cells were measured by LC-MS using the Mito-IP method, data from two independent experiments with three replicates each were combined (mean±SD; n>5). Asterisks denote statistically significant differences of MCART1-null samples with both wild-type cells and cells re-expressing the MCART1 cDNA (*P<0.05, **P<0.01, ***P<0.001, ****P<0.0001; PEP—phosphoenolpyruvate; α-KG—α-Ketoglutarate; cis-Acon—cis-aconitate; Glu—glutamate; Asp—aspartate). (FIG. 3C) MCART1-null cells depend on glycolytic enzymes and mitochondrial FAD/folate transporter. Top-scoring genes from the MCART1 synthetic lethality screen. Genes were ranked according to the differential gene score in MCART1-null versus control cells. (Mito—mitochondrial; TCA—tricarboxylic acid; FAD—flavin adenine dinucleotide).

FIGS. 4A-4G show that defects of MCART1-null cells are rescued by expression of a yeast mitochondrial NAD⁺ transporter but not by a predicted substrate-binding mutant of MCART1. (FIG. 4A) Schematic of the NAD salvage pathway. (Nam—nicotinamide; NMN—nicotinamide mononucleotide; NRK—nicotinamide riboside kinase; NAMPT—nicotinamide phosphoribosyltransferase; NMNAT—nicotinamide-nucleotide adenylyltransferase). (FIG. 4B) The yeast mitochondrial NAD⁺ transporter NDT1 but not close sequence homologs of MCART1 rescue mitochondrial respiration as determined by growth in galactose as the carbon source. Single-cell-derived knockout Jurkat cells were transduced with an empty vector (EV) or cDNAs of MCART1, the mitochondrial FAD/folate carrier (MFT) or yeast mitochondrial transporters. Asterisks denote statistically significant differences in proliferation in media containing galactose as the carbon source between the cells expressing the empty vector and the solute carrier homologs (mean±SD; n=3; **P<0.01, ***P<0.001, ****P<0.0001, n.s.—not significant; ODC—oxodicarboxylate carrier, GGC—GTP/GDP carrier). (FIG. 4C) The yeast mitochondrial NAD⁺ transporter NDT1 rescues complex I activity in MCART1-null cells (mean±SD; n>3; ****P<0.0001). (FIG. 4D) The yeast mitochondrial NAD⁺ transporter NDT1 rescues mitochondrial NAD levels in MCART1-null cells (mean±SD; n>3; ****P<0.0001). (FIG. 4E) Sequence alignment of MCART1 homologs and sequence comparison with the ADP/ATP carrier identify lysine residue 91 in transmembrane domain 2 as a potential substrate contact point. Arginines 182 and 278 are potential other substrate contact points. SEQ ID NOS: 4-11 are shown. (FIG. 4F) Mutation of lysine 91 to alanine abolishes the ability of MCART1 to rescue growth on galactose. MCART1-null cells infected with wild-type MCART1 cDNA serve as control cells. (mean±SD; n=3; ****P<0.0001). (FIG. 4G) MCART1 K91A does not rescue mitochondrial NAD⁺ and NADH levels. MCART1-null cells infected with wild-type MCART1 cDNA serve as control cells (mean±SD; n=4; ***P<0.001, ****P<0.0001).

FIGS. 5A-5N show further evidence that MCART1 (SLC25A51) is an inner mitochondrial membrane solute carrier required for ETC function. (FIG. 5A) MCART1 correlates most strongly with NDUFB10 in the Achilles dataset. Plotted are gene essentiality scores for MCART1 and NDUFB10 over a panel of 341 cancer cell lines (8). (FIG. 5B) mRNA levels of human MCART homologs in commonly used cell lines and normal tissues. RPKM (Reads Per Kilobase Million) levels were extracted from the Cancer Cell Line Encyclopedia (39) and TPM (Transcripts Per Kilobase Million) levels were extracted from GTEx Portal V7 (mean±SD). (FIG. 5C) FLAG-tagged MCART1 localizes to mitochondria. Wild-type HeLa cells transiently expressing FLAG-MCART1 were processed for immunofluorescence detection of the FLAG epitope (cyan) and the mitochondrial inner membrane marker cytochrome c oxidase 4 (COX4) (magenta). The merged image shows the overlap of both channels in white. Scale bar is 10 μm. (FIG. 5D) Mitochondrial purification by Mito-IP shows endogenous MCART1 in the mitochondrial fraction. Shown are HA-immunoprecipitates and cell lysates from wild-type cells expressing an HA-mito tag or a control MYC-mito tag. IPs were validated by immunoblotting for the following proteins: CS—citrate synthase; VDAC1—voltage-dependent anion channel; CALR—calreticulin; GOLGA1—Golgin subfamily A member; LAMP2—lysosome-associated membrane glycoprotein; CAT—catalase; RPS6KB1—Ribosomal protein S6 kinase beta-1. (FIG. 5E) Next generation sequencing confirms homozygous 1 or 25 bp frame-shift deletions in the MCART1 open reading frame in two single-cell derived clones. MCART1 open reading frame shown is SEQ ID NO: 1 (FIG. 5F) Immunoblot showing loss of MCART1 in Jurkat single cell-derived clones. Lysates prepared from indicated knockout cells were equalized for total protein amount and analyzed by immunoblotting for the levels of the indicated proteins. (FIG. 5G) MCART1-null cells have a proliferation defect in full RPMI media (mean±SD; n=3). (FIG. 5H) Basal and maximal respiration, proton leak, ATP production and spare respiratory capacity are decreased in MCART1-null cells. Oxygen consumption rate was measured by Seahorse Extracellular Flux Analysis (mean±SD; n≥13 technical replicates; ***P<0.001, ****P<0.0001). Graphs were generated from data in the Seahorse experiment in FIG. 1I using the Seahorse Report Generator. (FIG. 5I) FLAG-tagged human MCART homologs localize to mitochondria. Wild-type HeLa cells transiently expressing FLAG-constructs were processed for immunofluorescence detection of the FLAG epitope (green) and the mitochondrial inner membrane marker COX4 (magenta). The merged image shows the overlap of both channels in white. Scale bar is 10 μm. (FIG. 5J) Immunoblot of MCART1-null cells expressing indicated N-terminally FLAG-tagged MCART cDNA constructs. Lysates prepared from indicated cell lines were equalized for total protein amounts and analyzed by immunoblotting for the FLAG-epitope or the levels of the indicated proteins. * indicates endogenous MCART1. ** indicates FLAG-tagged MCART1. (FIG. 5K) Human MCART2 but not MCART6 rescues the mitochondrial respiration defect of cells lacking MCART1. Single-cell-derived knockout Jurkat cells were transduced with an empty vector (EV) or cDNAs of human MCART homologs. Asterisks denote statistically significant differences in proliferation in media containing galactose as the carbon source and between the cells expressing the empty vector and the solute carrier homologs. Mean±SD; n=3; ****P<0.0001). (FIG. 5L) Within one experiment, the oxygen consumption rate (OCR) and proton efflux rate (PER) were measured by Seahorse extracellular flux analysis with sequential treatment of oligomycin and antimycin A/rotenone to calculate ATP production from glycolysis versus oxidative phosphorylation (mean±SD; n=5). (FIG. 5M) Supplementation of RPMI media of cells with metabolites known to alleviate mitochondrial dysfunction does not rescue the proliferation defect of MCART1-null cells (mean±SD; n=3; **P<0.01, ****P<0.0001). Pyr—pyruvate; HT—hypoxanthine-thymidine. (FIG. 5N) Supplementation of RPMI media of cells stably expressing the plasma membrane aspartate transporter SLC1A3 with 10 mM aspartate does not rescue the proliferation defect of MCART1-null cells (mean±SD; n=3; ***P<0.001, ****P<0.0001).

FIGS. 6A-6I show MCART1 loss does not affect mitochondria structure. (FIG. 6A) Loss of MCART1 does not affect mitochondrial morphology and length. Max intensity z-projections of confocal images of mitochondria visualized by MitoTracker Green (green in merged images) were used to measure mitochondrial length of indicated Jurkat cells. Nuclei were stained with Hoechst DNA stain (blue) (mean±SD; n>500; ****P<0.0001; n.s.—not significant). Scale bar is 5 μm. (FIG. 6B) Electron microscopy reveals normal cristae morphology in MCART1-null mitochondria. Two 3× magnified inset are shown. Scale bar is 200 nm. (FIG. 6C) Loss of MCART1 does not affect mtDNA content (mean±SD; n=3; ***P<0.001; n.s.—not significant). Mitochondrial DNA was quantified by qPCR and normalized to genomic DNA. (FIG. 6D) Loss of MCART1 does not affect mitochondrial mass per cell as determined by flow cytometry analysis of indicated Jurkat cells stained with MitoTracker Green. The histograms were normalized and smoothened (A.U.—arbitrary units). (FIG. 6E) Loss of MCART1 does not affect relative mitochondrial membrane potential as assessed by flow cytometry analysis of Jurkat cells stained with tetramethylrhodamine, methyl ester, and perchlorate (TMRM). Indicated cells were treated with 10 μM FCCP. The histograms were normalized and smoothened. (FIG. 6F) Loss of MCART1 does not affect protein levels of mitochondrially encoded (upper panel) and nuclear encoded (lower panel) mitochondrial respiratory chain complex subunits. Lysates prepared from indicated cells were equalized for total protein amounts and analyzed by immunoblotting for indicated proteins. (CI—complex I; CII—complex II; CIII—complex III; CIV—complex IV; mtDNA—mitochondrially encoded; nDNA—nuclear encoded; CS—citrate synthase). (FIG. 6G) MCART1-null cells are unable to oxidize exogenous substrate via respiratory complex I. Oxygen consumption rate (OCR) measured by Seahorse extracellular flux analysis of indicated cells permeabilized and supplemented with ADP and complex I substrates or ADP only (mean±SD; n=3). Mal—malate; perm—permeabilizer; pyr—pyruvate; rot—rotenone. (FIG. 6H) Complex I-dependent state 3 respiration is diminished in MCART1-null clone #2 as determined by Seahorse extracellular flux analysis (mean±SD; n=3; *P<0.05, ***P<0.001, ****P<0.0001; n.s.—not significant). (FIG. 6I) TCA cycle intermediates are still produced in MCART1-null cells at the whole cell level. Jurkat cells were incubated in RPMI media containing 2 mM ¹³C₅,¹⁵N₂-glutamine as the sole glutamine source for 2 hours before metabolites were extracted.

FIGS. 7A-7B show MCART1 depletion reduces NAD+ and NADH in mitochondria and modulates expression of metabolic genes. (FIG. 7A) NAD⁺ and NADH are depleted in mitochondria of MCART1-null clone #2 and TCA cycle intermediates are reduced. Whole cell and mitochondrial (mito) metabolite levels in indicated cells were measured by LC-MS using the Mito-IP method (mean±SD; n=4). Asterisks denote statistically significant differences of MCART1-null samples with both wild-type cells and cells re-expressing the MCART1 cDNA (*P<0.05, **P<0.01, ***P<0.001; PEP—phosphoenolpyruvate; α-KG—α-Ketoglutarate; cis-Acon—cis-aconitate; Glu—glutamate; Asp—aspartate). (FIG. 7B) Gene scores from MCART1-reexpressing control cells were plotted against those from MCART1-null cells. Genes with a differential score of <−2 or >2 are annotated as hits. (Mito—mitochondrial; TCA—tricarboxylic acid; FAD—flavin adenine dinucleotide).

FIGS. 8A-8I show further evidence that defects of MCART1-null cells are rescued by expression of a yeast mitochondrial NAD⁺ transporter but not by a predicted substrate-binding mutant of MCART1. (FIG. 8A) FLAG-tagged nicotinamide mononucleotide adenyltransferase (NMNAT) isoforms localize to the nucleus, the Golgi and mitochondria, respectively. Wild-type HeLa cells transiently expressing FLAG-constructs were processed for immunofluorescence detection of the FLAG epitope (green) and the mitochondrial inner membrane marker COX4 (magenta). The merged image shows the overlap of both channels in white. Scale bar is 10 μm. (FIG. 8B) Jurkat and K562 cells express MCART1, NMNAT1, and NMNAT3. mRNA levels were quantified by qPCR relative to β-ACTIN. Two primer pairs each were used for NMNAT1, 2 and 3. N.D.—not detected. (Mean±SD; n=3). (FIG. 8C) Human NMNAT3-null cells have no mitochondrial respiration defect. Single-cell-derived NMNAT3 knockout or control Jurkat cells were cultured in media containing glucose or galactose as the carbon source. (Mean±SD; n=3; n.s.—not significant). (FIG. 8D) Human NMNAT3-null cells have no mitochondrial respiration defect. A mitochondrial stress test was performed by Seahorse extracellular flux analysis on single-cell-derived NMNAT3 knockout or control Jurkat cells. (Mean±SD; n>10; n.s.—not significant). (FIG. 8E) Mitochondrial NAD levels do not depend on NMNAT3. (Mean±SD; n=4; n.s.—not significant). (FIG. 8F) Next generation sequencing confirms 20 bp and 5 bp or homozygous 5 bp frame-shift deletions in the NMNAT3 open reading frame in two single-cell derived clones. NMNAT3 open reading frame shown is SEQ ID NO: 2. (FIG. 8G) FLAG-tagged yeast NAD transporter NDT1, the 2-oxodicarboxylate transporters ODC1 and ODC2, the GTP/GDP transporter GGC1, human MFT and the MCART1 K91A mutant localize to mitochondria. NDT2 expression levels were not detectable. Wild-type HeLa cells transiently expressing FLAG-constructs were processed for immunofluorescence detection of the FLAG epitope (green) and the mitochondrial inner membrane marker COX4 (magenta). The merged image shows the overlap of both channels in white. Scale bar is 10 μm. (FIG. 8H) Immunoblot of MCART1-null cells expressing indicated N-terminally FLAG-tagged MCART1, MFT or yeast transporter cDNA constructs. Lysates prepared from indicated cells were equalized for total protein amounts and analyzed by immunoblotting for the FLAG-epitope or the levels of the indicated proteins. (FIG. 8I) Immunoblot of MCART1-null cells expressing indicated N-terminally FLAG-tagged wild-type or K91A mutant MCART1 cDNA constructs. Lysates prepared from indicated cells were equalized for total protein amounts and analyzed by immunoblotting for the FLAG-epitope or the levels of the indicated proteins.

### DETAILED DESCRIPTION OF THE INVENTION

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3rd ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N.J., 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange; 10th ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), as of May 1, 2010, ncbi.nlm.nih.gov/omim/ and in Online Mendelian Inheritance in Animals (OMIA), a database of genes, inherited disorders and traits in animal species (other than human and mouse), at omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

Some aspects of the disclosure are directed to a method of modulating or stabilizing a Nicotinamide Adenine Dinucleotide (NAD) level in a mitochondria in a cell. In some embodiments, the method comprises modulating the expression of MCART1 or the activity of a gene product of MCART1 in the cell. In some embodiments, the method comprises modulating the expression of MCART2 or the activity of a gene product of MCART2 in the cell. As used herein throughout the specification, NAD and a NAD level refers to NAD in its oxidized form (NAD+) or its reduced form (NADH), or both. In some embodiments of the methods and compositions disclosed throughout the specification, NAD levels refers to a precursor of NAD+ or NADH. In some embodiments, the level of NAD is stabilized (e.g., the rate of increase or decrease of an NAD level in a mitochondria is reduced or stopped, fluctuations in NAD levels are reduced or eliminated). In some embodiments, the expression of MCART1 or the activity of a gene product of MCART1 is increased, thereby increasing the level of NAD in the mitochondria. In some embodiments, the expression of MCART1 or the activity of a gene product of MCART1 is decreased, thereby decreasing the level of NAD in the mitochondria. In some embodiments, the expression of MCART2 or the activity of a gene product of MCART2 is increased, thereby increasing the level of NAD in the mitochondria.

MCART1 is also known as Solute Carrier Family 25 Member 51, Mitochondrial Carrier Triple Repeat Protein 1, Mitochondrial Carrier Triple Repeat 1, Solute Carrier Family 25, Member 51, CG7943, and SLC25A51. MCART1 UniprotKB is Q9H 1U9. In some embodiments, the gene product of MCART1 has the following sequence:

(SEQ ID NO: 3)
MMDSEAHEKR PPILTSSKQD ISPHITNVGE MKHYLCGCCA
AFNNVAITFP IQKVLFRQQL YGIKTRDAIL QLRRDGFRNL
YRGILPPLMQ KTTTLALMFG LYEDLSCLLH KHVSAPEFAT
SGVAAVLAGT TEAIFTPLER VQTLLQDHKH HDKFTNTYQA
FKALKCHGIG EYYRGLVPIL FRNGLSNVLF FGLRGPIKEH
LPTATTHSAH LVNDFICGGL LGAMLGFLFF PINVVKTRIQ
SQIGGEFQSF PKVFQKIWLE RDRKLINLFR GAHLNYHRSL
ISWGIINATY EFLLKVI.

In some embodiments, the gene product of MCART1 has a substitution at position 205 of SEQ ID NO: 3 of T to M. In some embodiments, the gene product of MCART1 is at least 70% at least 75%, at least 80%, at least 85%, at least 90%, at least 91%, at least 92%, at least 93%, at least 94%, at least 95%, at least 96%, at least 97%, at least 98%, or at least 99% homologous to SEQ ID NO: 3. In some embodiments, the gene product of MCART1 comprises amino acid sequences that are conserved between MCART1 and MCART2. In some embodiments, the MCART1 gene has gene ID: 92014. In some embodiments, the gene is at least 70% at least 75%, at least 80%, at least 85%, at least 90%, at least 91%, at least 92%, at least 93%, at least 94%, at least 95%, at least 96%, at least 97%, at least 98%, or at least 99% homologous to the sequence of gene ID: 92014. In some embodiments, the MCART1 gene sequence corresponds to cDNA for MCART1. In some embodiments, the MCART1 gene further comprises a vector (e.g., a coding vector).

MCART2 is also known as Solute Carrier Family 25 Member 52, Mitochondrial Carrier Triple Repeat Protein 2, Mitochondrial Carrier Triple Repeat 2, and SLC25A52. MCART2 UniProtKB is Q3SY17. MCART gene ID is 147407. In some embodiments, the gene product of MCART2 is at least 70% at least 75%, at least 80%, at least 85%, at least 90%, at least 91%, at least 92%, at least 93%, at least 94%, at least 95%, at least 96%, at least 97%, at least 98%, or at least 99% homologous to wild-type MCART2 protein. In some embodiments, the MCART2 gene is at least 70% at least 75%, at least 80%, at least 85%, at least 90%, at least 91%, at least 92%, at least 93%, at least 94%, at least 95%, at least 96%, at least 97%, at least 98%, or at least 99% homologous to wild-type MCART2 gene.

As used herein “modulating” (and verb forms thereof, such as “modulates”) means causing or facilitating a qualitative or quantitative change, alteration, or modification. Without limitation, such change may be an increase or decrease in a qualitative or quantitative aspect. For example, modulating transcription of a gene includes increasing or decreasing the rate or frequency of gene transcription.

In some embodiments, the expression of MCART1 or the activity of a gene product of MCART1 is increased by at least about 5%, 10%, 15%, 20%, 25%, 30%, 35%, 40%, 45%, 50%, 55%, 60%, 65%, 70%, 75%, 80%, 85%, 90%, 95%, 99% or more. In some embodiments, the expression of MCART1 or the activity of a gene product of MCART1 is increased by at least about 1.1 fold, at least 1.2 fold, 1.3 fold, at least 1.4 fold, at least 1.5 fold, at least 1.6 fold, at least 1.7 fold, at least 1.8 fold, at least 1.9 fold, at least 2 fold, at least 3 fold, at least 4 fold, at least 5 fold, at least 10 fold, at least 20 fold, at least 30 fold, at least 40 fold, at least 50 fold, or at least 100 fold, at least a 1,000 fold, at least 10,000 fold as compared to MCART1 that has not been modulated. In some embodiments, the expression of MCART1 or the activity of a gene product of MCART1 is reduced by at least about 5%, 10%, 15%, 20%, 25%, 30%, 35%, 40%, 45%, 50%, 55%, 60%, 65%, 70%, 75%, 80%, 85%, 90%, 95%, 99% or more.

In some embodiments, the expression or activity of MCART2 is increased by at least about 5%, 10%, 15%, 20%, 25%, 30%, 35%, 40%, 45%, 50%, 55%, 60%, 65%, 70%, 75%, 80%, 85%, 90%, 95%, 99% or more. In some embodiments, expression or activity of MCART2 is increased from substantially no expression or activity of MCART2.

In some embodiments, the expression of MCART1 or the activity of a gene product of MCART1 is modulated by contacting the cell with an agent (e.g., an effective amount of an agent). In some embodiments, the expression of MCART2 or the activity of a gene product of MCART2 is modulated by contacting the cell with an agent (e.g., an effective amount of an agent). “Agent” is used herein to refer to any substance, compound (e.g., molecule), supramolecular complex, material, or combination or mixture thereof. In some aspects, an agent can be represented by a chemical formula, chemical structure, or sequence. Example of agents, include, e.g., small molecules, polypeptides, nucleic acids (e.g., RNAi agents, antisense oligonucleotide, aptamers), lipids, polysaccharides, peptide mimetics, etc. In general, agents may be obtained using any suitable method known in the art. The ordinary skilled artisan will select an appropriate method based, e.g., on the nature of the agent. An agent may be at least partly purified. In some embodiments an agent may be provided as part of a composition, which may contain, e.g., a counter-ion, aqueous or non-aqueous diluent or carrier, buffer, preservative, or other ingredient, in addition to the agent, in various embodiments. In some embodiments an agent may be provided as a salt, ester, hydrate, or solvate. In some embodiments an agent is cell-permeable, e.g., within the range of typical agents that are taken up by cells and acts intracellularly, e.g., within mammalian cells. Certain compounds may exist in particular geometric or stereoisomeric forms. Such compounds, including cis- and trans-isomers, E- and Z-isomers, R- and S-enantiomers, diastereomers, (D)-isomers, (L)-isomers, (−)- and (+)-isomers, racemic mixtures thereof, and other mixtures thereof are encompassed by this disclosure in various embodiments unless otherwise indicated. Certain compounds may exist in a variety or protonation states, may have a variety of configurations, may exist as solvates (e.g., with water (i.e. hydrates) or common solvents) and/or may have different crystalline forms (e.g., polymorphs) or different tautomeric forms. Embodiments exhibiting such alternative protonation states, configurations, solvates, and forms are encompassed by the present disclosure where applicable.

An “analog” of a first agent refers to a second agent that is structurally and/or functionally similar to the first agent. A “structural analog” of a first agent is an analog that is structurally similar to the first agent. Unless otherwise specified, the term “analog” as used herein refers to a structural analog. A structural analog of an agent may have substantially similar physical, chemical, biological, and/or pharmacological propert(ies) as the agent or may differ in at least one physical, chemical, biological, or pharmacological property. In some embodiments at least one such property differs in a manner that renders the analog more suitable for a purpose of interest. In some embodiments a structural analog of an agent differs from the agent in that at least one atom, functional group, or substructure of the agent is replaced by a different atom, functional group, or substructure in the analog. In some embodiments, a structural analog of an agent differs from the agent in that at least one hydrogen or substituent present in the agent is replaced by a different moiety (e.g., a different substituent) in the analog.

In some embodiments, the agent is a nucleic acid. The term “nucleic acid” refers to polynucleotides such as deoxyribonucleic acid (DNA) and ribonucleic acid (RNA). The terms “nucleic acid” and “polynucleotide” are used interchangeably herein and should be understood to include double-stranded polynucleotides, single-stranded (such as sense or antisense) polynucleotides, and partially double-stranded polynucleotides. A nucleic acid often comprises standard nucleotides typically found in naturally occurring DNA or RNA (which can include modifications such as methylated nucleobases), joined by phosphodiester bonds. In some embodiments a nucleic acid may comprise one or more non-standard nucleotides, which may be naturally occurring or non-naturally occurring (i.e., artificial; not found in nature) in various embodiments and/or may contain a modified sugar or modified backbone linkage Nucleic acid modifications (e.g., base, sugar, and/or backbone modifications), non-standard nucleotides or nucleosides, etc., such as those known in the art as being useful in the context of RNA interference (RNAi), aptamer, CRISPR technology, polypeptide production, reprogramming, or antisense-based molecules for research or therapeutic purposes may be incorporated in various embodiments. Such modifications may, for example, increase stability (e.g., by reducing sensitivity to cleavage by nucleases), decrease clearance in vivo, increase cell uptake, or confer other properties that improve the translation, potency, efficacy, specificity, or otherwise render the nucleic acid more suitable for an intended use. Various non-limiting examples of nucleic acid modifications are described in, e.g., Deleavey G F, et al., Chemical modification of siRNA. Curr. Protoc. Nucleic Acid Chem. 2009; 39:16.3.1-16.3.22; Crooke, ST (ed.) Antisense drug technology: principles, strategies, and applications, Boca Raton: CRC Press, 2008; Kurreck, J. (ed.) Therapeutic oligonucleotides, RSC biomolecular sciences. Cambridge: Royal Society of Chemistry, 2008; U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929, 226; 5,977,296; 6,140,482; 6,455,308 and/or in PCT application publications WO 00/56746 and WO 01/14398. Different modifications may be used in the two strands of a double-stranded nucleic acid. A nucleic acid may be modified uniformly or on only a portion thereof and/or may contain multiple different modifications. Where the length of a nucleic acid or nucleic acid region is given in terms of a number of nucleotides (nt) it should be understood that the number refers to the number of nucleotides in a single-stranded nucleic acid or in each strand of a double-stranded nucleic acid unless otherwise indicated. An “oligonucleotide” is a relatively short nucleic acid, typically between about 5 and about 100 nt long. In some embodiments, the nucleic acid codes for MCART1, MCART2 or functional variants thereof.

In some aspects, the agent is a morpholino. Morpholinos are typically synthetic molecules, of about 25 bases in length and bind to complementary sequences of RNA by standard nucleic acid base-pairing. Morpholinos have standard nucleic acid bases, but those bases are bound to morpholine rings instead of deoxyribose rings and are linked through phosphorodiamidate groups instead of phosphates. Morpholinos do not degrade their target RNA molecules, unlike many antisense structural types (e.g., phosphorothioates, siRNA). Instead, morpholinos act by steric blocking and bind to a target sequence within a RNA and block molecules that might otherwise interact with the RNA. In some embodiments, the synthetic RNA is as described in WO 2017075406.

The term “RNA interference” (RNAi) encompasses processes in which a molecular complex known as an RNA-induced silencing complex (RISC) reduces gene expression in a sequence-specific manner in, e.g., eukaryotic cells, e.g., vertebrate cells, or in an appropriate in vitro system. RISC may incorporate a short nucleic acid strand (e.g., about 16- about 30 nucleotides (nt) in length) that pairs with and directs or “guides” sequence-specific degradation or translational repression of RNA (e.g., mRNA) to which the strand has complementarity. The short nucleic acid strand may be referred to as a “guide strand” or “antisense strand”. An RNA strand to which the guide strand has complementarity may be referred to as a “target RNA”. A guide strand may initially become associated with RISC components (in a complex sometimes termed the RISC loading complex) as part of a short double-stranded RNA (dsRNA), e.g., a short interfering RNA (siRNA). The other strand of the short dsRNA may be referred to as a “passenger strand” or “sense strand”. The complementarity of the structure formed by hybridization of a target RNA and the guide strand may be such that the strand can (i) guide cleavage of the target RNA in the RNA-induced silencing complex (RISC) and/or (ii) cause translational repression of the target RNA. Reduction of expression due to RNAi may be essentially complete (e.g., the amount of a gene product is reduced to background levels) or may be less than complete in various embodiments. For example, mRNA and/or protein level may be reduced by 50%, 60%, 70%, 75%, 80%, 85%, 90%, 95%, or more, in various embodiments. As known in the art, the complementarity between the guide strand and a target RNA need not be perfect (100%) but need only be sufficient to result in inhibition of gene expression. For example, in some embodiments 1, 2, 3, 4, 5, or more nucleotides of a guide strand may not be matched to a target RNA. “Not matched” or “unmatched” refers to a nucleotide that is mismatched (not complementary to the nucleotide located opposite it in a duplex, i.e., wherein Watson-Crick base pairing does not take place) or forms at least part of a bulge. Examples of mismatches include, without limitation, an A opposite a G or A, a C opposite an A or C, a U opposite a C or U, a G opposite a G. A bulge refers to a sequence of one or more nucleotides in a strand within a generally duplex region that are not located opposite to nucleotide(s) in the other strand. “Partly complementary” refers to less than perfect complementarity. In some embodiments a guide strand has at least about 80%, 85%, or 90%, e.g., least about 91%, 92%, 93%, 94%, 95%, 96%, 97%, 98%, 99%, or 100% sequence complementarity to a target RNA over a continuous stretch of at least about 15 nt, e.g., between 15 nt and 30 nt, between 17 nt and 29 nt, between 18 nt and 25 nt, between 19 nt and 23 nt, of the target RNA. In some embodiments at least the seed region of a guide strand (the nucleotides in positions 2-7 or 2-8 of the guide strand) is perfectly complementary to a target RNA. In some embodiments, a guide strand and a target RNA sequence may form a duplex that contains no more than 1, 2, 3, or 4 mismatched or bulging nucleotides over a continuous stretch of at least 10 nt, e.g., between 10-30 nt. In some embodiments a guide strand and a target RNA sequence may form a duplex that contains no more than 1, 2, 3, 4, 5, or 6 mismatched or bulging nucleotides over a continuous stretch of at least 12 nt, e.g., between 10-30 nt. In some embodiments, a guide strand and a target RNA sequence may form a duplex that contains no more than 1, 2, 3, 4, 5, 6, 7, or 8 mismatched or bulging nts over a continuous stretch of at least 15 nt, e.g., between 10-30 nt. In some embodiments, a guide strand and a target RNA sequence may form a duplex that contains no mismatched or bulging nucleotides over a continuous stretch of at least 10 nt, e.g., between 10-30 nt. In some embodiments, between 10-30 nt is 10, 11, 12, 13, 14, 15, 16, 17, 18, 19, 20, 21, 22, 23, 24, 25, 26, 27, 28, 29, or 30 nt.

As used herein, the term “RNAi agent” encompasses nucleic acids that can be used to achieve RNAi in eukaryotic cells. Short interfering RNA (siRNA), short hairpin RNA (shRNA), and microRNA (miRNA) are examples of RNAi agents. siRNAs typically comprise two separate nucleic acid strands that are hybridized to each other to form a structure that contains a double stranded (duplex) portion at least 15 nt in length, e.g., about 15- about 30 nt long, e.g., between 17-27 nt long, e.g., between 18-25 nt long , e.g., between 19-23 nt long, e.g., 15, 16, 17, 18, 19, 20, 21, 22, 23, 24, 25, 26, 27, 28, 29, or 30 nucleotides. In some embodiments the strands of an siRNA are perfectly complementary to each other within the duplex portion. In some embodiments the duplex portion may contain one or more unmatched nucleotides, e.g., one or more mismatched (non-complementary) nucleotide pairs or bulged nucleotides. In some embodiments either or both strands of an siRNA may contain up to about 1, 2, 3, or 4 unmatched nucleotides within the duplex portion. In some embodiments a strand may have a length of between 15-35 nt, e.g., between 17-29 nt, e.g., 19-25 nt, e.g., 21-23 nt. Strands may be equal in length or may have different lengths in various embodiments. In some embodiments strands may differ by between 1-10 nt in length. A strand may have a 5′ phosphate group and/or a 3′ hydroxyl (—OH) group. Either or both strands of an siRNA may comprise a 3′ overhang of, e.g., about 1-10 nt (e.g., 1-5 nt, e.g., 2 nt). Overhangs may be the same length or different in lengths in various embodiments. In some embodiments an overhang may comprise or consist of deoxyribonucleotides, ribonucleotides, or modified nucleotides or modified ribonucleotides such as 2′-O-methylated nucleotides, or 2′-O-methyl-uridine. An overhang may be perfectly complementary, partly complementary, or not complementary to a target RNA in a hybrid formed by the guide strand and the target RNA in various embodiments.

shRNAs are nucleic acid molecules that comprise a stem-loop structure and a length typically between about 40-150 nt, e.g., about 50-100 nt, e.g., 60-80 nt. A “stem-loop structure” (also referred to as a “hairpin” structure) refers to a nucleic acid having a secondary structure that includes a region of nucleotides which are known or predicted to form a double strand (stem portion; duplex) that is linked on one side by a region of (usually) predominantly single-stranded nucleotides (loop portion). Such structures are well known in the art and the term is used consistently with its meaning in the art. A guide strand sequence may be positioned in either arm of the stem, i.e., 5′ with respect to the loop or 3′ with respect to the loop in various embodiments. As is known in the art, the stem structure does not require exact base-pairing (perfect complementarity). Thus, the stem may include one or more unmatched residues or the base-pairing may be exact, i.e., it may not include any mismatches or bulges. In some embodiments the stem is between 15-30 nt, e.g., between 17-29 nt, e.g., 19-25 nt. In some embodiments the stem is between15-19 nt. In some embodiments the stem is between19-30 nt. The primary sequence and number of nucleotides within the loop may vary. Examples of loop sequences include, e.g., UGGU; ACUCGAGA; UUCAAGAGA. In some embodiments a loop sequence found in a naturally occurring miRNA precursor molecule (e.g., a pre-miRNA) may be used. In some embodiments a loop sequence may be absent (in which case the termini of the duplex portion may be directly linked). In some embodiments a loop sequence may be at least partly self-complementary. In some embodiments the loop is between 1 and 20 nt in length, e.g., 1-15 nt, e.g., 4-9 nt. The shRNA structure may comprise a 5′ or 3′ overhang. As known in the art, an shRNA may undergo intracellular processing, e.g., by the ribonuclease (RNase) III family enzyme known as Dicer, to remove the loop and generate an siRNA.

Mature endogenous miRNAs are short (typically 18-24 nt, e.g., about 22 nt), single-stranded RNAs that are generated by intracellular processing from larger, endogenously encoded precursor RNA molecules termed miRNA precursors (see, e.g., Bartel, D., Cell. 116(2):281-97 (2004); Bartel DP. Cell. 136(2):215-33 (2009); Winter, J., et al., Nature Cell Biology 11: 228 -234 (2009). Artificial miRNA may be designed to take advantage of the endogenous RNAi pathway in order to silence a target RNA of interest. The sequence of such artificial miRNA may be selected so that one or more bulges is present when the artificial miRNA is hybridized to its target sequence, mimicking the structure of naturally occurring miRNA:mRNA hybrids. Those of ordinary skill in the art are aware of how to design artificial miRNA.

An RNAi agent that contains a strand sufficiently complementary to an RNA of interest so as to result in reduced expression of the RNA of interest (e.g., as a result of degradation or repression of translation of the RNA) in a cell or in an in vitro system capable of mediating RNAi and/or that comprises a sequence that is at least 80%, 90%, 95%, or more (e.g., 100%) complementary to a sequence comprising at least 10, 12, 15, 17, or 19 consecutive nucleotides of an RNA of interest may be referred to as being “targeted to” the RNA of interest. An RNAi agent targeted to an RNA transcript may also considered to be targeted to a gene from which the transcript is transcribed.

In some embodiments an RNAi agent is a vector (e.g., an expression vector) suitable for causing intracellular expression of one or more transcripts that give rise to a siRNA, shRNA, or miRNA in the cell. Such a vector may be referred to as an “RNAi vector”. An RNAi vector may comprise a template that, when transcribed, yields transcripts that may form a siRNA (e.g., as two separate strands that hybridize to each other), shRNA, or miRNA precursor (e.g., pri-miRNA or pre-mRNA).

An RNAi agent may be produced in any of variety of ways in various embodiments. For example, nucleic acid strands may be chemically synthesized (e.g., using standard nucleic acid synthesis techniques) or may be produced in cells or using an in vitro transcription system. Strands may be allowed to hybridize (anneal) in an appropriate liquid composition (sometimes termed an “annealing buffer”). An RNAi vector may be produced using standard recombinant nucleic acid techniques.

In some embodiments, a catalytically inactive site specific nuclease and an effector domain capable of attaching a DNA, RNA, or protein to the nucleotide sequence is used as the agent. In some embodiments, the catalytically inactive site specific nuclease dCas (e.g., dCas9 or Cpf1) is used as the agent. The agent may reduce or increase expression of MCART1 (e.g., via modulating methylation of genomic DNA involved in expression of MCART1) or reduce or increase activity of MCART1 (e.g., by modifying the coding sequence for MCART1). The agent may reduce or increase expression of MCART2 (e.g., via modulating methylation of genomic DNA involved in expression of MCART2) or reduce or increase activity of MCART2 (e.g., by modifying the coding sequence for MCART2). In some embodiments, the agent modifies the nucleotide sequence in a cell (e.g., cancer cell) coding for lysine 91, arginine 182, and/or arginine 278 of MCART1. In some embodiments, the agent modifies the nucleotide sequence in a cell (e.g., cancer cell) coding for lysine 91 and converts the nucleotide sequence to code for alanine (K92A). In some embodiments, the agent is a dCas-transcription activator domain fusion protein that enhances transcription of MCART1 or MCART2 in the presence of the appropriate guide sequence.

A variety of CRISPR associated (Cas) genes or proteins which are known in the art can be modified to make a catalytically inactive site specific nuclease, the choice of Cas protein will depend upon the particular conditions of the method (e.g., ncbi.nlm.nih.gov/gene/?term=cas9). Specific examples of Cas proteins include Cas1, Cas2, Cas3, Cas4, Cas5, Cas6, Cas7, Cas8, Cas9 and Cas10. In a particular aspect, the Cas nucleic acid or protein used in the methods is Cas9. In some embodiments a Cas protein, e.g., a Cas9 protein, may be from any of a variety of prokaryotic species. In some embodiments a particular Cas protein, e.g., a particular Cas9 protein, may be selected to recognize a particular protospacer-adjacent motif (PAM) sequence. In certain embodiments a Cas protein, e.g., a Cas9 protein, may be obtained from a bacteria or archaea or synthesized using known methods. In certain embodiments, a Cas protein may be from a gram positive bacteria or a gram negative bacteria. In certain embodiments, a Cas protein may be from a Streptococcus, (e.g., a S. pyogenes, a S. thermophilus) a Crptococcus, a Corynebacterium, a Haemophilus, a Eubacterium, a Pasteurella, a Prevotella, a VeiUonella, or a Marinobacter. In some embodiments nucleic acids encoding two or more different Cas proteins, or two or more Cas proteins, may be introduced into a cell, zygote, embryo, or animal, e.g., to allow for recognition and modification of sites comprising the same, similar or different PAM motifs.

In some embodiments, the Cas protein is Cpf1 protein or a functional portion thereof. In some embodiments, the Cas protein is Cpf1 from any bacterial species or functional portion thereof. In certain embodiments, a Cpf1 protein is a Francisella novicida U112 protein or a functional portion thereof, a Acidaminococcus sp. BV3L6 protein or a functional portion thereof, or a Lachnospiraceae bacterium ND2006 protein or a function portion thereof. Cpf1 protein is a member of the type V CRISPR systems. Cpf1 protein is a polypeptide comprising about 1300 amino acids. Cpf1 contains a RuvC-like endonuclease domain

In some embodiments a Cas9 nickase may be generated by inactivating one or more of the Cas9 nuclease domains. In some embodiments, an amino acid substitution at residue 10 in the RuvC I domain of Cas9 converts the nuclease into a DNA nickase. For example, the aspartate at amino acid residue 10 can be substituted for alanine (Cong et al, Science, 339:819-823). Other amino acids mutations that create a catalytically inactive Cas9 protein includes mutating at residue 10 and/or residue 840. Mutations at both residue 10 and residue 840 can create a catalytically inactive Cas9 protein, sometimes referred herein as dCas9. For example, a D10A and a H840A Cas9 mutant is catalytically inactive.

As used herein an “effector domain” is a molecule (e.g., protein) that modulates the expression and/or activation of a genomic sequence (e.g., gene). The effector domain may have methylation activity or demethylation activity (e.g., DNA methylation or DNA demethylation activity). In some aspects, the effector domain targets one or both alleles of a gene. The effector domain can be introduced as a nucleic acid sequence and/or as a protein. In some aspects, the effector domain can be a constitutive or an inducible effector domain. In some aspects, a Cas (e.g., dCas) nucleic acid sequence or variant thereof and an effector domain nucleic acid sequence are introduced into a cell. In some aspects, the effector domain is fused to a molecule that associates with (e.g., binds to) Cas protein (e.g., the effector molecule is fused to an antibody or antigen binding fragment thereof that binds to Cas protein). In some aspects, a Cas (e.g., dCas) protein or variant thereof and an effector domain are fused or tethered creating a chimeric protein and are introduced into the cell as the chimeric protein. In some aspects, the Cas (e.g., dCas) protein and effector domain bind as a protein-protein interaction. In some aspects, the Cas (e.g., dCas) protein and effector domain are covalently linked. In some aspects, the effector domain associates non-covalently with the Cas (e.g., dCas) protein. In some aspects, a Cas (e.g., dCas) nucleic acid sequence and an effector domain nucleic acid sequence are introduced as separate sequences and/or proteins. In some aspects, the Cas (e.g., dCas) protein and effector domain are not fused or tethered.

In some embodiments, the catalytically inactive site specific nuclease can be guided to specific DNA sites by one or more RNA sequences (sgRNA) to modulate activity and/or expression of one or more genomic sequences (e.g., exert certain effects on transcription or chromatin organization, or bring specific kind of molecules into specific DNA loci, or act as sensor of local histone or DNA state). In specific aspects, fusions of a dCas9 tethered with all or a portion of an effector domain create chimeric proteins that can be guided to specific DNA sites by one or more RNA sequences to modulate or modify methylation or demethylation of one or more genomic sequences. As used herein, a “biologically active portion of an effector domain” is a portion that maintains the function (e.g. completely, partially, minimally) of an effector domain (e.g., a “minimal” or “core” domain). The fusion of the Cas9 (e.g., dCas9) with all or a portion of one or more effector domains created a chimeric protein.

Examples of effector domains include a transcription activation domain (e.g, Gal4, Oaf1, Leu3, Rtg3, Pho4, Gln3, Gcn4, p53, NFAT, NF-κB, or VP16 transcription activation domain), chromatin organizer domain, a remodeler domain, a histone modifier domain, a DNA modification domain, a RNA binding domain, a protein interaction input devices domain (Grunberg and Serrano, Nucleic Acids Research, 3 ′8 (8): ′2663 -267 ′5 (2010)), and a protein interaction output device domain (Grunberg and Serrano, Nucleic Acids Research, 3 ′8 (8): ′2663 -267 ′5 (2010)). In some aspects, the effector domain is a DNA modifier. Specific examples of DNA modifiers include 5hmc conversion from 5 mC such as Tetl (Tetl CD); DNA demethylation by Tetl, ACID A, MBD4, Apobecl, Apobec2, Apobec3, Tdg, Gadd45a, Gadd45b, ROS1; DNA methylation by Dnmtl, Dnmt3a, Dnmt3b, CpG Methyltransferase M.SssI, and/or M.EcoHK31I. In specific aspects, an effector domain is Tetl. In other specific aspects, as effector domain is Dmnt3a. In some embodiments, dCas9 is fused to Tetl. In other embodiments, dCas9 is fused to Dnmt3a. Other examples of effector domains are described in PCT Application No. PCT/US2014/034387 and U.S. application Ser. No. 14/785,031, which are incorporated herein by reference in their entirety. Methods of using catalytically inactive site specific nuclease, effector domains for modifying a nucleotide sequence (e.g., genomic sequence), and sgRNA are taught in PCT/US2017/065918 filed 12Dec. 2017, which is incorporated herein by reference.

In some embodiments, the agent is a small molecule. The term “small molecule” refers to an organic molecule that is less than about 2 kilodaltons (kDa) in mass. In some embodiments, the small molecule is less than about 1.5 kDa, or less than about 1 kDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups.

In some embodiments, the agent is a protein or polypeptide. The term “polypeptide” refers to a polymer of amino acids linked by peptide bonds. A protein is a molecule comprising one or more polypeptides. A peptide is a relatively short polypeptide, typically between about 2 and 100 amino acids (aa) in length, e.g., between 4 and 60 aa; between 8 and 40 aa; between 10 and 30 aa. The terms “protein”, “polypeptide”, and “peptide” may be used interchangeably. In general, a polypeptide may contain only standard amino acids or may comprise one or more non-standard amino acids (which may be naturally occurring or non-naturally occurring amino acids) and/or amino acid analogs in various embodiments. A “standard amino acid” is any of the 20 L-amino acids that are commonly utilized in the synthesis of proteins by mammals and are encoded by the genetic code. A “non-standard amino acid” is an amino acid that is not commonly utilized in the synthesis of proteins by mammals. Non-standard amino acids include naturally occurring amino acids (other than the 20 standard amino acids) and non-naturally occurring amino acids. An amino acid, e.g., one or more of the amino acids in a polypeptide, may be modified, for example, by addition, e.g., covalent linkage, of a moiety such as an alkyl group, an alkanoyl group, a carbohydrate group, a phosphate group, a lipid, a polysaccharide, a halogen, a linker for conjugation, a protecting group, a small molecule (such as a fluorophore), etc.

In some embodiments, the agent is a peptide mimetic. The terms “mimetic,” “peptide mimetic” and “peptidomimetic” are used interchangeably herein, and generally refer to a peptide, partial peptide or non-peptide molecule that mimics the tertiary binding structure or activity of a selected native peptide or protein functional domain (e.g., binding motif or active site). These peptide mimetics include recombinantly or chemically modified peptides, as well as non-peptide agents such as small molecule drug mimetics.

In some embodiments, the agent is encoded by a synthetic RNA (e.g., modified mRNAs). The synthetic RNA can encode any suitable agent described herein. Synthetic RNAs, including modified RNAs are taught in WO 2017075406, which is herein incorporated by reference. In some embodiments, the agent is a synthetic RNA.

In some embodiments, the agent increases or the decreases the expression of MCART1 or the activity of a gene product of MCART1 by at least about 5%, 10%, 15%, 20%, 25%, 30%, 35%, 40%, 45%, 50%, 55%, 60%, 65%, 70%, 75%, 80%, 85%, 90%, 95%, 99% or more. In some embodiments, the agent increases or the decreases the expression of MCART1 or the activity of a gene product of MCART1 by at least about 1.1 fold, at least 1.2 fold, 1.3 fold, at least 1.4 fold, at least 1.5 fold, at least 1.6 fold, at least 1.7 fold, at least 1.8 fold, at least 1.9 fold, at least 2 fold, at least 3 fold, at least 4 fold, at least 5 fold, at least 10 fold, at least 20 fold, at least 30 fold, at least 40 fold, at least 50 fold, or at least 100 fold, at least a 1,000 fold, at least 10,000 fold.

In some embodiments, the agent increases or the decreases the expression of MCART2 or the activity of a gene product of MCART2 by at least about 5%, 10%, 15%, 20%, 25%, 30%, 35%, 40%, 45%, 50%, 55%, 60%, 65%, 70%, 75%, 80%, 85%, 90%, 95%, 99% or more. In some embodiments, the agent increases or the decreases the expression of MCART2 or the activity of a gene product of MCART2 by at least about 1.1 fold, at least 1.2 fold, 1.3 fold, at least 1.4 fold, at least 1.5 fold, at least 1.6 fold, at least 1.7 fold, at least 1.8 fold, at least 1.9 fold, at least 2 fold, at least 3 fold, at least 4 fold, at least 5 fold, at least 10 fold, at least 20 fold, at least 30 fold, at least 40 fold, at least 50 fold, or at least 100 fold, at least a 1,000 fold, at least 10,000 fold.

In some embodiments, the agent comprises, consists essentially of, or consists of a peptide, nucleic acid, small molecule, or hybrid thereof.

An “effective amount” or “effective dose” of an agent (or composition containing such agent) refers to the amount sufficient to achieve a desired biological and/or pharmacological effect, e.g., when delivered to a cell or organism according to a selected administration form, route, and/or schedule. As will be appreciated by those of ordinary skill in this art, the absolute amount of a particular agent or composition that is effective may vary depending on such factors as the desired biological or pharmacological endpoint, the agent to be delivered, the target tissue, etc. Those of ordinary skill in the art will further understand that an “effective amount” may be contacted with cells or administered in a single dose, or through use of multiple doses, in various embodiments. A biological effect may be, e.g., reducing expression or activity of one or more gene products, reducing activity of a metabolic pathway or reaction, reducing cell proliferation or survival of cells (e.g., tumor cell proliferation or survival), reducing tumor maintenance, size, growth, or progression.

“Contacting”, “contacting a cell” and similar terms as used herein, refer to any means of introducing an agent (e.g., a nucleic acid, peptide, antibody, small molecule, etc.) into a target cell, including chemical and physical means, whether directly or indirectly or whether the agent physically contacts the cell directly or is introduced into an environment in which the cell is present. Contacting is intended to encompass methods of exposing a cell, delivering to a cell, or “loading” a cell with an agent by viral (e.g., AAV or Lentivirus) or non-viral vectors, wherein such agent is bioactive upon delivery or wherein such agent is processed intracellularly to an active form. The method of delivery will be chosen for the particular agent and use (e.g., cancer being treated). Parameters that affect delivery, as is known in the medical art, can include, inter alia, the cell type affected, and cellular location. In some embodiments, contacting includes administering the agent to a subject.

Some aspects of the disclosure are related to a method of treating or preventing a disease, disorder or dysfunction associated with an aberrant level of NAD in mitochondria of a subject, comprising administering to the subject an agent that modulates the expression of MCART1 or the activity of a gene product of MCART1. Some aspects of the disclosure are related to a method of treating or preventing a disease, disorder or dysfunction associated with an aberrant level of NAD in mitochondria of a subject, comprising administering to the subject an agent that modulates the expression of MCART2 or the activity of a gene product of MCART2. The agent is not limited and may be any agent described herein. In some embodiments, the level of NAD is stabilized (e.g., the rate of increase or decrease of an NAD level in a mitochondria is reduced or stopped, fluctuations in NAD levels are reduced or eliminated). In some embodiments, the expression of MCART1 or the activity of a gene product of MCART1 is increased, thereby increasing the level of NAD in the mitochondria. In some embodiments, the expression of MCART1 or the activity of a gene product of MCART1 is decreased, thereby decreasing the level of NAD in the mitochondria. In some embodiments, the expression or activity of MCART2 is increased, thereby increasing the level of NAD in mitochondria. An aberrant level of NAD is a level of NAD that is less than or more than the level of NAD in a mitochondria of a healthy cell or a non-cancerous cell. An aberrant level of NAD may be due to a defect in mitochondrial MCART1 expression or activity which increases or decreases a level of NAD as compared to the level of NAD in mitochondria without the defect.

As used herein, a “subject” is a mammal, including but not limited to a primate (e.g., a human), rodent (e.g., mouse or rat) dog, cat, horse, cow, pig, sheep, goat, or chicken. Preferred subjects are human subjects. The human subject may be a pediatric or adult subject. In some embodiments, the subject is elderly. In some embodiments, the subject is at least about 40 years old, at least about 45 years old, at least about 50 years old, at least about 55 years old, at least about 60 years old, at least about 65 years old, at least about 70 years old, at least about 75 years old, at least about 80 years old, at least about 85 years old, or at least about 90 years old. In some embodiments, the subject has been diagnosed with, is suspected of having, or is at risk of having a disease or disorder described herein. In some embodiments, the subject has cancer.

In some embodiments, the disease or disorder is a mitochondrial disease or disorder, a metabolic disease or disorder, a cardiovascular disease or disorder, a muscular disease or disorder, a neurological disease or disorder, a disease or disorder associated with fatigue, or a disease or disorder associated with aging.

The type of mitochondrial disease is not limited. In some embodiments the mitochondrial disease exhibits changes in the one-carbon metabolism pathway. Non-limiting examples of mitochondrial diseases include mitochondrial myopathy; diabetes (e.g., diabetes mellitus and deafness (DAD)); Leber's hereditary optic neuropathy (LHON); Leigh syndrome, subacute sclerosing encephalopathy; neuropathy, ataxia, retinis, pigmentosa, and ptosis (NARP); myoneurogenic gastrointestinal encephalopathy (MNGIE); myoclonic epilepsy with ragged red fibers (MERRF); mtDNA depletion (e.g., mitochondrial neurogastrointestinal encephalomyopathy (MNGIE)); Huntington's disease; cancer; Alzheimer's disease; Parkinson's disease; bipolar disorder; schizophrenia; agent and senescence; anxiety disorders; cardiovascular disease; sarcopenia; and chronic fatigue syndrome. In some embodiments, the mitochondrial disease is a complex I deficiency.

Metabolic disorder, as used herein, shall mean any disease or disorder that damages or interferes with normal function in a cell, tissue, or organ by affecting the production of energy in cells or the accumulation of toxins in a cell, tissue, organ, or individual. Metabolic disorders include, but are not limited to, type II diabetes, metabolic syndrome, hyperglycemia, and obesity.

Cardiovascular diseases and disorders are also not limited. Non-limiting examples of cardiovascular conditions include diastolic heart failure, cardiac hypertrophy, hypertension, valvular disease, aortic stenosis, and genetic hypertrophic cardiomyopathy.

Muscle diseases and disorders are not limited. Examples of muscle diseases and disorders which may be treated include skeletal muscle diseases and disorders such as myopathies, dystrophies, myoneural conductive diseases, traumatic muscle injury, and nerve injury. Cardiac muscle pathologies such as cardiomyopathies, ischemic damage, congenital disease, and traumatic injury may also be treated using the methods of the invention, as may smooth muscle diseases and disorders such as arterial sclerosis, vascular lesions, and congenital vascular diseases.

The neurological disease, condition, or disorder is not limited. As used herein, a neurological disease, condition, or disorder refers to a disease condition involving neuronal loss mediated or characterized at least partially by at least one of deterioration of neural stem cells and/or progenitor cells, or a decreased capacity for neurogenesis. Non-limiting examples include polyglutamine expansion disorders (e.g., HD, dentatorubropallidoluysian atrophy, Kennedy's disease (also referred to as spinobulbar muscular atrophy), and spinocerebellar ataxia (e.g., type 1, type 2, type 3 (also referred to as Machado-Joseph disease), type 6, type 7, and type 17)), other trinucleotide repeat expansion disorders (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, Alzheimer disease, amyotrophic lateral sclerosis (ALS), ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, Guillain-Barré syndrome, ischemia stroke, Krabbe disease, kuru, Lewy body dementia, multiple sclerosis, multiple system atrophy, non-Huntingtonian type of Chorea, Parkinson's disease, Pelizaeus-Merzbacher disease, Pick's disease, primary lateral sclerosis, progressive supranuclear palsy, Refsum's disease, Sandhoff disease, Schilder's disease, spinal cord injury, spinal muscular atrophy (SMA), SteeleRichardson-Olszewski disease, and Tabes dorsalis. In certain contexts, neurodegenerative disorders encompass neurological injuries or damages to the CNS or the PNS associated with physical injury (e.g., head trauma, mild to severe traumatic brain injury (TBI), spinal cord injury, diffuse axonal injury, craniocerebral trauma, cranial nerve injuries, cerebral contusion, intracerebral haemorrhage and acute brain swelling), ischemia (e.g., resulting from spinal cord infarction or ischemia, ischemic infarction, stroke, cardiac insufficiency or arrest, atherosclerotic thrombosis, ruptured aneurysm, embolism or haemorrhage), certain medical procedures or exposure to biological or chemic toxins or poisons (e.g., surgery, coronary artery bypass graft (CABG), electroconvulsive therapy, radiation therapy, chemotherapy, anti-neoplastic drugs, immunosuppressive agents, psychoactive, sedative or hypnotic drugs, alcohol, bacterial or industrial toxins, plant poisons, and venomous bites and stings), tumors (e.g., CNS metastasis, intraaxial tumors, primary CNS lymphomas, germ cell tumors, infiltrating and localized gliomas, fibrillary astrocytomas, oligodendrogliomas, ependymomas, pleomorphic xanthoastrocytomas, pilocytic astrocytomas, extraaxial brain tumors, meningiomas, schwannomas, neurofibromas, pituitary tumors, and mesenchymal tumors of the skull, spine and dura matter), infections (e.g., bacterial, viral, fungal, parasitic or other origin is selected from the group consisting of pyrogenic infections, meningitis, tuberculosis, syphilis, encephalomyelitis and leptomeningitis), metabolic or nutritional disorders (e.g., glycogen storage diseases, acid lipase diseases, Wemicke's or Marchiafava-Bignami's disease, Lesch-Nyhan syndrome, Farber's disease, gangliosidoses, vitamin B12 and folic acid deficiency), cognition or mood disorders (e.g., learning or memory disorder, bipolar disorders and depression), and various medical conditions associated with neural damage or destruction (e.g., asphyxia, prematurity in infants, perinatal distress, gaseous intoxication for instance from carbon monoxide or ammonia, coma, hypoglycaemia, dementia, epilepsy and hypertensive crises).

Diseases and disorders associated with fatigue are not limited. In some embodiments, the disease associated with fatigue includes Myalgic encephalomyelitis/chronic fatigue syndrome (ME/CFS).

Diseases and disorders associated with aging are not limited and refers to any disease, disorder, or undesirable state whose incidence in a population or severity in an individual correlates with the progression of age. In some embodiments, the age-related condition is a cardiovascular condition, aging of the heart, aging of skeletal muscle, or aging of the brain. Aging of any given organ can include, but is not limited to, reduced cellularity, reduced stem cell genomic integrity, reduced cellular function (e.g., reduced muscle contraction in muscle tissue), reduced regenerative capacity, atrophy (e.g., aging of the skin can include atrophy of the epidermis and/or sebaceous follicles). An age-related condition can be one that reduces the function of a given organ or one that is aesthetically undesirable (e.g., aging of the skin or muscle can be aesthetically undesirable). Additional age-related conditions can include, but are not limited to, sarcopenia, skin atrophy, muscle wasting, brain atrophy, atherosclerosis, arteriosclerosis, pulmonary emphysema, osteoporosis, osteoarthritis, immunologic incompetence, high blood pressure, dementia, Huntington's disease, Alzheimer's disease, cataracts, age-related macular degeneration, prostate cancer, stroke, diminished life expectancy, memory loss, wrinkles, impaired kidney function, and age-related hearing loss.

As used herein “treatment” or “treating,” in reference to a subject, includes amelioration, cure, and/or maintenance of a cure (i.e., the prevention or delay of relapse and/or reducing the likelihood of recurrence) of a disorder. Treatment after a disorder has started aims to reduce, ameliorate or altogether eliminate the disorder, and/or its associated symptoms, to prevent it from becoming worse, to slow the rate of progression, or to prevent the disorder from re-occurring once it has been initially eliminated (i.e., to prevent a relapse). Treating encompasses administration of an agent that may not have an effect on the disorder by itself but increases the efficacy of a second agent administered to the subject. A suitable dose and therapeutic regimen may vary depending upon the specific agent used, the mode of delivery of the compound, and whether it is used alone or in combination.

The dosage, administration schedule and method of administering the agent are not limited. In certain embodiments a reduced dose may be used when two or more agents are administered in combination either concomitantly or sequentially. The absolute amount will depend upon a variety of factors including other treatment(s), the number of doses and the individual patient parameters including age, physical condition, size and weight. These are factors well known to those of ordinary skill in the art and can be addressed with no more than routine experimentation. In some embodiments, a maximum tolerated dose may be used, that is, the highest safe and tolerable dose according to sound medical judgment. Therapeutic doses of anticancer agents are well known in the field of medicine for the treatment of cancer.

As used herein, pharmaceutical compositions comprise one or more agents or compositions that have therapeutic utility, and a pharmaceutically acceptable carrier, e.g., a carrier that facilitates delivery of agents or compositions. Agents and pharmaceutical compositions disclosed herein may be administered by any suitable means such as orally, intranasally, subcutaneously, intramuscularly, intravenously, intra-arterially, parenterally, intraperitoneally, intrathecally, intratracheally, ocularly, sublingually, vaginally, rectally, dermally, or as an aerosol. Depending upon the type of condition to be treated, compounds of the invention may, for example, be inhaled, ingested or administered by systemic routes. Thus, a variety of administration modes, or routes, are available. The particular mode selected will typically depend on factors such as the particular compound selected, the particular condition being treated and the dosage required for therapeutic efficacy. The methods described herein, generally speaking, may be practiced using any mode of administration that is medically acceptable, meaning any mode that produces acceptable levels of efficacy without causing clinically unacceptable adverse effects. Preferred modes of administration are parenteral and oral routes. The term “parenteral” includes subcutaneous, intravenous, intramuscular, intraperitoneal, and intrasternal injection, or infusion techniques. In some embodiments, inhaled medications are of particular use because of the direct delivery to the lung, for example in lung cancer patients. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers. In some embodiments, agents are delivered by pulmonary aerosol. Other appropriate routes will be apparent to one of ordinary skill in the art.

Some embodiments comprise administering to a subject a therapeutically effective amount of an agent described herein and a second therapeutic agent. “Administered in combination” means that two or more agents are administered to a subject. Such administration is sometimes referred to herein as “combination therapy”, “combined administration”, or “coadministration”. The agents may be administered in the same composition or separately. When they are co-administered, agents may be administered simultaneously or sequentially and in either instance, may be administered separately or in the same composition, e.g., a unit dosage form that includes both a MCART1 inhibitor and an anti-cancer agent. When administered separately, the agents may be administered in any order, provided that they are given sufficiently close in time to have a desired effect. “Therapeutically effective amounts” of agents administered in combination means that the amounts administered are therapeutically effective at least when the agents are administered in combination or as part of a treatment regimen that includes the agents and one or more additional agents. In some embodiments, administration in combination of first and second agents is performed such that (i) a dose of the second agent is administered before more than 90% of the most recently administered dose of the first agent has been metabolized to an inactive form or excreted from the body; or (ii) doses of the first and second agent are administered within 48 hours of each other, or (iii) the agents are administered during overlapping time periods (e.g., by continuous or intermittent infusion); or (iv) any combination of the foregoing. In some embodiments, three or more agents are administered and the afore-mentioned criteria are met with respect to all agents, or in some embodiments, the criteria are met if each agent is considered a “second agent” with respect to at least one other agent of the combination. In some embodiments, agents may be administered individually at substantially the same time (e.g., within less than 1, 2, 5, or 10 minutes of one another). In some embodiments they may be administered individually within a short time of one another (by which is meant less than 3 hours, sometimes less than 1 hour, sometimes within 10 or 30 minutes apart). In some embodiments, agents may be administered one or more times within 1, 2, 3, 4, 5, or 6 weeks of each other. In certain embodiments of combination therapy, the first agent is administered during the entire course of administration of the second agent; where the first agent is administered for a period of time that is overlapping with the administration of the second agent, e.g. where administration of the first agent begins before the administration of the second agent and the administration of the first agent ends before the administration of the second agent ends; where the administration of the second agent begins before the administration of the first agent and the administration of the second agent ends before the administration of the first agent ends; where the administration of the first agent begins before administration of the second agent begins and the administration of the second agent ends before the administration of the first agent ends; where the administration of the second agent begins before administration of the first agent begins and the administration of the first agent ends before the administration of the second agent ends. In some embodiments, agents may be administered in alternate weeks. The agents may, but need not, be administered by the same route of administration. A treatment course might include one or more treatment cycles, each of which may include one or more doses of a first agent, and one or more doses of a second agent.

Some aspects of the disclosure are related to a method of treating or preventing a disease or disorder associated with aging in a subject in need thereof, comprising administering to the subject an agent that modulates the expression of MCART1 or the activity of a gene product of MCART1 in the subject. The agent is not limited and may be any agent described herein. In some embodiments, the agent increases the expression of MCART2 or the activity of a gene product of MCART2. In some embodiments, the disease or disorder associated with aging is a cardiovascular disease or disorder, a neurological disease or disorder, a metabolic disease or disorder, or a muscular disease or disorder. The diseases and disorders are not limited and may be any disease or disorder described herein. In some embodiments, administration of the agent stabilizes or increases the level of NAD in mitochondria of the subject.

In some embodiments, the method further comprises administration of a second agent that increases cytoplasmic NAD levels or levels of a NAD precursor when administered to the subject. In some embodiments, the second agent is NADH, an intermediate of a de novo pathway for synthesizing NAD, an intermediate of a NAD salvage pathway, an intermediate of a nicotinamide riboside kinase pathway or combinations thereof. In some embodiments, the second agent is NAD or a precursor thereof. In some embodiments, the agent is nicotinamide mononucleotide, nicotinic acid mononucleotide or nicotinamide riboside. In some embodiments, the second agent is nicotinamide riboside.

Some aspects of the disclosure are related to a method of retaining or increasing exercise capacity or reducing fatigue in a subject in need thereof, comprising administering to the subject an agent that modulates the expression of MCART1 or the activity of a gene product of MCART1. The agent is not limited and may be any agent described herein. In some embodiments, the agent increases the expression of MCART2 or the activity of a gene product of MCART2. In some embodiments, administration of the agent stabilizes or increases the level of NAD in mitochondria of the subject. In some embodiments, the subject has reduced exercise capacity or increased fatigue due to aging. In some embodiments, the method further comprises administration of a second agent that increases cytoplasmic NAD levels or levels of a NAD precursor when administered to the subject. The second agent is not limited and may be any second agent described herein.

Some aspects of the disclosure are related to a method of inhibiting the growth or viability of a cancer cell, comprising contacting the cancer cell with an agent that reduces the expression of MCART1 or the activity of a gene product of MCART1. The agent is not limited and may be any agent described herein. In some embodiments, the agent inhibits the expression of MCART2 or the activity of a gene product of MCART2. In some embodiments, the agent modifies the nucleotide sequence in a cell (e.g., cancer cell) coding for lysine 91, arginine 182, and/or arginine 278 of MCART1. In some embodiments, the agent modifies the nucleotide sequence in a cell (e.g., cancer cell) coding for lysine 91 and converts the nucleotide sequence to code for alanine (K92A). In some embodiments, the agent is a mutant MCART1 or a fragment or derivative thereof without NAD transport activity, or a nucleic acid coding the same. In some embodiments, the mutant MCART1 has a substitution at a lysine at position 91. In some embodiments, the substitution at position 91 is for alanine (K91A). In some embodiments, the method reduces cancer cell growth or viability by at least about 5%, 10%, 15%, 20%, 25%, 30%, 35%, 40%, 45%, 50%, 55%, 60%, 65%, 70%, 75%, 80%, 85%, 90%, 95%, 99% or more. In some embodiments, the method completely inhibits growth of the cancer cell. In some embodiments, the method eradicates the cancer. In some embodiments, the method inhibits the growth or viability of cancer stem cells.

In some embodiments, the cancer cell (e.g., a tumor cell) is a brain tumor cell, e.g., a glioblastoma cell. In some embodiments, a tumor cell is a bladder tumor cell, breast tumor cell, cervical tumor cell, colorectal tumor cell, embryonal tumor cell, gastric tumor cell, germ cell tumor cell, head and neck tumor cell, hematologic tumor cell, kidney tumor cell, melanoma cell, mesothelial tumor cell, ovarian tumor cell, yolk sac tumor cell, or sarcoma cell. In some embodiments a breast tumor cell is a triple negative breast tumor cell. As known in the art, a “triple negative” breast tumor is a breast tumor that does not express estrogen receptor (ER), progesterone receptor (PR), or Her2/neu. In general, triple negative breast tumors typically have a worse prognosis than breast tumor that are not triple negative. In some embodiments, the cancer cell is a cancer stem cell. In some embodiments, the cancer cell is an adipose, brain, blood, epithelial, colon, heart, kidney, liver, lung, muscle, nerve, ovary, pancreas, small intestine, spleen, stomach, testis, or uterus cancer cell.

In some embodiments, the agent is contacted with the cancer cell in vivo (i.e., the agent is administered to a subject having cancer). The type of cancer is not limited. In some embodiments, cancer includes, but is not limited to: breast cancer; biliary tract cancer; bladder cancer; brain cancer (e.g., glioblastomas, medulloblastomas); cervical cancer; choriocarcinoma; colon cancer; endometrial cancer; esophageal cancer; gastric cancer; hematological neoplasms including acute lymphocytic leukemia and acute myelogenous leukemia; T-cell acute lymphoblastic leukemia/lymphoma; hairy cell leukemia; chronic lymphocytic leukemia, chronic myelogenous leukemia, multiple myeloma; adult T-cell leukemia/lymphoma; intraepithelial neoplasms including Bowen's disease and Paget's disease; liver cancer; lung cancer; lymphomas including Hodgkin's disease and lymphocytic lymphomas; neuroblastoma; melanoma, oral cancer including squamous cell carcinoma; ovarian cancer including ovarian cancer arising from epithelial cells, stromal cells, germ cells and mesenchymal cells; neuroblastoma, pancreatic cancer; prostate cancer; rectal cancer; sarcomas including angiosarcoma, gastrointestinal stromal tumors, leiomyosarcoma, rhabdomyosarcoma, liposarcoma, fibrosarcoma, and osteosarcoma; renal cancer including renal cell carcinoma and Wilms tumor; skin cancer including basal cell carcinoma and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullary carcinoma.

In some embodiments, the cancer cell or cancer relies upon, or partially relies upon, oxidative phosphorylation (OXPHOS) for ATP production. In some embodiments, the cancer cell or cancer is sensitive to an OXPHOS inhibitor. In some embodiments, the OXPHOS inhibitor is atovaquone, methylmalonate, 3-nitropropionic acid, rotenone, or metformin. In some embodiments, the cancer cell or cancer relies upon, or partially relies upon, glycolysis for ATP production (i.e., a glycolytic cancer). In some embodiments, the glycolytic cancer is also contacted with an inhibitor of glycolysis. In some embodiments, the inhibitor of glycolysis is 2-Deoxy-D-Glucose, 3-Bromopyruvic acid, 6-Aminonicotinamide, Lonidamine, Oxythiamine Chloride Hydrochloride, or Shikonin.

In some embodiments, the method further comprises contacting the cancer cell with a second agent having anti-cancer activity. The anti-cancer agent is not limited. In some embodiments, the anti-cancer agent is selected from: chemotherapy agents, antibody-based agents, kinase inhibitors (e.g., tyrosine kinase inhibitors, serine/threonine kinase inhibitors, etc.), immunomodulatory agents, biologic agents, and combinations thereof. A single additional agent or multiple additional agents or treatment modalities may be co-administered (at the same or differing time points and/or via the same or differing routes of administration and/or on the same or a differing dosing schedule).

In some embodiments, the chemotherapy agent is selected from but not limited to: actinomycin D, aldesleukin, alitretinoin, all-trans retinoic acid/ATRA, altretamine, amascrine, asparaginase, azacitidine, azathioprine, bacillus calmette-guerin/BCG, bendamustine hydrochloride, bexarotene, bicalutamide, bleomycin, bortezomib, busulfan, capecitabine, carboplatin, carfilzomib, carmustine, chlorambucil, cisplatin/cisplatinum, cladribine, cyclophosphamide/cytophosphane, cytabarine, dacarbazine, daunorubicin/daunomycin, denileukin diftitox, dexrazoxane, docetaxel, doxorubicin, epirubicin, etoposide, fludarabine, fluorouracil (5-FU), gemcitabine, goserelin, hydrocortisone, hydroxyurea, idarubicin, ifosfamide, interferon alfa, irinotecan CPT-11, lapatinib, lenalidomide, leuprolide, mechlorethamine/chlormethine/mustine/HN2, mercaptopurine, methotrexate, methylprednisolone, mitomycin, mitotane, mitoxantrone, octreotide, oprelvekin, oxaliplatin, paclitaxel, pamidronate, pegaspargase, pegfilgrastim, PEG interferon, pemetrexed, pentostatin, phenylalanine mustard, plicamycin/mithramycin, prednisone, prednisolone, procarbazine, raloxifene, romiplostim, sargramostim, streptozocin, tamoxifen, temozolomide, temsirolimus, teniposide, thalidomide, thioguanine, thiophosphoamide/thiotepa, thiotepa, topotecan hydrochloride, toremifene, tretinoin, valrubicin, vinblastine, vincristine, vindesine, vinorelbine, vorinostat, zoledronic acid, and combinations thereof. In some embodiments, the antibody-based agent is selected from but not limited to: alemtuzumab, bevacizumab, cetuximab, fresolimumab, gemtuzumab ozogamicin, ibritumomab tiuxetan, ipilimumab, ofatumumab, panitumumab, rituximab, tositumomab, trastuzumab, trastuzumab DM1, and combinations thereof. In some embodiments, the kinase inhibitor (e.g., tyrosine kinase inhibitors, serine/threonine kinase inhibitors, etc.) is selected from but not limited to: axitinib, bafetinib, bosutinib, cediranib, crizotinib, dasatinib, erlotinib, gefitinib, imatinib, lapatinib, neratinib, nilotinib, pazopanib, ponatinib, quizartinib, regorafenib, sorafenib, sunitinib, vandetanib, vatalanib, vemurafinib, and combinations thereof. In some embodiments, the kinase inhibitor is a Janus kinase inhibitor selected from but not limited to: AC-430, AZD1480, baricitinib, BMS-911453, CEP-33779, CYT387, GLPG-0634, INCB18424, lestaurtinib, LY2784544, NS-018, pacritinib, ruxolitinib, TG101348 (SAR302503), tofacitinib, VX-509, R-348, R723 and combinations thereof. In some embodiments, the immunomodulatory agent is selected from but not limited to: thalidomide, lenalidomide, pomalidomide, methotrexate, leflunomide, cyclophosphamide, cyclosporine A, minocycline, azathioprine, tacrolimus, methylprednisolone, mycophenolate mofetil, rapamycin, mizoribine, deoxyspergualin, brequinar, 5,6-dimethylxanthenone-4-acetic acid (DMXAA), lactoferrin, poly AU, polyI:polyC12U, poly-ICLC, imiquimod, resiquimod, unmethylated CpG dinucleotide (CpG-ODN), and ipilumumab. In some embodiments, the anti-cancer agent is selected from but not limited to: IL-2, IL-3, erythropoietin, G-CSF, filgrastim, interferon alfa, bortezomib and combinations thereof. In some embodiments, the anti-cancer agent is selected from but not limited to: AB0024, AZD1480, AT-9283, BMS-911543, CYT387, everolimus, givinostat, imetelstat, lestaurtinib, LY2784544, NS-018, oral arsenic, pacritinib, panobinostat, peginterferon alfa-2a, pomalidomide, pracinostat, ruxolitinib, TAK-901, and TG101438 (SAR302503).

In some embodiments, the anti-cancer agent inhibits the expression or activity of complex I. In some embodiments, the anti-cancer agent is an amiloride, an amiloride derivative (e.g., EIPA, MIA, benzamil), or a biguanide derivative (e.g., metformin, guanidine galegine, synthalin A, phenformin, proguanil, cycloguanil). See, Murai et al., Biochimica et Biophysica Acta (BBA)—Bioenergetics, Vol. 1857, No. 7 (2016) pp. 884-891, herein incorporated by reference in its entirety.

Compositions

Some aspects of the disclosure are related to a composition comprising an agent that that modulates the expression of MCART1 or the activity of a gene product of MCART1 when administered to a subject. The agent may be any agent, or combination of agents, disclosed herein. In some embodiments, the agent stabilizes the level of NAD (e.g., the rate of increase or decrease of an NAD level in a mitochondria is reduced or stopped, fluctuations in NAD levels are reduced or eliminated). In some embodiments, the agent increases expression of MCART1 or the activity of a gene product of MCART1, thereby increasing the level of NAD in the mitochondria. In some embodiments, the agent reduces the expression of MCART1 or the activity of a gene product of MCART1, thereby decreasing the level of NAD in the mitochondria. In some embodiments, the agent increases expression of MCART2 or the activity of a gene product of MCART2, thereby increasing the level of NAD in the mitochondria.

In some embodiments, the composition further comprises a second agent. The second agent may be a therapeutic agent (e.g., anti-cancer agent). In some embodiments, the second agent increases cytoplasmic NAD or NAD precursor levels. The second agent is not limited and may be any agent described herein. In some embodiments, the second agent is nicotinamide riboside. In some embodiments, the second agent is an anti-cancer agent.

In addition to the active agent(s), the pharmaceutical compositions typically comprise a pharmaceutically-acceptable carrier. The term “pharmaceutically-acceptable carrier”, as used herein, means one or more compatible solid or liquid vehicles, fillers, diluents, or encapsulating substances which are suitable for administration to a human or non-human animal. In preferred embodiments, a pharmaceutically-acceptable carrier is a non-toxic material that does not interfere with the effectiveness of the biological activity of the active ingredients. The term “compatible”, as used herein, means that the components of the pharmaceutical compositions are capable of being comingled with an agent, and with each other, in a manner such that there is no interaction which would substantially reduce the pharmaceutical efficacy of the pharmaceutical composition under ordinary use situations. Pharmaceutically-acceptable carriers should be of sufficiently high purity and sufficiently low toxicity to render them suitable for administration to the human or non-human animal being treated.

Some examples of substances which can serve as pharmaceutically-acceptable carriers are pyrogen-free water; isotonic saline; phosphate buffer solutions; sugars such as lactose, glucose, and sucrose; starches such as corn starch and potato starch; cellulose and its derivatives, such as sodium carboxymethylcellulose, ethylcellulose, cellulose acetate; powdered tragacanth; malt; gelatin; talc; stearic acid; magnesium stearate; calcium sulfate; vegetable oils such as peanut oil, cottonseed oil, sesame oil, olive oil, corn oil and oil of theobrama; polyols such as propylene glycol, glycerin, sorbitol, mannitol, and polyethylene glycol; sugar; alginic acid; cocoa butter (suppository base); emulsifiers, such as the Tweens; as well as other non-toxic compatible substances used in pharmaceutical formulation. Wetting agents and lubricants such as sodium lauryl sulfate, as well as coloring agents, flavoring agents, excipients, tableting agents, stabilizers, antioxidants, and preservatives, can also be present. It will be appreciated that a pharmaceutical composition can contain multiple different pharmaceutically acceptable carriers.

A pharmaceutically-acceptable carrier employed in conjunction with the compounds described herein is used at a concentration or amount sufficient to provide a practical size to dosage relationship. The pharmaceutically-acceptable carriers, in total, may, for example, comprise from about 60% to about 99.99999% by weight of the pharmaceutical compositions, e.g., from about 80% to about 99.99%, e.g., from about 90% to about 99.95%, from about 95% to about 99.9%, or from about 98% to about 99%.

Pharmaceutically-acceptable carriers suitable for the preparation of unit dosage forms for oral administration and topical application are well-known in the art. Their selection will depend on secondary considerations like taste, cost, and/or shelf stability, which are not critical for the purposes of the subject invention, and can be made without difficulty by a person skilled in the art.

Pharmaceutically acceptable compositions can include diluents, fillers, salts, buffers, stabilizers, solubilizers and other materials which are well-known in the art. The choice of pharmaceutically-acceptable carrier to be used in conjunction with the compounds of the present invention is basically determined by the way the compound is to be administered. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof in certain embodiments. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts. It will also be understood that a compound can be provided as a pharmaceutically acceptable pro-drug, or an active metabolite can be used. Furthermore, it will be appreciated that agents may be modified, e.g., with targeting moieties, moieties that increase their uptake, biological half-life (e.g., pegylation), etc.

The agents may be administered in pharmaceutically acceptable solutions, which may routinely contain pharmaceutically acceptable concentrations of salt, buffering agents, preservatives, compatible carriers, adjuvants, and optionally other therapeutic ingredients.

The agents may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

Compositions suitable for oral administration may be presented as discrete units, such as capsules, tablets, lozenges, each containing a predetermined amount of the active agent. Other compositions include suspensions in aqueous liquids or non-aqueous liquids such as a syrup, elixir or an emulsion.

In some embodiments, agents may be administered directly to a tissue, e.g., a tissue in which the cancer cells are found or one in which a cancer is likely to arise. Direct tissue administration may be achieved by direct injection. The agents may be administered once, or alternatively they may be administered in a plurality of administrations. If administered multiple times, the agents may be administered via different routes. For example, the first (or the first few) administrations may be made directly into the affected tissue while later administrations may be systemic.

For oral administration, compositions can be formulated readily by combining the active agent(s) with pharmaceutically acceptable carriers well known in the art. Such carriers enable the agents to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration. For buccal administration, the compositions may take the form of tablets or lozenges formulated in conventional manner.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

In certain embodiments, the vehicle is a biocompatible microparticle or implant that is suitable for implantation into the mammalian recipient. Exemplary bioerodible implants that are useful in accordance with this method are described in PCT International Application Publication No. WO 95/24929, entitled “Polymeric Gene Delivery System”, which reports on a biodegradable polymeric matrix for containing a biological macromolecule. The polymeric matrix may be used to achieve sustained release of the agent in a subject. In some embodiments, an agent described herein may be encapsulated or dispersed within a biocompatible, preferably biodegradable polymeric matrix. The polymeric matrix may be in the form of a microparticle such as a microsphere (wherein the agent is dispersed throughout a solid polymeric matrix) or a microcapsule (wherein the agent is stored in the core of a polymeric shell). Other forms of polymeric matrix for containing the agent include films, coatings, gels, implants, and stents. The size and composition of the polymeric matrix device is selected to result in favorable release kinetics in the tissue into which the matrix device is implanted. The size of the polymeric matrix device further is selected according to the method of delivery which is to be used, typically injection into a tissue or administration of a suspension by aerosol into the nasal and/or pulmonary areas. The polymeric matrix composition can be selected to have both favorable degradation rates and also to be formed of a material which is bioadhesive, to further increase the effectiveness of transfer when the device is administered to a vascular, pulmonary, or other surface. The matrix composition also can be selected not to degrade, but rather, to release by diffusion over an extended period of time.

Both non-biodegradable and biodegradable polymeric matrices can be used to deliver the agents of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

In general, the agents may be delivered using the bio-erodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of non-biodegradable polymers include ethylene vinyl acetate, poly(meth)acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Bioadhesive polymers of particular interest include bioerodible hydrogels described by H. S. Sawhney, C. P. Pathak and J. A. Hubell in Macromolecules, 1993, 26, 581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Other delivery systems can include time-release, delayed release or sustained release delivery systems. Such systems can avoid repeated administrations of the peptide, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which the platelet reducing agent is contained in a form within a matrix such as those described in U.S. Pat. Nos. 4,452,775, 4,675,189, and 5,736,152 and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation. Liposomes, for example, which may comprise phospholipids or other lipids, are nontoxic, physiologically acceptable carriers that may be used in some embodiments. Liposomes can be prepared according to methods known to those skilled in the art. In some embodiments, for example, liposomes may be prepared as described in U.S. Pat. No. 4,522,811. Liposomes, including targeted liposomes, pegylated liposomes, and polymerized liposomes, are known in the art (see, e.g., Hansen C B, et al., Biochim Biophys Acta. 1239(2):133-44, 1995; Torchilin V P, et al., Biochim Biophys Acta, 1511(2):397-411, 2001; Ishida T, et al., FEBS Lett. 460(1):129-33, 1999). In some embodiments, a lipid-containing particle may be prepared as described in any of the following PCT application publications, or references therein: WO/2011/127255; WO/2010/080724; WO/2010/021865; WO/2010/014895; WO2010147655.

Use of a long-term sustained release implant may be particularly suitable for prophylactic treatment of subjects at risk of developing a recurrent cancer. Long-term release, as used herein, means that the implant is constructed and arranged to delivery therapeutic levels of the active agent for at least 30 days, and preferably 60 days. Long-term sustained release implants are well-known to those of ordinary skill in the art and include some of the release systems described above.

In some embodiments, it may be advantageous to formulate oral or parenteral compositions in dosage unit form for ease of administration and uniformity of dosage. Unit dosage form as used herein refers to physically discrete units suited as unitary dosages for the subject to be treated; each unit containing a predetermined quantity of active compound calculated to produce the desired therapeutic effect in association with the required pharmaceutical carrier.

If desired, toxicity and therapeutic efficacy of an agent or combination of agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. In some embodiments, a compound that exhibits a high therapeutic index may be selected. The data obtained from cell culture assays and animal studies can be used in formulating a range of dosage for use in humans. The dosage of such compounds lies preferably within a range of circulating concentrations that include the ED50 with little or no toxicity. The dosage can vary within this range depending upon the dosage form employed and the route of administration utilized. For any compound used in a method of treatment, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of a relevant parameter, e.g., cancer cell growth or other symptoms) as determined in cell culture. Such information can be used to more accurately determine useful doses in humans. Levels in plasma can be measured, for example, by high performance liquid chromatography. In some embodiments a compound described herein is used at a dose that has been demonstrated to have acceptable safety in at least one clinical trial or is a dose that is an acceptable dose or within an acceptable dose range as specified on an FDA-approved label for the compound. In some embodiments a compound described herein is used at a dose described in a patent or patent application describing such compound.

Generally, treatment of a subject can include a single treatment or, in many cases, can include a series of treatments. A pharmaceutical composition can be administered at various intervals and over different periods of time as required, e.g., multiple times per day, daily, every other day, once or more a week for between about 1 to 10 weeks, between 2 to 8 weeks, between about 3 to 7 weeks, about 4, 5, or 6 weeks, etc. It will be appreciated that multiple cycles of administration may be performed. Numerous variations are possible. The skilled artisan will appreciate that certain factors can influence the dosage and timing required to effectively treat a subject, including but not limited to the severity of the disease or disorder, previous treatments, the general health and/or age of the subject, and other diseases present.

Screening Methods

Some aspects of the disclosure are related to a method of identifying a candidate agent that modulates the expression of MCART1 or the activity of a gene product of MCART1 in a mitochondria.

In some embodiments, the method comprises contacting the mitochondria with a test agent, measuring a level of NAD or a NAD precursor in the mitochondria, and identifying the agent as an inhibitor of expression of MCART1 or the activity of a gene product of MCART1 if the level of NAD or a NAD precursor in the mitochondria is lower than a reference level, or identifying the test agent as an agent that increases expression of MCART1 or the activity of a gene product of MCART1 if the level of NAD or a NAD precursor in the mitochondria is higher than a reference level. In some embodiments, the reference level is the level of NAD or a NAD precursor in mitochondria under equivalent conditions but not exposed to the test agent. Methods of detecting NAD and NAD precursor are known in the art and are not limited. In some embodiment, the method is performed in isolated mitochondria. In some embodiments, the mitochondria are present in one or more cells. In some embodiments, the one or more cells is contacted with the test agent. In some embodiments, the mitochondria are isolated from one or more cells after contact with the test agent.

In some embodiments, the method comprises contacting a solution containing NAD or a NAD precursor and liposomes comprising MCART1 protein with a test agent and measuring a level of NAD or NAD precursor in the liposome as compared to a level of NAD or NAD precursor in a control liposome not exposed to the test agent.

In some embodiments, the level of NAD or a NAD precursor in the liposome or mitochondria is detected via fluorescence. In some embodiments, the liposome or mitochondria is capable of converting NAD to NADH and fluorescence of NADH is detected. In some embodiments, increases and decreases in expression of MCART1 are determined by modifying a cell to express MCART1 with a detectable tag. In some embodiments, the detectable tag is a fluorescent tag.

In some embodiments wherein the test agent is identified as an inhibitor of MCART1 expression or activity, the test agent is further contacted with a cell in the presence of a NAD transporter that is not MCART1 (e.g., NDT1 or MCART2) and determining if the NAD transporter reduces or eliminates a reduction in mitochondrial NAD levels caused by the test agent.

In some embodiments, an inhibitor of MCART1 protein activity (e.g., an expression product of the MCART1 gene) is identified by contacting a cell (e.g., cancer cell) relying upon OXPHOS for ATP production with a test agent and comparing growth or proliferation of the cell to a control cell not contacted with the agent, wherein the agent is identified as an inhibitor of MCART1 protein activity if the agent reduces growth or proliferation of the cell as compared to the control cell. In some embodiments, wherein the test agent is identified as an inhibitor of MCART1 activity, the test agent is further contacted with a cell in the presence of a NAD transporter that is not MCART1 (e.g., NDT1 or MCART2) and determining if the NAD transporter reduces or eliminates the inhibition in growth or activity.

In some embodiments the methods of screening test agents described herein further comprise contacting the identified MCART1 modulator with a test cell and measuring proliferation and/or survival of the contacted test cell as compared to a control cell not contacted with the identified modulator. In some embodiments the test cell and control cell are non-cancerous cells. In some embodiments the test cell and control cell are cancer cells. In some embodiments the methods may further comprise contacting the identified modulator, e.g., inhibitor, with a cancer cell and measuring proliferation and/or survival of the contacted cancer cell as compared to a non-cancerous cell not contacted with the identified modulator. In some embodiments a method comprises identifying an agent that selectively inhibits proliferation and/or survival of cancer cells as compared to non-cancerous cells.

In some embodiments, a method of screening one or more test agents to identify a modulator of MCART1 expression or activity comprises contacting a cell with the test agent and comparing expression or activity of MCART1 in the cell to a control. In some embodiments, the control is a control cell not contacted with the test agent. In some embodiments, the control is a predetermined level. In some embodiments, MCART1 mRNA or a protein level is determined. In some embodiments, a method of screening one or more test agents to identify a modulator of MCART2 expression or activity comprises contacting a cell with the test agent and comparing expression or activity of MCART2 in the cell to a control. In some embodiments, the control is a control cell not contacted with the test agent. In some embodiments, the control is a predetermined level. In some embodiments, MCART2 mRNA or a protein level is determined.

In some embodiments the methods comprise identifying an agent that modulates the expression or activity of MCART1 using a reporter assay. In some embodiments, a MCART1 promoter is operably linked to a sequence that encodes a reporter gene product (e.g., a luciferase enzyme). In some aspects the expression of the reporter gene is correlated with activity of the MCART1. In some aspects a cell containing the MCART1 promoter operably linked to a sequence that encodes the reporter gene product is contacted with a test agent and the expression of the reporter gene is measured. In some embodiments the test agent is identified as modulator of the MCART1 if the expression of the reporter gene product in the contacted cell is increased or decreased as compared to expression of a MCART1 promoter operably linked to a sequence that encodes a reporter gene product in a cell that is not contacted with a test agent.

In some embodiments the methods comprise identifying an agent that modulates the expression or activity of MCART2 using a reporter assay. In some embodiments, a MCART2 promoter is operably linked to a sequence that encodes a reporter gene product (e.g., a luciferase enzyme). In some aspects the expression of the reporter gene is correlated with activity of the MCART2. In some aspects a cell containing the MCART2 promoter operably linked to a sequence that encodes the reporter gene product is contacted with a test agent and the expression of the reporter gene is measured. In some embodiments the test agent is identified as modulator of the MCART2 if the expression of the reporter gene product in the contacted cell is increased or decreased as compared to expression of a MCART2 promoter operably linked to a sequence that encodes a reporter gene product in a cell that is not contacted with a test agent.

In some embodiments, a method of screening one or more test agents to identify a modulator of MCART1 expression or activity comprises a high-throughput transport assay (e.g., in vitro transport assay). In some aspects, an artificial membrane (e.g., a liposome) may be utilized. In other aspects a bacterial system may be utilized (e.g., Gram-negative bacteria such as E. coli or Gram-positive bacteria such as B. subtilis or Lactococcus lactis).

In certain embodiments of any method described herein, the survival or proliferation of cells, e.g., test cells and/or control cells, is determined by an assay selected from: a cell counting assay, a replication labeling assay, a cell membrane integrity assay, a cellular ATP-based viability assay, a mitochondrial reductase activity assay, a caspase activity assay, an Annexin V staining assay, a DNA content assay, a DNA degradation assay, and a nuclear fragmentation assay. Exemplary assays include BrdU, EdU, or H3-Thymidine incorporation assays; DNA content assays using a nucleic acid dye, such as Hoechst Dye, DAPI, actinomycin D, 7-aminoactinomycin D or propidium iodide; cellular metabolism assays such as AlamarBlue, MTT, XTT, and CellTitre Glo; nuclear fragmentation assays; cytoplasmic histone associated DNA fragmentation assay; PARP cleavage assay; TUNEL staining; and Annexin staining. In some embodiments, gene expression analysis (e.g., microarray, cDNA array, quantitative RT-PCR, RNAse protection assay, RNA-Seq) may be used to measure the expression of genes whose products mediate or are correlated with cell cycle, cell survival (or cell death, e.g., apoptosis), and/or cell proliferation, as an indication of the effect of an agent on cell viability or proliferation. Alternately or additionally, expression of proteins encoded by such genes may be measured. In other embodiments, the activity of a gene, such as those disclosed herein, can be assayed in a compound screen. In some embodiments, cells are modified to comprise an expression vector that includes a regulatory region of a gene whose products mediate or are correlated with cell cycle, cell survival (or cell death), and/or cell proliferation operably linked to a sequence that encodes a reporter gene product (e.g., a luciferase enzyme), wherein expression of the reporter gene is correlated with transcriptional activity of the gene. In such embodiments, assessment of reporter gene expression (e.g., luciferase activity) provides an indirect method for assessing cell survival or proliferation. Those of ordinary skill in the art are aware of genes whose products mediate or are correlated with cell cycle, cell survival (or cell death), and/or cell proliferation.

In some embodiments, the activity of an agent (e.g., a test agent) can be tested by contacting test cells and control cells that are in a co-culture. Co-cultures enable selective evaluation of the properties (e.g., survival or proliferation) of two or more populations of cells (e.g., test and control cells) in contact with an agent in a common growth chamber. Typically, each population of cells grown a co-culture will have an identifying characteristic that is detectable and distinct from an identifying characteristic of the other population(s) of cells in the co-culture. In some embodiments, the identifying characteristic comprises a level of expression of a fluorescent protein or other reporter protein or a protein expressed at the cell surface that could be detected using an antibody. Numerous fluorescent proteins are known in the art and may be used. Such proteins include, e.g., green, blue, yellow, red, orange, and cyan fluorescent proteins (FP). In some embodiments, test cells and control cells express different, distinguishable FPs, e.g., a red FP and a green FP, or other pairs of FPs that have different emission spectra. Other reporter proteins include, e.g., enzymes such as luciferase, beta-galactosidase, alkaline phosphatase, etc. However, other identifying characteristics known in the art may be suitable, provided that the identifying characteristic enables measurement (e.g., by FACS or other suitable assay method) of the level of survival or proliferation of each of the two or more populations of cells in the co-culture. A cell can be modified to have an identifying characteristic using methods known in the art, e.g., by introducing into the cell a nucleic acid construct encoding an FP (or other detectable protein) operably linked to a promoter. In some embodiments, a nucleic acid construct that encodes an RNAi agent that reduces expression of MCART1 and a nucleic acid construct that encodes a FP or other detectable protein are incorporated into the same vector. In some embodiments, they may be in different vectors. In some embodiments, the construct(s) may be integrated into the genome of the cell.

Compositions, e.g., co-cultures, comprising at least some test cells (e.g., between 1% and 99% test cells) and at least some control cells (e.g., between 1% and 99% control cells), are disclosed herein. In some embodiments the percentage of test cells is between 10% and 90%. In other embodiments the percentage of test cells is between 20% and 80%. In some embodiments the percentage of test cells is between 30% and 70%. In some embodiments the percentage of test cells is between 40% and 60%, e.g., about 50%. In some embodiments the composition further comprises a test agent.

In some embodiments, test cells and control cells are maintained in separate vessels (e.g., separate wells of a microwell plate) under substantially identical conditions.

Assay systems comprising test cells, control cells, and one or more test compounds, e.g., 10, 100, 1000, 10,000, or more test agents, wherein the cells and test agents are arranged in one or more vessels in a manner suitable for assessing effect of the test compound(s) on the cells, are aspects of the invention. Typically, the vessels contain a suitable tissue culture medium, and the test compounds are present in the tissue culture medium. One of skill in the art can select a medium and culture environment appropriate for culturing a particular cell type.

In various embodiments the number of test agents is at least 10; 100; 1000; 10,000; 100,000; 250,000; 500,000 or more. In some embodiments test agents are tested in individual vessels, e.g., individual wells of a multiwell plate (sometimes referred to as microwell or microtiter plate or dish). In some embodiments a multiwell plate of use in performing an assay or culturing or testing cells or agents has 6, 12, 24, 96, 384, or 1536 wells. Cells (test cells and/or control cells) can be contacted with one or more test agents for varying periods of time and/or at different concentrations. In certain embodiments cells are contacted with test agent(s) for between 1 hour and 20 days, e.g., for between 12 and 48 hours, between 48 hours and 5 days, e.g., about 3 days, between 2 and 5 days, between 5 days and 10 days, between 10 days and 20 days, or any intervening range or particular value. Cells can be contacted with a test agent during all or part of a culture period. Cells can be contacted transiently or continuously. Test agents can be added to culture media at the time of replenishing the media and/or between media changes. If desired, test agent can be removed prior to assessing growth and/or survival. In some embodiments a test agent is tested at 1, 2, 3, 5, 8, 10 or more concentrations. Concentrations of test agent may range, for example, between about 1 nM and about 100 μM. For example, concentrations 1 nM, 10 nM, 50 nM, 100 nM, 500 nM, 1 μM, 5 μM, 10 μM, 50 μM, 100 μM (or any subset of the foregoing) may be used.

In some embodiments of any aspect or embodiment in the present disclosure relating to cells, a population of cells, cell sample, or similar terms, the number of cells is between 10 and 10¹³ cells. In some embodiments the number of cells may be at least about 10³, 10⁴, 10⁵, 10⁶, 10⁷, 10⁸, 10⁹, 10¹⁰, 10¹¹, 10¹² cells, or more. In some embodiments, the number of cells is between 10⁵ and 10¹² cells, e.g., at least 10⁶, 10⁷, 10⁸, 10⁹, 10¹⁰, 10¹¹, up to about 10¹² or about 10¹³. In some embodiments a screen is performed using multiple populations of cells and/or is repeated multiple times. In some embodiments, the number of cells is between 10⁵ and 10¹² cells, e.g., at least 10⁶, 10⁷, 10⁸, 10⁹, 10¹⁰, 10¹¹, up to about 10¹². In some embodiments smaller numbers of cells are of use, e.g., between 1-10⁴ cells. In some embodiments a population of cells is contained in an individual vessel, e.g., a culture vessel such as a culture plate, flask, or well. In some embodiments a population of cells is contained in multiple vessels. In some embodiments two or more cell populations are pooled to form a larger population.

In some embodiments, each of one or more test cells is contacted with a different concentration of, and/or for a different duration with, a test agent than at least one other test cell; and/or each of the one or more control cells is contacted with a different concentration of, and/or for a different duration with, the test agent than at least one other control cell.

In some embodiments, a method may comprise generating a dose response curve for an agent, test cells, and/or control cells, wherein the dose response curve for test cells indicates the level of inhibition of survival or proliferation of the one or more test cells by the agent at a plurality of doses and wherein the dose response curve for control cells indicates the level of inhibition of survival or proliferation of the one or more control cells by the agent at a plurality of doses. In some embodiments, a method may comprise generating a dose response curve that indicates the relative level of inhibition of survival or proliferation of test cells versus control cells at a plurality of doses.

In some embodiments, a method may further comprise determining (e.g., by analyzing a dose response curve) an IC50, EC50, or both, for an agent. In some embodiments an agent is identified for which the IC50 value, the EC50 value, or both, for the agent on the one or more test cells is statistically significantly less than the IC50 value for the agent on the one or more control cells. In some embodiments, an agent is identified for which the IC50 value, the EC50 value, or both, for the agent on the one or more test cells is statistically significantly less than the EC50 value for the agent on the one or more control cells.

In some embodiments, a high throughput screen (HTS) is performed. A high throughput screen can utilize cell-free or cell-based assays. High throughput screens often involve testing large numbers of compounds with high efficiency, e.g., in parallel. For example, tens or hundreds of thousands of compounds can be routinely screened in short periods of time, e.g., hours to days. Often such screening is performed in multiwell plates containing, at least 96 wells or other vessels in which multiple physically separated cavities or depressions are present in a substrate. High throughput screens often involve use of automation, e.g., for liquid handling, imaging, data acquisition and processing, etc. Certain general principles and techniques that may be applied in embodiments of a HTS of the present invention are described in Macarrón & Hertzberg R P. Design and implementation of high-throughput screening assays. Methods Mol Biol., 565:1-32, 2009 and/or An W F & Tolliday N J., Introduction: cell-based assays for high-throughput screening. Methods Mol Biol. 486:1-12, 2009, and/or references in either of these. Useful methods are also disclosed in High Throughput Screening: Methods and Protocols (Methods in Molecular Biology) by William P. Janzen (2002) and High-Throughput Screening in Drug Discovery (Methods and Principles in Medicinal Chemistry) (2006) by Jorg Hüser.

The term “hit” generally refers to an agent that achieves an effect of interest in a screen or assay, e.g., an agent that has at least a predetermined level of modulating effect on cell survival, cell proliferation, gene expression, protein activity, or other parameter of interest being measured in the screen or assay. Test agents that are identified as hits in a screen may be selected for further testing, development, or modification. In some embodiments a test agent is retested using the same assay or different assays. For example, a candidate anticancer agent may be tested against multiple different cancer cell lines or in an in vivo tumor model to determine its effect on cancer cell survival or proliferation, tumor growth, etc. Additional amounts of the test agent may be synthesized or otherwise obtained, if desired. Physical testing or computational approaches can be used to determine or predict one or more physicochemical, pharmacokinetic and/or pharmacodynamic properties of compounds identified in a screen. For example, solubility, absorption, distribution, metabolism, and excretion (ADME) parameters can be experimentally determined or predicted. Such information can be used, e.g., to select hits for further testing, development, or modification. For example, small molecules having characteristics typical of “drug-like” molecules can be selected and/or small molecules having one or more unfavorable characteristics can be avoided or modified to reduce or eliminated such unfavorable characteristic(s).

Additional compounds, e.g., analogs, that have a desired activity can be identified or designed based on compounds identified in a screen. In some embodiments structures of hit compounds are examined to identify a pharmacophore, which can be used to design additional compounds. An additional compound may, for example, have one or more altered, e.g., improved, physicochemical, pharmacokinetic (e.g., absorption, distribution, metabolism and/or excretion) and/or pharmacodynamic properties as compared with an initial hit or may have approximately the same properties but a different structure. For example, a compound may have higher affinity for the molecular target of interest, lower affinity for a non-target molecule, greater solubility (e.g., increased aqueous solubility), increased stability, increased bioavailability, oral bioavailability, and/or reduced side effect(s), modified onset of therapeutic action and/or duration of effect. An improved property is generally a property that renders a compound more readily usable or more useful for one or more intended uses. Improvement can be accomplished through empirical modification of the hit structure (e.g., synthesizing compounds with related structures and testing them in cell-free or cell-based assays or in non-human animals) and/or using computational approaches. Such modification can make use of established principles of medicinal chemistry to predictably alter one or more properties. An analog that has one or more improved properties may be identified and used in a composition or method described herein. In some embodiments a molecular target of a hit compound is identified or known. In some embodiments, additional compounds that act on the same molecular target may be identified empirically (e.g., through screening a compound library) or designed.

Data or results from testing an agent or performing a screen may be stored or electronically transmitted. Such information may be stored on a tangible medium, which may be a computer-readable medium, paper, etc. In some embodiments a method of identifying or testing an agent comprises storing and/or electronically transmitting information indicating that a test agent has one or more propert(ies) of interest or indicating that a test agent is a “hit” in a particular screen, or indicating the particular result achieved using a test agent. A list of hits from a screen may be generated and stored or transmitted. Hits may be ranked or divided into two or more groups based on activity, structural similarity, or other characteristics

Once a candidate agent is identified, additional agents, e.g., analogs, may be generated based on it. An additional agent, may, for example, have increased cancer cell uptake, increased potency, increased stability, greater solubility, or any improved property. In some embodiments a labeled form of the agent is generated. The labeled agent may be used, e.g., to directly measure binding of an agent to a molecular target in a cell. In some embodiments, a molecular target of an agent identified as described herein may be identified. An agent may be used as an affinity reagent to isolate a molecular target. An assay to identify the molecular target, e.g., using methods such as mass spectrometry, may be performed. Once a molecular target is identified, one or more additional screens maybe performed to identify agents that act specifically on that target.

Any of a wide variety of agents may be used as a test agent in various embodiments. For example, a test agent may be a small molecule, polypeptide, peptide, amino acid, nucleic acid, oligonucleotide, lipid, carbohydrate, or hybrid molecule. In some embodiments a nucleic acid used as a test agent comprises a siRNA, shRNA, antisense oligonucleotide, aptamer, or random oligonucleotide. In some embodiments a test agent is cell permeable or provided in a form or with an appropriate carrier or vector to allow it to enter cells.

Agents can be obtained from natural sources or produced synthetically. Agents may be at least partially pure or may be present in extracts or other types of mixtures. Extracts or fractions thereof can be produced from, e.g., plants, animals, microorganisms, marine organisms, fermentation broths (e.g., soil, bacterial or fungal fermentation broths), etc. In some embodiments, a compound collection (“library”) is tested. A compound library may comprise natural products and/or compounds generated using non-directed or directed synthetic organic chemistry. In some embodiments a library is a small molecule library, peptide library, peptoid library, cDNA library, oligonucleotide library, or display library (e.g., a phage display library). In some embodiments a library comprises agents of two or more of the foregoing types. In some embodiments oligonucleotides in an oligonucleotide library comprise siRNAs, shRNAs, antisense oligonucleotides, aptamers, or random oligonucleotides.

A library may comprise, e.g., between 100 and 500,000 compounds, or more. In some embodiments a library comprises at least 10,000, at least 50,000, at least 100,000, or at least 250,000 compounds. In some embodiments compounds of a compound library are arrayed in multiwell plates. They may be dissolved in a solvent (e.g., DMSO) or provided in dry form, e.g., as a powder or solid. Collections of synthetic, semi-synthetic, and/or naturally occurring compounds may be tested. Compound libraries can comprise structurally related, structurally diverse, or structurally unrelated compounds. Compounds may be artificial (having a structure invented by man and not found in nature) or naturally occurring. In some embodiments compounds that have been identified as “hits” or “leads” in a drug discovery program and/or analogs thereof. In some embodiments a library may be focused (e.g., composed primarily of compounds having the same core structure, derived from the same precursor, or having at least one biochemical activity in common). Compound libraries are available from a number of commercial vendors such as Tocris BioScience, Nanosyn, BioFocus, and from government entities such as the U.S. National Institutes of Health (NIH). In some embodiments a test agent is not an agent that is found in a cell culture medium known or used in the art, e.g., for culturing vertebrate, e.g., mammalian cells, e.g., an agent provided for purposes of culturing the cells. In some embodiments, if the agent is one that is found in a cell culture medium known or used in the art, the agent may be used at a different, e.g., higher, concentration when used as a test agent in a method or composition described herein.

Specific examples of certain aspects of the inventions disclosed herein are set forth below in the Examples.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more nucleic acids, polypeptides, cells, species or types of organism, disorders, subjects, or combinations thereof, can be excluded.

Where the claims or description relate to a composition of matter, e.g., a nucleic acid, polypeptide, cell, or non-human transgenic animal, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXAMPLES

Nicotinamide adenine dinucleotide (NAD) is an essential cofactor in redox metabolism and metabolic signaling. As cofactors of metabolic enzymes, NAD⁺ and its reduced form, NADH, function in redox reactions in central metabolic pathways including glycolysis, TCA cycle, oxidative phosphorylation, one-carbon metabolism and control the direction of flux through these pathways. As a cosubstrate of sirtuins and poly-(ADP-ribose) polymerases, NAD⁺ mediates posttranslational modification of metabolic enzymes, DNA repair and chromatin-modifying proteins, and other factors involved in stress response and signaling pathways that connect metabolism to different physiological responses (1). NAD levels decline in aging and administration of NAD precursors is currently being tested in the clinic as a measure to prevent age-associated diseases ((2, 3)).

Despite its importance for many cellular processes, the compartmentalization of NAD synthesis and its transport remain poorly understood. Up to ˜70% of cellular NAD has been reported to be present within mitochondria (4), where it serves as an electron carrier connecting fuel oxidation in the TCA cycle with the electron transport chain (ETC) and thus ATP synthesis. Strikingly, if and how NAD is imported into mitochondria in higher eukaryotes is not known. While in yeast and plants, mitochondrial and chloroplast NAD⁺ transporters have been identified (5, 6), the functional ortholog in animals remains elusive.

MCART1 is an Inner Mitochondrial Membrane Protein Required for Mitochondrial Respiration

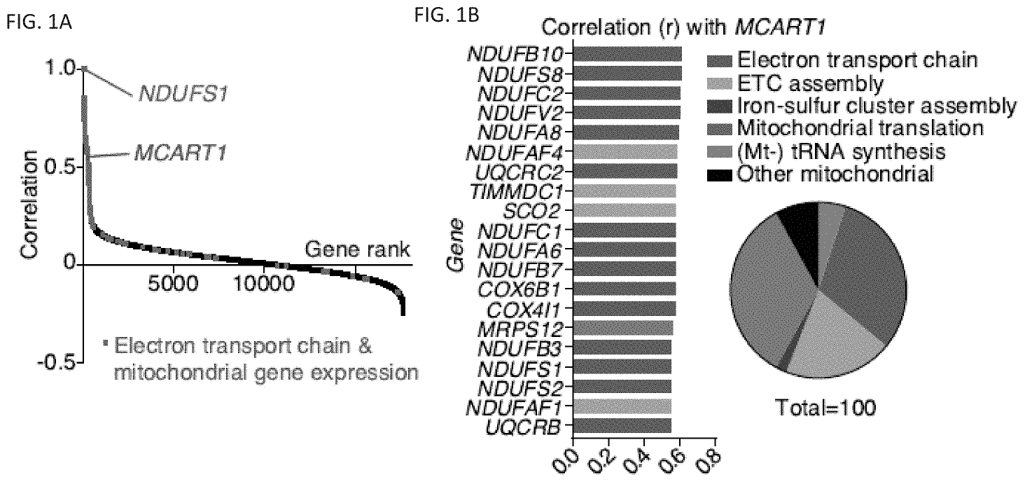

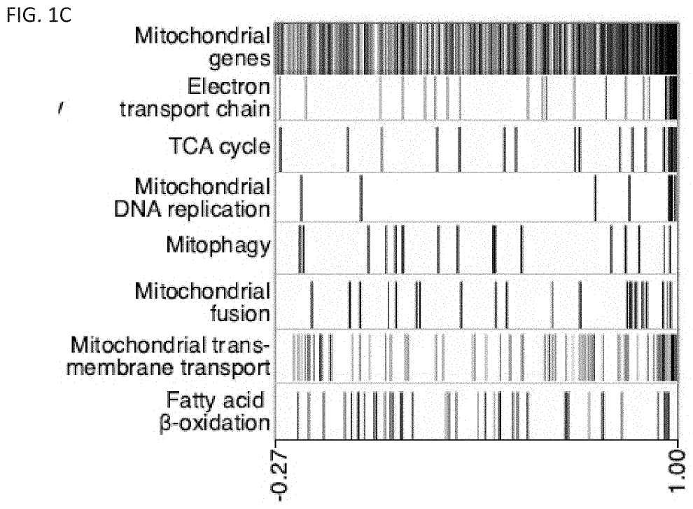

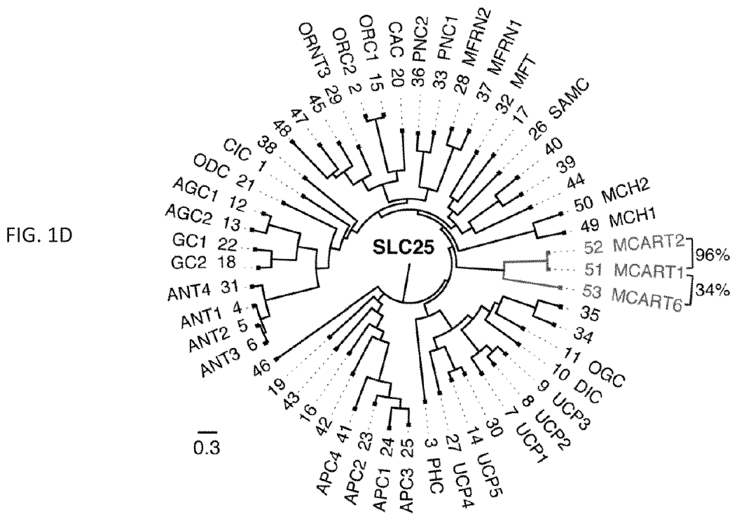

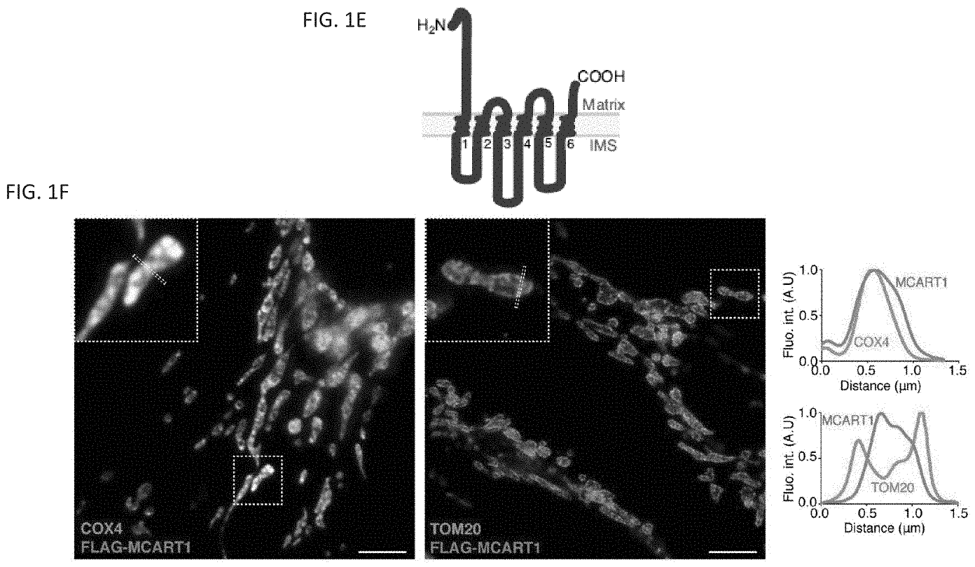

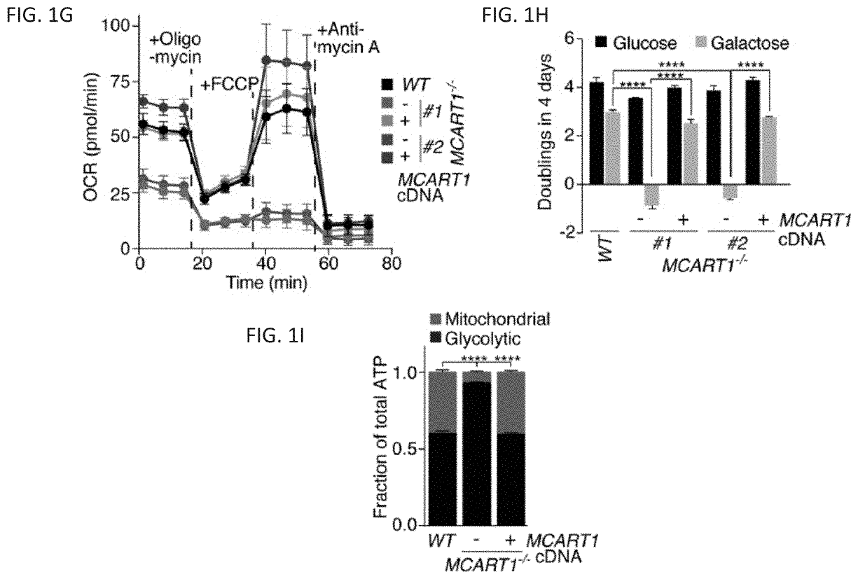

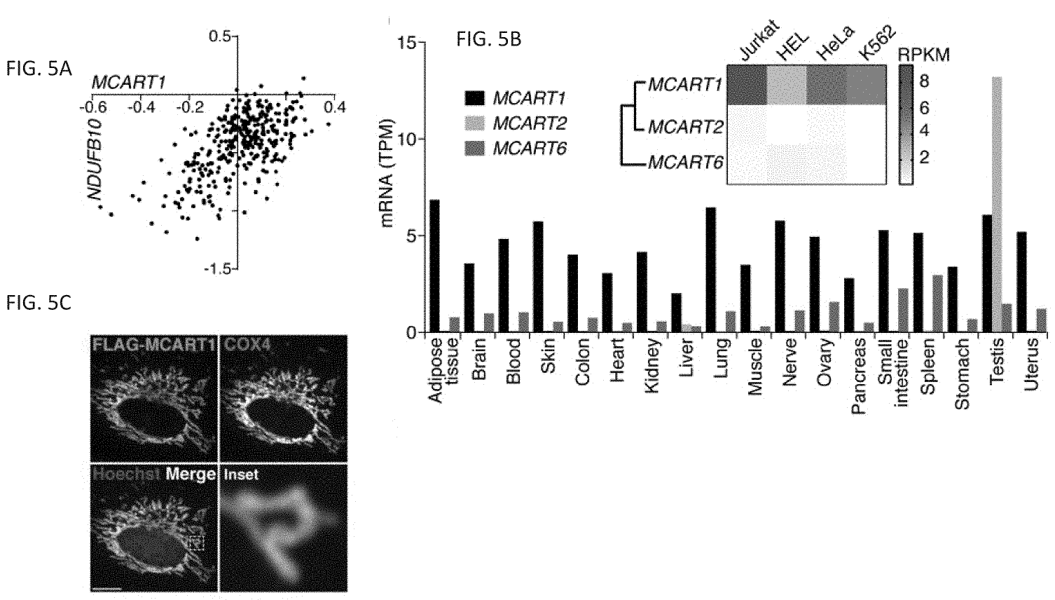

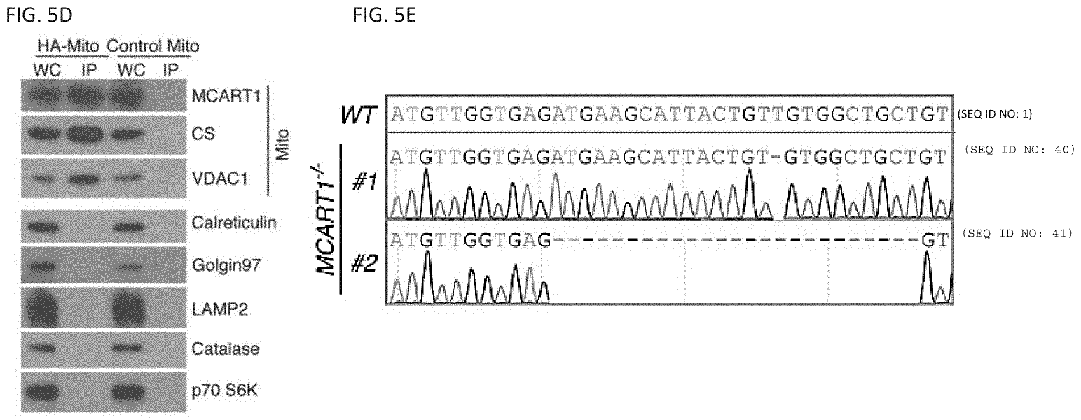

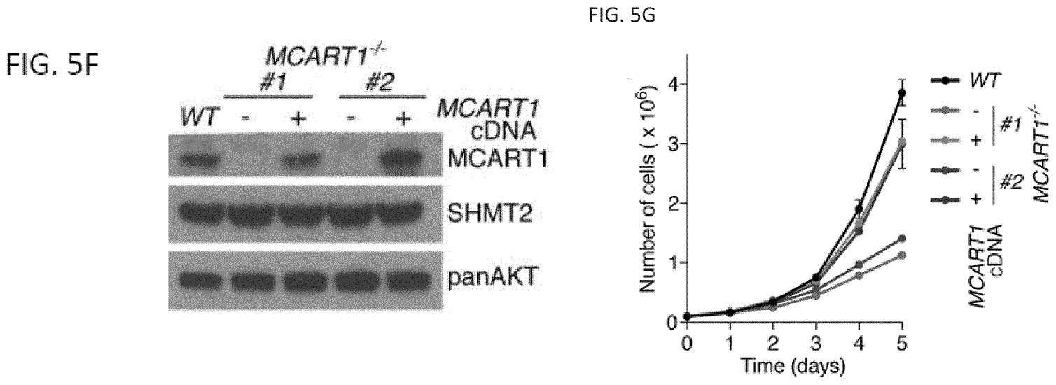

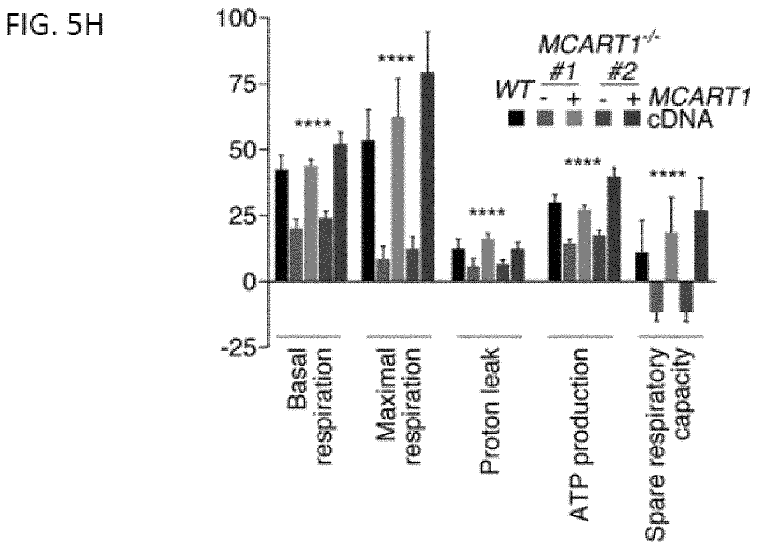

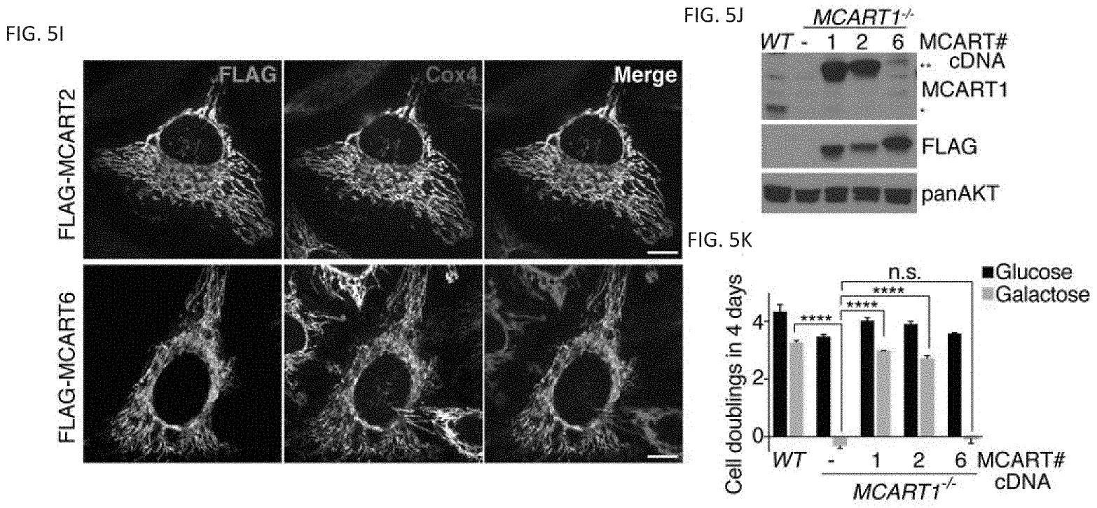

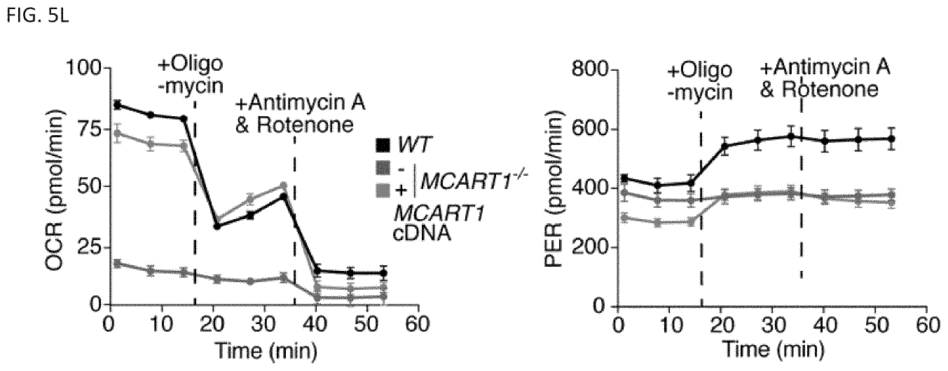

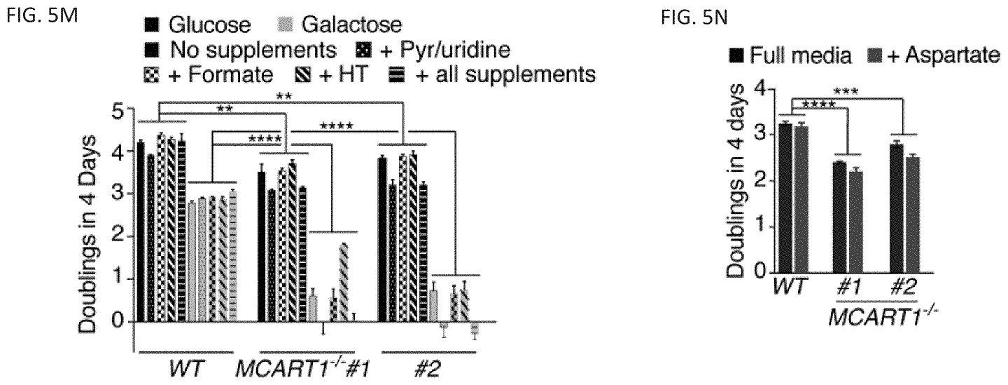

To identify novel genes required for mitochondrial respiration, a previously described approach (7) was used to mine the Achilles dataset containing gene essentiality scores from 341 cell lines (8) for genes that were co-essential with the nuclear-encoded core component of respiratory complex I of the ETC, NDUFS1. This analysis identified a cluster of ˜400 co-essential genes, most of which were previously annotated as encoding mitochondrially-localized proteins, including components of or assembly factors for the ETC, and components of the mitochondrial translation machinery, whose main function is to synthesize the 13 mitochondrially-encoded components of the ETC (FIG. 1A). Embedded in this gene cluster, a thus far unstudied gene, MCART1 (SLC25A51) (FIG. 1A) was identified. The top 20 genes co-essential with MCART1 are involved in ETC function or mitochondrial translation (FIG. 1B and FIG. 5A), and this gene cluster is enriched specifically for genes that impact ETC function or related processes, such as the TCA cycle, but not other mitochondrial processes, such as mitophagy or mitochondrial fusion (FIG. 1C).

MCART1 is an unstudied member of the SLC25 family of mitochondrial triple repeat carriers comprising 53 members in humans (FIG. 1D). Two other SLC25 family members, MCART2 (SLC25A52) and MCART6 (SLC25A53), are particularly similar to MCART1, with MCART2 having a striking 96% sequence identity (NCBI blast). However, while MCART1 is expressed at considerable levels across tissues and commonly used cell lines, the expression of MCART6 is generally lower and that of MCART2 appears restricted to the testis (FIGS. 5B and 5C). As other members of the SLC25 family, the MCART1 protein is predicted to have six transmembrane domains, with both N- and C-termini localized in the mitochondrial matrix (FIG. 1E; analysis with Protter (9)). As expected, FLAG-tagged MCART1 co-localized with the inner mitochondrial membrane protein COX4 in HeLa cells (FIG. 5C), and endogenous MCART1 was enriched in mitochondria purified from Jurkat cells (FIG. 5D). Super-resolution microscopy revealed that MCART1 localizes to the inner mitochondrial membrane, consistent with a function in transporting a metabolite into mitochondria (FIG. 1F).

To test whether MCART1 has a role in oxidative phosphorylation, MCART1 in human Jurkat leukemic T-cells was deleted using CRISPR-Cas9. Two clones with complete deletion of MCART1 were chosen for downstream phenotypic analysis (FIGS. 5E and 5F; where not indicated, clone #1 was used in experiments). MCART1-null cells had a strong proliferation defect in full media at early passages (FIG. 5G), and increased acidification of the culture media was observed despite their reduced proliferation suggesting a defect in mitochondrial function. Indeed, cells lacking MCART1 had a dramatically reduced oxygen consumption rate, stemming from reduced basal and maximal respiration, spare respiratory capacity, proton leak and ATP production as measured by Seahorse extracellular flux analysis (FIG. 1G, FIG. 5H). MCART1-null cells were also unable to proliferate in media containing galactose instead of glucose as a carbon source, conditions under which cells must generate ATP from mitochondrial respiration (FIG. 1H). Indeed, MCART1-null cells were defective in mitochondrial ATP production and instead generated the vast majority of their ATP from cytosolic glycolysis (FIG. 1I and FIGS. 5H and 5L). Importantly, re-expression of a guide-resistant cDNA for MCART1 reversed all of these defects (FIG. 1G-H, and FIGS. 5G, 5H, 5L). Expression of MCART2, the closest MCART1 homolog, but not of MCART6, rescued proliferation on galactose (FIG. 5I-K). Interestingly, the proliferation defect of MCART1-null cells was not rescued by the addition to the media of metabolites known to bypass different aspects of mitochondrial function, such as pyruvate and uridine, formate, hypoxanthine-thymidine, or aspartate even in cells expressing a plasma membrane aspartate transporter (10-15)(FIGS. 5M and 5N). Inability of these metabolites to rescue the proliferation of MCART1-null cells suggests that ATP levels are limiting for proliferation, as described previously for mitochondrial dysfunction caused by loss of ETC components (16).

Loss of MCART1 Results in Defects in Mitochondrial Metabolism and ETC Complex I Activity without Affecting Mitochondrial Integrity

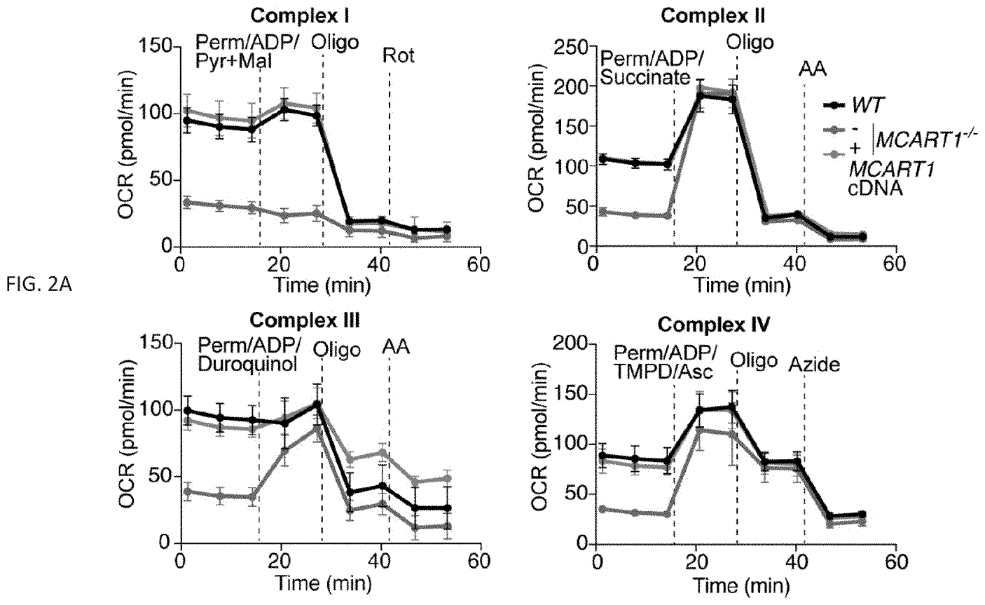

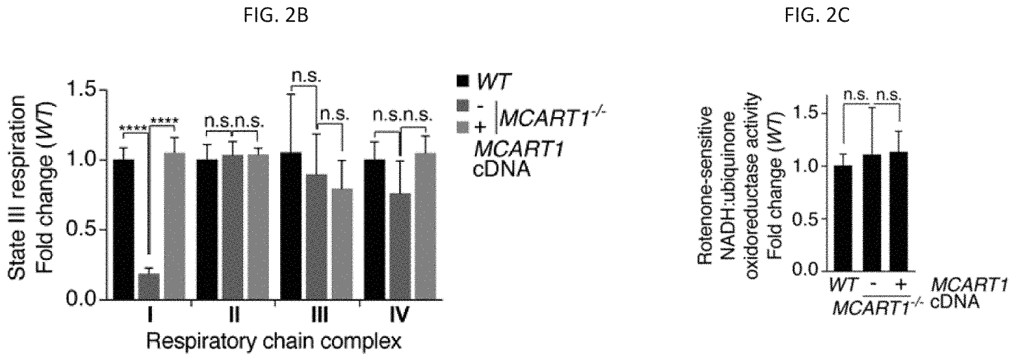

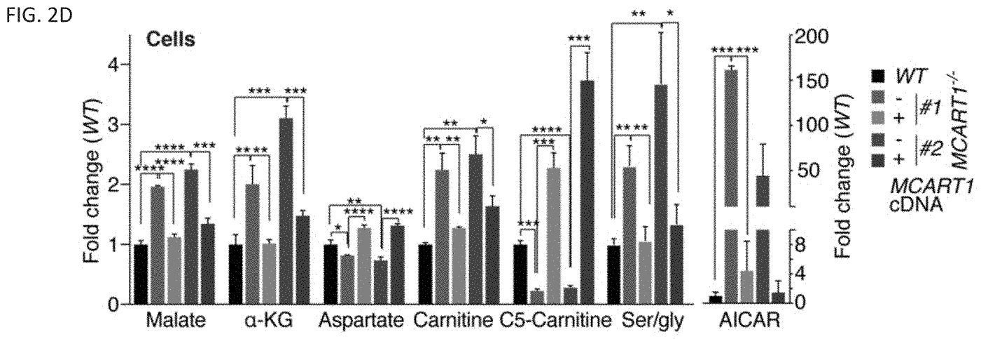

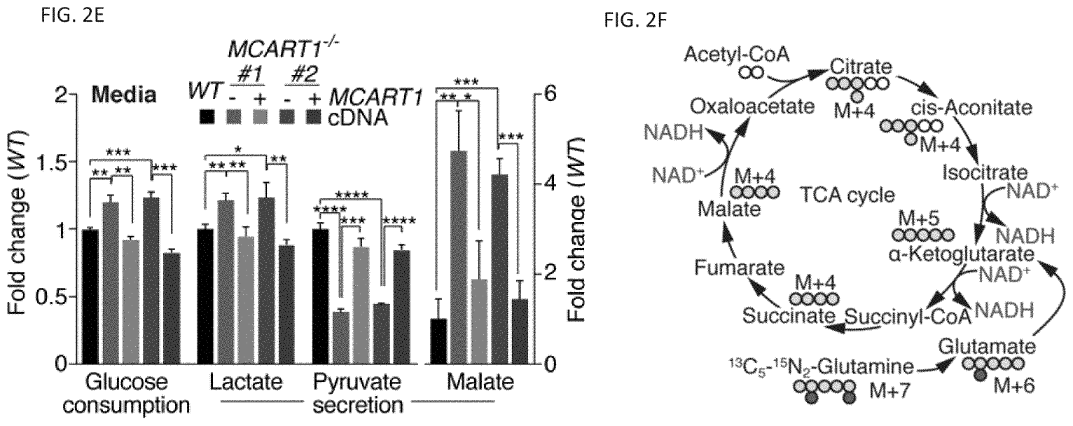

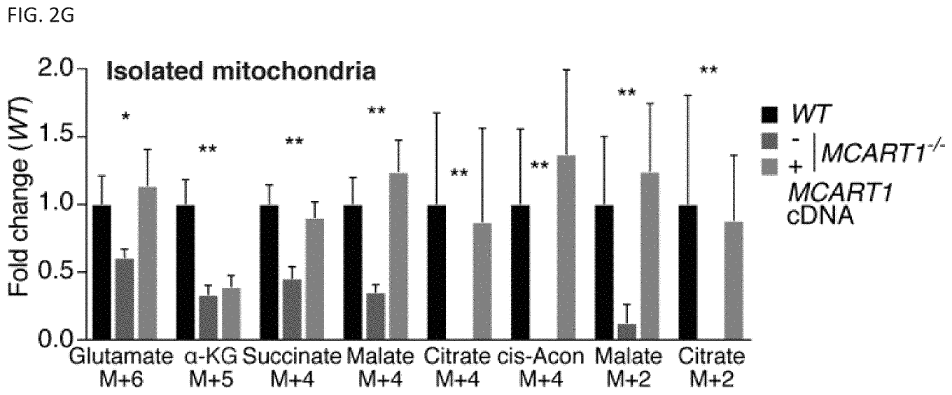

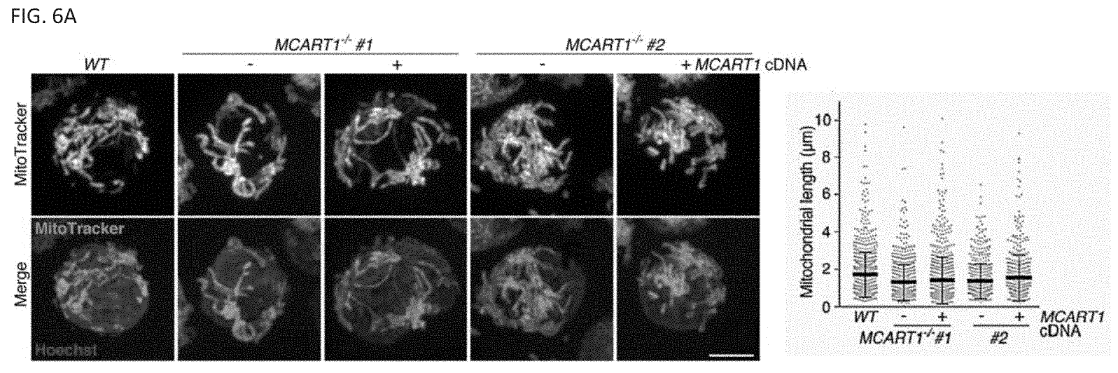

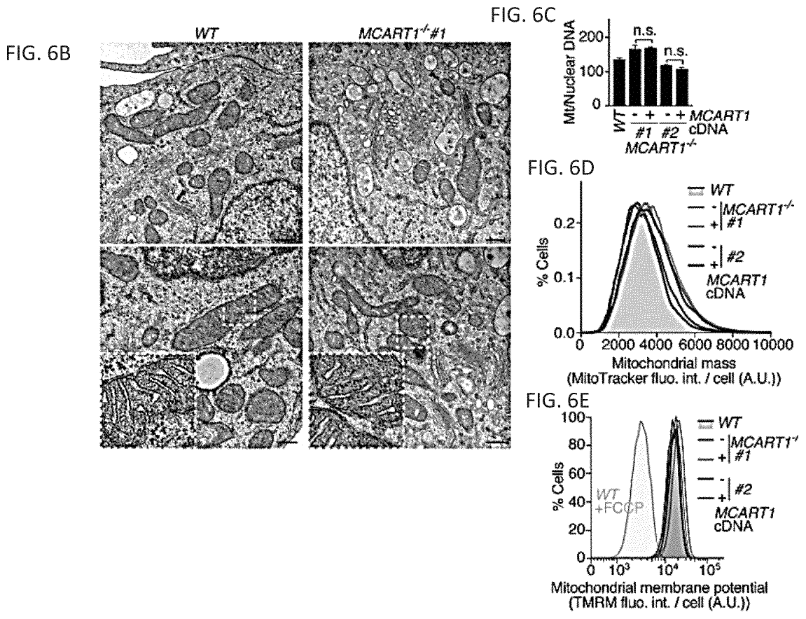

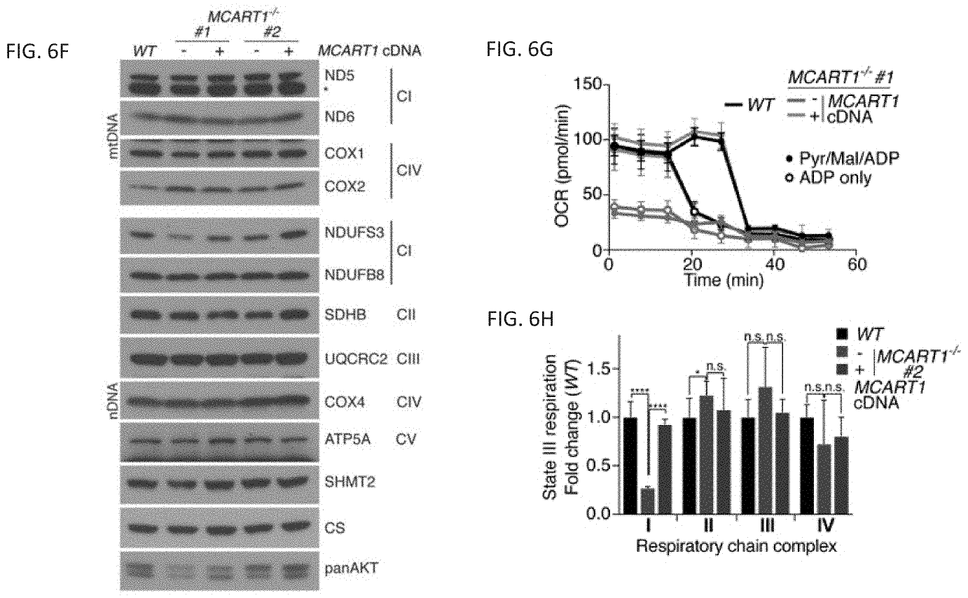

Mitochondrial dysfunction and respiratory defects are often due to defects in mitochondrial replication, translation or structural integrity leading to loss of respiratory chain complexes. However, loss of MCART1 did not result in changes in mitochondrial or cristae morphology nor mitochondrial DNA or mass (FIG. 6A-D). Furthermore, the mitochondrial membrane potential and levels of mitochondrially as well nuclear-encoded mitochondrial proteins were unaffected (FIGS. 6E and 6F). To test whether the ETC generally or a specific respiratory complex was affected in MCART1 -null cells, the activity of each respiratory chain complex was measured by providing (artificial) substrates for each to permeabilized cells and analyzed oxygen consumption rate by Seahorse extracellular flux analysis. Remarkably, complex I activity was ablated in MCART1-null cells, while that of other respiratory chain complexes was comparable to that in wild-type cells, or cells re-expressing MCART1 (FIGS. 2A and 2B, FIGS. 6G and 6H). When substrates were added directly to mitochondrial lysates, the NADH:ubiquinone oxidoreductase activity of complex I in MCART1-null cells did not differ from that in wild-type cells, arguing that complex I levels, assembly or function itself were not perturbed (FIG. 2C). Collectively, these data suggested that the mitochondrial dysfunction caused by MCART1 loss was most likely caused by loss of a metabolite in the mitochondria with a specific role in complex I of the ETC.

To understand how mitochondrial metabolism was altered upon loss of MCART1, LC-MS-based metabolomics analyses was used. A number of mitochondria-derived metabolites, such as TCA cycle intermediates, the fatty acid carrier carnitine and the purine synthesis intermediate 5-Aminoimidazole-4-carboxamide ribonucleotide (AICAR), were dramatically changed in MCART1-null cells (FIG. 2D). MCART1-null cells also consumed glucose and secreted lactate and malate at higher rates than wild-type cells, and secreted less pyruvate to the media (FIG. 2E). These changes in intracellular and media metabolites indicated extensive alterations in TCA cycle and one-carbon flux, fatty acid/branched chain amino acid-oxidation and ETC function, all of which are major mitochondrial metabolic pathways, and are consistent with a switch to aerobic glycolysis in MCART1-null cells. To assess the capacity for mitochondria to catabolize nutrients, TCA cycle flux was probed in mitochondria specifically by incubating isolated mitochondria with stably isotope-labeled ¹³C₅-¹⁵N₂-glutamine (M+7) and newly synthesized (labeled) TCA cycle metabolites were measured by LC-MS (FIG. 2F). The use of isolated mitochondria in this assay is critical because some TCA cycle intermediates can also be synthesized in the cytosol or nucleus (17-19). Despite significant glutaminolysis still occurring as assessed by M+6 glutamine levels, detectable TCA cycle intermediates produced in the first (M+5, M+4) and second rounds (M+2) of the TCA cycle were dramatically decreased or undetectable when MCART1-null mitochondria were used in the reaction (FIG. 2G). Of note, when glutamine tracing was performed in whole cells no decrease in the production of TCA cycle metabolites was observed indicating an increased activity, perhaps due to compensation, of cytosolic isoenzymes (FIG. 6I).

Together these results suggested that major metabolic pathways are perturbed in mitochondria of MCART1 -null cells.

Depletion of NAD⁺ and NADH in Mitochondria from MCART1-Null Cells

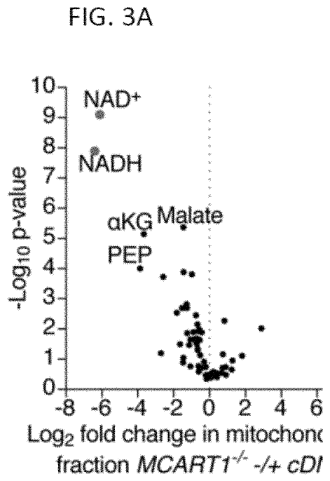

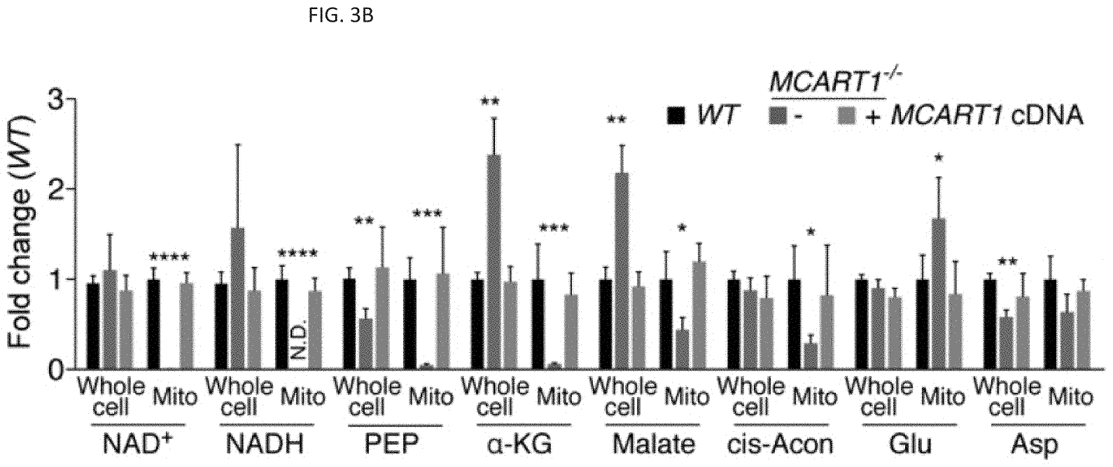

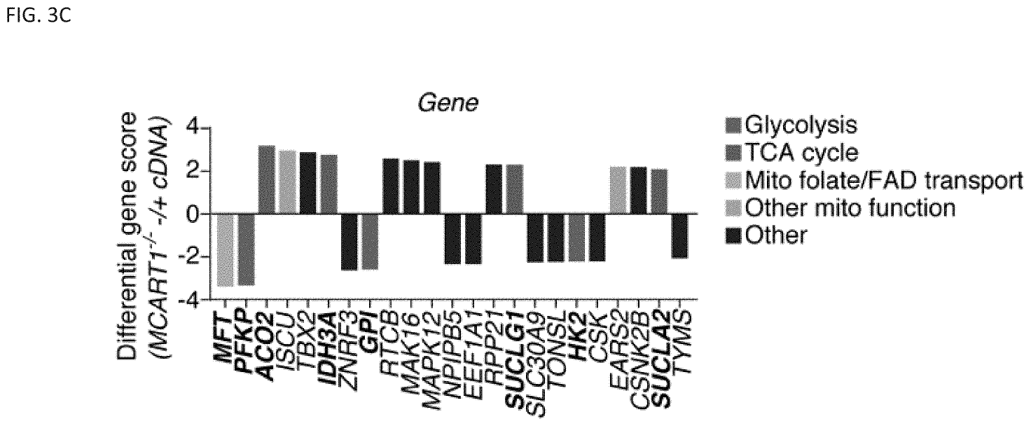

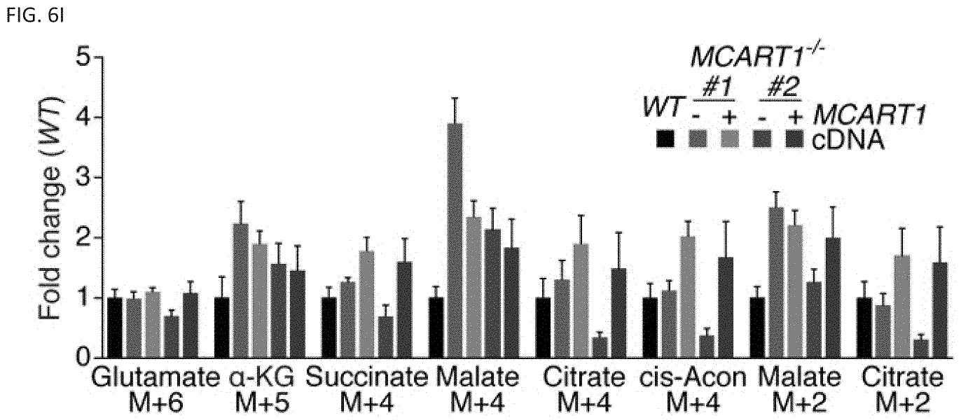

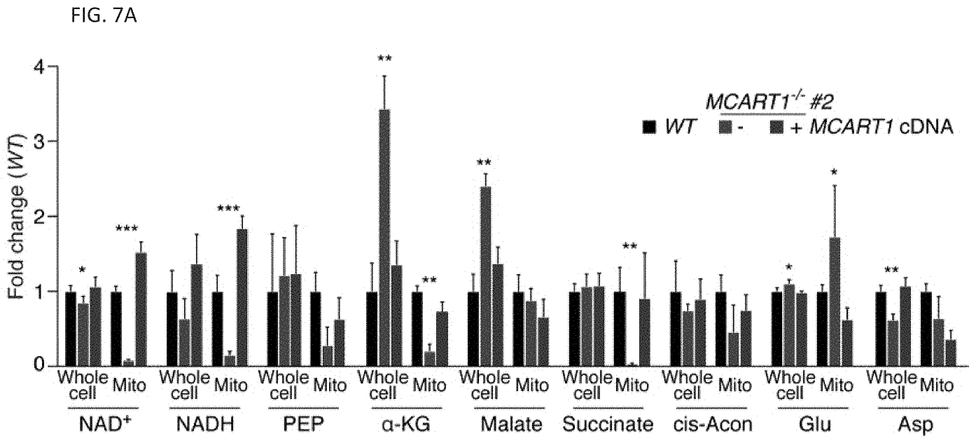

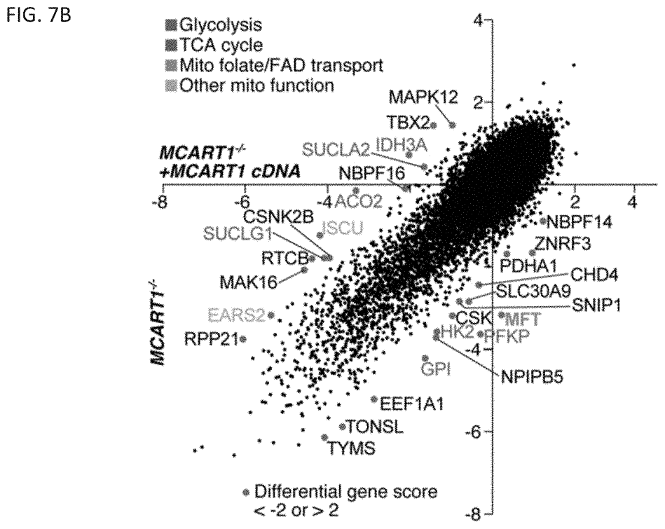

To understand how loss of MCART1 affects metabolism in mitochondria specifically, mitochondria were isolated using the Mito-IP approach and metabolites were broadly profiled by LC-MS, allowing detection of the mitochondrial metabolites whose levels most dramatically change upon loss of MCART1 (20). Strikingly, the largest difference between mitochondria with and without MCART1 was in the dinucleotide NAD in both its oxidized (NAD⁺) as well as reduced form (NADH; FIG. 3A). NAD⁺ and NADH were undetectable in mitochondria of MCART1-null cells, while their whole cell levels were unaffected (FIG. 3B, FIG. 7A). Decreases in the TCA cycle intermediates cis-aconitate, alpha-ketoglutarate and malate were specifically observed in mitochondria, as well as an overall decrease in phosphoenolpyruvate, which is generated from the TCA cycle intermediate oxaloacetate, consistent with TCA cycle flux analysis. Glutamate was increased, consistent with repressed TCA cycle anaplerosis, and other mitochondrial metabolites were only slightly or not significantly changed (FIG. 3B, FIG. 7A).

The NAD⁺/NADH redox pair is a critical cofactor in mitochondrial metabolism and acts as an electron carrier feeding into ETC complex I. Thus, the specific loss of complex I activity and the metabolite changes observed in MCART1-null cells are consistent with a loss of mitochondrial NAD and together these results suggested MCART1 functions in the uptake of NAD or an NAD precursor into mitochondria. In subsequent metabolite profiling experiments, a metabolite extraction method optimized for preserving NAD-related metabolites was used (adapted from (21)).

To corroborate this possible function of MCART1 in an unbiased way, genes synthetically lethal with MCART1 were identified using a negative selection-Cas9 screen. Consistent with previous results, several genes involved in glycolysis were selectively essential in cells lacking MCART1, while control cells re-expressing the MCART1 cDNA depended more on TCA cycle enzymes (FIG. 3C, FIG. 7B). Notably, the mitochondrial folate carrier MFT/SCL25A32, which transports the redox cofactor FAD and folates into mitochondria (22-26), was the most selectively essential gene in MCART1-null cells. This could be explained by the notion that depletion of one redox cofactor (NAD) from mitochondria upon MCART1 loss results in increased dependence on another (FAD).

A Yeast Mitochondrial NAD⁺ Transporter but not a Predicted Substrate-Binding Mutant of MCART1 Rescues Loss of MCART1

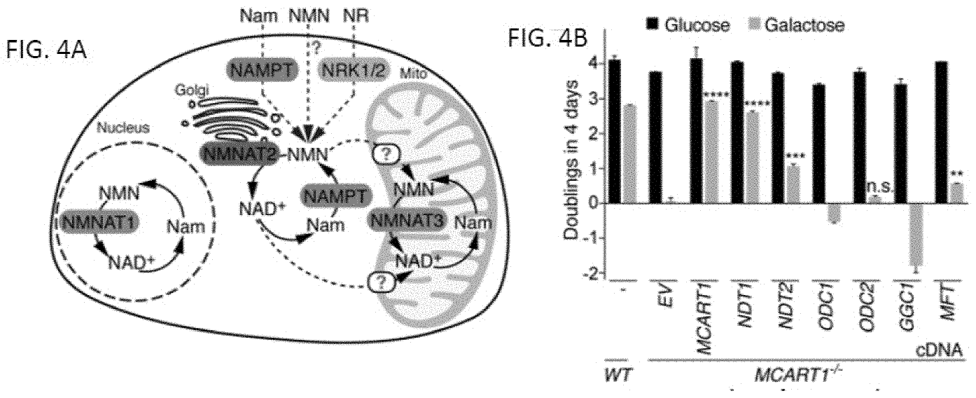

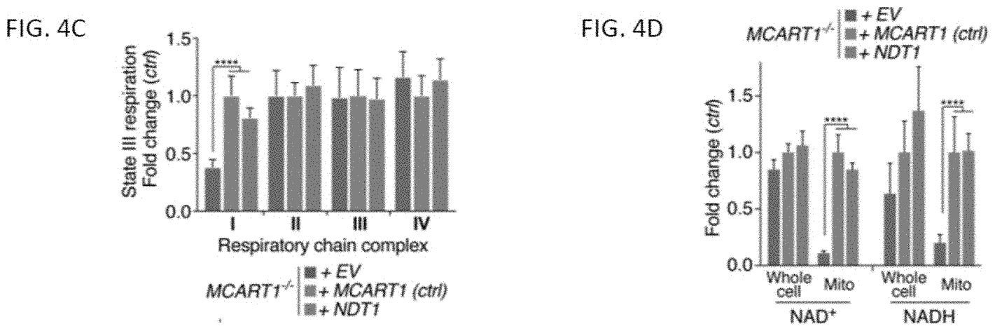

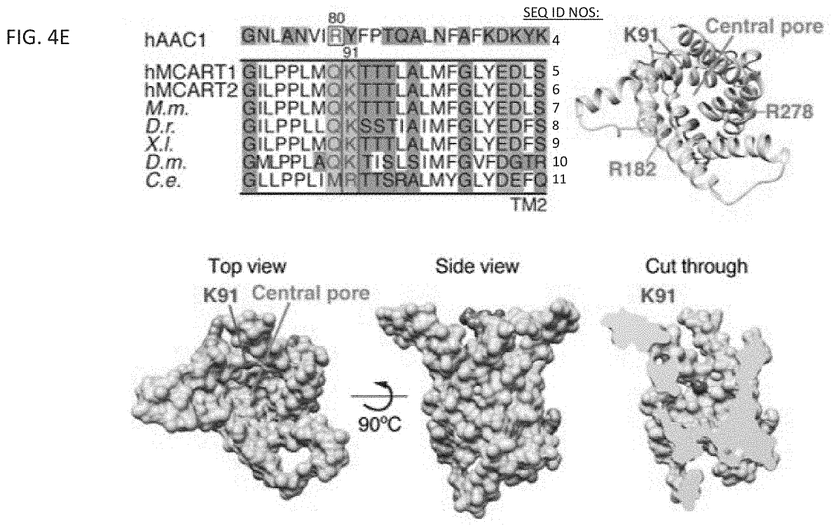

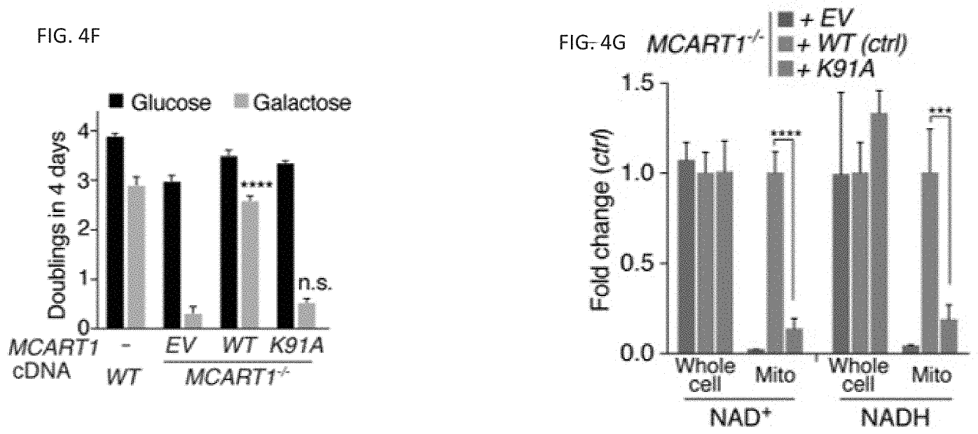

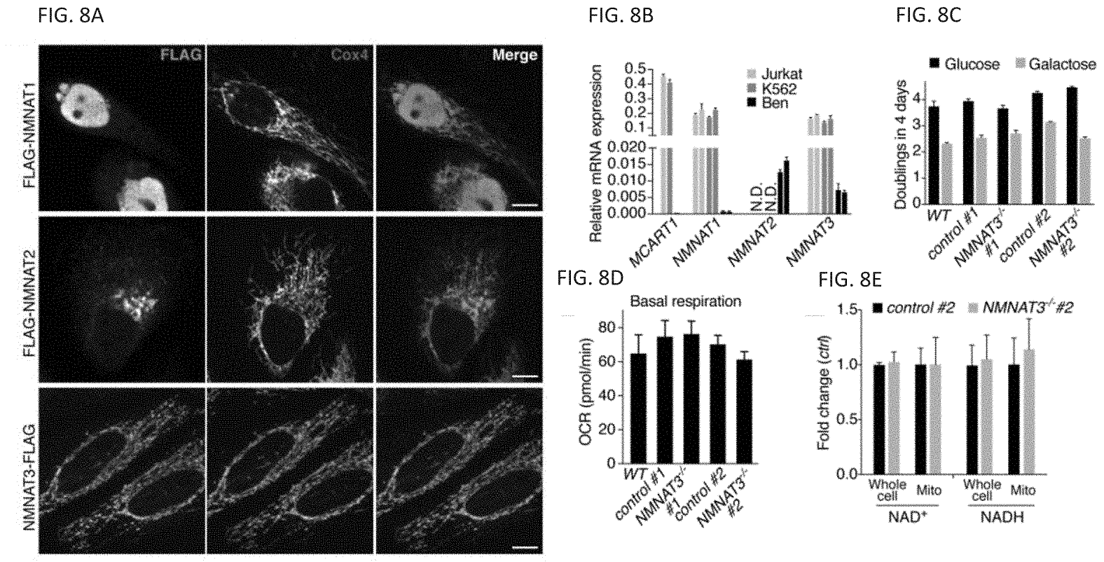

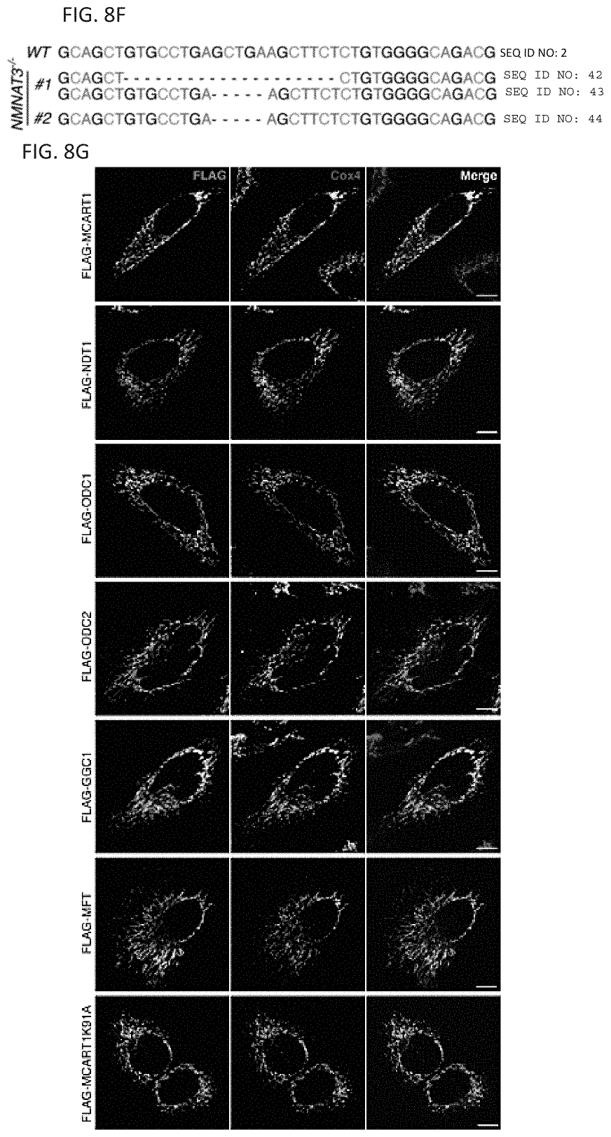

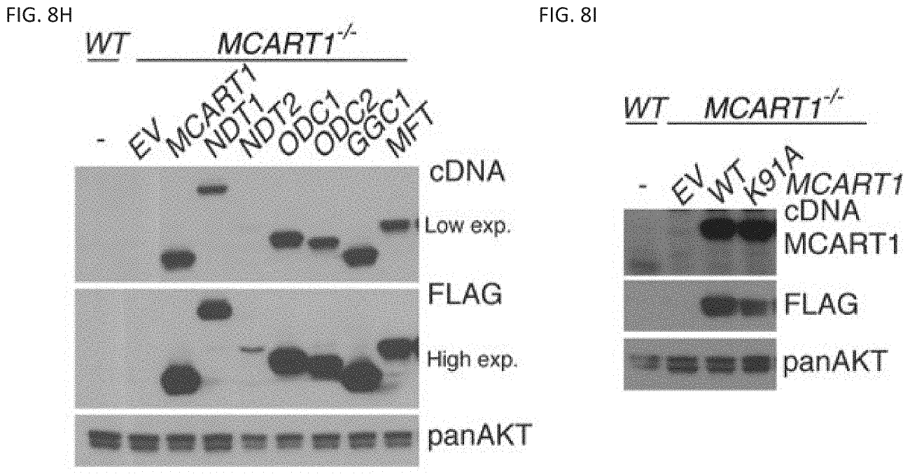

In mammals, de novo synthesis of NAD from tryptophan occurs primarily in the liver, while most other tissues rely on NAD synthesis or salvage from its precursors niacin, nicotinamide (Nam), nicotinamide riboside (NR) or nicotinamide mononucleotide (NMN) (27, 28). The precise step at which NAD is transported into mitochondria and whether NAD can be synthesized in mitochondria is unclear (FIG. 4A). Both NMN and NAD have been proposed to be transported into mitochondria of human cells (27, 29, 30). However, deletion of NMNAT3, the mitochondrial NAD synthesis isoenzyme that uses NMN to generate NAD, which is expressed in Jurkat cells, did not affect respiration or mitochondrial NAD levels making NMN as the transported species highly unlikely (FIG. 8A-8F).

While a mammalian mitochondrial NAD transporter has not been identified, NDT1 and NDT2 are well known as the mitochondrial NAD⁺ transporters in yeast and plants (5, 6, 31). Like MCART1, NDT1 and 2 are part of the SLC25 family of mitochondrial transporters, but they are not closely related to MCART1 in sequence or predicted structure and sequence analysis has failed to reveal the mitochondrial NAD transporter in higher eukaryotes (32). To test whether NDT1 and NDT2 could functionally complement loss of MCART1, as would be predicted if MCART1 is required for NAD transport into mitochondria, cDNAs for NDT1 or 2 codon-optimized for expression in human cells was expressed in MCART1-null cells. NDT1 and 2 but neither of the closest yeast MCART1 homologs predicted based on sequence or structure, the 2-oxodicarboxylate carriers ODC1 and 2 and the GTP/GDP carrier GGC1, rescued mitochondrial respiration in MCART1-null cells (FIG. 4B, FIGS. 8G and 8H). Importantly, NDT1 also rescued complex I activity in MCART1-null cells and mitochondrial NAD levels (FIGS. 4C and 4D). The lesser rescue ability of NDT2 is likely due to lower expression/protein stability and activity (FIG. 8H, (5)). Of note, overexpression of the closest homolog of NDT1 in humans, the mitochondrial FAD and folate transporter MFT/SLC25A32, which scored as selectively essential in MCART1-null cells in our CRISPR screen, did very slightly improve respiration in MCART1-null cells (FIG. 4B).

Based on sequence alignment and previously published mutational and structural analysis of the ADP/ATP carrier (33-36) the structure of MCART1 was modeled. Three residues conserved across species, lysine 91, arginine 182, and arginine 278 and located on the inside of the pore, were identified as potential substrate contacts (FIG. 4E). Mutation of the conserved lysine residue 91, which is part of transmembrane helix 2, to alanine, abolished the ability of MCART1 to rescue mitochondrial NAD levels and respiration (FIGS. 4F and 4G) without significantly affecting protein stability or localization (FIGS. 8G and 8I), indicating that MCART1 is likely a transport channel and not simply an auxiliary factor. Further research will reveal whether this is indeed the case and whether NAD⁺, NADH, or a related metabolite is its direct substrate.

Discussion

The results herein show that MCART1 is required for electron transport chain function by maintaining mitochondrial levels of NAD and NADH. Loss of MCART1 and mitochondrial NAD leads to diminished flux through pathways relying on NAD in mitochondria such as the TCA cycle, mitochondrial one-carbon metabolism, and oxidative phosphorylation. This work identifies for the first time a putative mitochondrial NAD importer in higher eukaryotes and thus addresses a long-standing question in the metabolism and mitochondrial biology fields.

Deletion of the mitochondrial isoform of the NAD synthesis enzyme NMNAT, NMNAT3, did not affect mitochondrial NAD levels, corroborating recent studies claiming NAD itself is the imported species and makes NMN as the substrate unlikely (30). Indeed, the yeast NAD⁺ transporter NDT1 is able to rescue mitochondrial NAD levels as well as complex I activity, implicating MCART1 as its functional homolog. MCART1 activity must not interfere with the malate-aspartate shuttle, which is responsible for exchanging reducing equivalents in the form of NADH across the inner mitochondrial membrane serving as an electron transport system for coupling NADH production by glycolysis in the cytosol to oxidative phosphorylation in mitochondria.

Despite displaying severe metabolic changes, and strong mitochondrial respiration and growth defects, MCART1-null cells are viable. Glycolysis provides ATP in MCART1-null cells and it is possibly that NAD-requiring reactions shift to other cellular compartments where isoenzymes are present to compensate for loss of the production of certain metabolites in mitochondria. It is also possible that another transporter is able to maintain basal levels of NAD in mitochondria in the absence of MCART1. Candidates are the mitochondrial FAD/folate transporter MFT/SLC25A32, which was synthetically lethal with MCART1 in our screen, as well as MCART2, although its expression was not detected. Determining how MCART1-null cells adapt and rewire their metabolism to loss of mitochondrial NAD could uncover metabolite or genetic interventions that are able to bypass MCART1 function and might be useful strategies to treat complex I deficiency. In this way, MCART1 deficiency could be useful as a model for mitochondrial disease.

NAD plays a critical role in cellular and mitochondrial metabolism beyond its role as an enzymatic co-factor in redox reaction as co-substrate of sirtuins and poly-ADP-ribose-polymerases. Three sirtuin homologs, SIRT3-5, serving as signaling factors connecting metabolism to cell state are present in mitochondria and their activity is likely coupled to mitochondrial NAD levels (31, 37). As NAD levels decline in aging and recent efforts are aimed at boosting cellular NAD levels to delay the onset of aging and age-related diseases (1, 2, 38), MCART1 emerges as a tool to study the role of the mitochondrial NAD pool in the regulation of these processes and as an interesting target to modulate life span.

Materials and Methods

Reagents

Reagents were obtained from the following sources: the antibodies that recognize SHMT2 (HPA020549) from Atlas Antibodies; AKT (4691), CALR (12238), Catalase (12980), Citrate Synthase (14309), Cytochrome c oxidase subunit 4 isoform 1 (COX4; 4850), GOLGA1 (13192), RPSS6KB1 (2708), VDAC (4661), the myc (2278) and HA epitopes (3724) and HRP-coupled anti-rabbit secondary antibody as well as Normal Donkey Serum from Cell Signaling Technology (CST); the FLAG epitope from CST (2368) and Sigma (F1804); LAMP2 (sc-18822), TOM20 (sc-11415) and HRP-labeled anti-mouse secondary from Santa Cruz Biotechnology (SCBT); MCART1/SLC25A51 (CSB-PA875649LA01HU) from Cusabio; total OXPHOS Rodent WB Antibody Cocktail (ab110413), ND6 (ab81212) and NDUFS3 (ab177471) from Abcam; Cytochrome c oxidase subunit 1 (COX1; 459600) from Invitrogen; Cytochrome c oxidase subunit 2 (COX2, MTCO2; A-6404) from Life Technologies and the ND1 (55410-1-AP) and ND5 (19703-1-AP) antibodies from Proteintech Group Inc. Antibodies against mitochondrially encoded proteins were validated using ρ⁰-cells. Amino acids, galactose, oligomycin, FCCP, rotenone, antimycin, sodium azide, pyruvic acid, malic acid, ascorbate, adenosine diphosphate, N,N,N′,N′-Tetramethyl-p-phenylenediamine, malonic acid and succinic acid were from Sigma Aldrich; duroquinol from TCI America; X-tremeGENE 9 and Complete Protease Cocktail from Roche; Alexa 488, 568, and 642-conjugated secondary antibodies and from Invitrogen; anti-HA magnetic beads from ThermoFisher Scientific; glucose from Westnet Inc. (# BM-675); ANTI-FLAG M2 Agarose Affinity Gel and sodium formate from Sigma; egg phosphatidylcholine, E. coli total lipids, and the lipid extruder from Avanti Polar Lipids; Bio-Beads SM-2 Adsorbents from Biorad Laboratories; filter membranes for extrusion and supports from Whatman; Cell-Tak from Corning.

Cell Lines and Plasmids

The pMXs-IRES-Bsd vector was from Cell Biolabs. The identities of the Jurkat, K562, and HeLa cells used in this study were authenticated by STR profiling. Jurkat cells were used for all functional studies in cells. Sequences of human MCART1, MCART2 and MCART6, MFT and S. cerevisiae NDT1 (YIL006W), NDT2 (YEL006W), ODC1 (YPL134C), ODC2 (YOR222W) and GGC1 (YDL198C) were synonymously mutated to remove the proto-spacer adjacent motif (PAM) sequence and/or codon-optimized for expression in human cells.

| Plasmid name | Addgene ID |
|---|---|
| pMXs_FLAG-MC ART 1 | 133247 |
| pMXs_FLAG-MC ART 1K91A | 133248 |
| pMXs_FLAG-MC ART 1R182A | 133252 |
| pMXs_MCARTl | 133253 |
| pMXs_FLAG-MCART2 | 133250 |
| pMXs_FLAG-MCART6 | 133251 |
| pMXs_FLAG-NDTl | 133254 |
| pMXs_FLAG-NDT2 | 133255 |
| pMXs_FLAG-ODCl | 133256 |
| pMXs_FLAG-ODC2 | 133257 |
| pMXs_FLAG-GGCl | 133258 |
| pMXs_FLAG-NMNAT 1 | 133259 |
| pMXs_FLAG-NMNAT2 | 133260 |
| pMXs_NMNAT3 -FLAG | 133261 |
| pMXs_FLAG-MFT | 136371 |

Cell Culture

Unless otherwise indicated, Jurkat and K562 cells were cultured in RPMI (Life Technologies) supplemented with 10% Inactivated Fetal Calf Serum (IFS, Sigma and Gemini), 2 mM glutamine, and penicillin/streptomycin. HeLa, HEK-293T, and Ben cells were cultured in DMEM (Life Technologies) supplemented with 10% IFS and penicillin/streptomycin. HEK-293T cells used for virus production were cultured in IMDM (Life Technologies) supplemented with 20% IFS, and penicillin/streptomycin. To compare proliferation of cells in glucose to proliferation in galactose, RPMI without glucose (Life Technologies) was supplemented with dialyzed IFS and either 10 mM glucose or galactose. All cell lines were maintained at 37° C. and 5% CO₂.

Virus Production

HEK-293T cells were co-transfected with the pLentiCRISPR sgRNA library, the VSV-G envelope plasmid and the AVPR lentiviral packaging plasmid, or with pMXS plasmids and retroviral packaging plasmids Gag-Pol and VSV-G, using X-TremeGene 9 Transfection Reagent. The culture medium was exchanged 24 hours after transfection with the same medium instead supplemented with 30% IFS. The virus-containing supernatant was collected 48 hours after transfection and spun for 5 min at 400×g to eliminate cells.

Transduction of Cell Lines

Cells were seeded at a density of 1×10⁶ cells/mL in RPMI containing 8 μg/mL polybrene (EMD Millipore), and then transduced with lentivirus by centrifugation at 2,200 RPM for 45 min at 37° C. After an 18-hour incubation, cells were pelleted to remove virus, washed twice in PBS and then re-seeded into fresh culture medium containing puromycin or blasticidin, and selected for 72 hours.

CRISPR/Cas9-Mediated Generation of Knockout Cell Lines

Human MCART1 and NMNAT3 were depleted using the pX330 system and the following sense (S) and antisense (AS) oligonucleotides:

sgMCART1_3 (S):
(SEQ ID NO: 12)
caccgGAGATGAAGCATTACTTGTG
sgMCART1_3 (AS):
(SEQ ID NO: 13)
aaacCACAAGTAATGCTTCATCTCc
sgNMNAT3_9 (S):
(SEQ ID NO: 14)
caccgCCACAGAGAAGCTTCAGCTC
sgNMNAT3_9 (AS):
(SEQ ID NO: 15)
aaacGAGCTGAAGCTTCTCTGTGGc

1 million Jurkat cells were electroporated with the 2.5 μg of sgRNA plasmid and GFP control plasmid at a 10:1 ratio using an Amaxa Cell Line Nucleofector Kit V and an Amaxa™ Nucleofector™ II (Lonza) and GFP-positive cells were single-cell FACS-sorted into 96-well plates. Cell clones with MCART1 knockouts were identified by western blotting and confirmed by next generation sequencing at the MGH CCIB DNA core. Two different knockout clones (designated #1 and #2) were used for experiments. Where not indicated, clone #1 was used. NMNAT3 knockout clones were identified by next generation sequencing.

CRISPR-Cas9 Synthetic Lethality Negative Selection Genetic Screen

MCART1-null or control cells re-expressing the guide-resistant MCART1 cDNA were transduced with a genome-wide sgRNA library (7, 40). 48 hours after infection, cells were selected with puromycin for 72 hours. Subsequently, cells were passaged every other day at a seeding density of 250,000 cells/ml until reaching ˜14 population doublings (PDs). DNA was extracted from 80×10⁶ cells using the QIAamp DNA Blood Maxi Kit (QIAGEN). sgRNA inserts were PCR amplified using ExTaq DNA Polymerase (Takara). The resultant PCR products were purified and sequenced on a HiSeq 2500 (Illumina) (primer sequences provided below) to monitor the change in the abundance of each sgRNA between the initial and final cell populations.

Primer sequences for sgRNA quantification

Forward:
(SEQ ID NO: 16)
AATGATACGGCGACCACCGAGATCTACACGAATACTGCCATTTGTCTCAA
GATCTA
Reverse:
(SEQ ID NO: 17)
CAAGCAGAAGACGGCATACGAGATCnnnnnnTTTCTTGGGTAGTTTGCAG
TTTT (nnnnnn denotes the sample barcode).
Illumina sequencing primer
(SEQ ID NO: 18)
CGGTGCCACTTTTTCAAGTTGATAACGGACTAGCCTTATTTTAACTTGCT
ATTTCTAGCTCTAAAAC
Illumina indexing primer
(SEQ ID NO: 19)
TTTCAAGTTACGGTAAGCATATGATAGTCCATTTTAAAACATAATTTTAA
AACTGCAAACTACCCAAGAAA

Sequencing reads were aligned to the sgRNA library and the abundance of each sgRNA was calculated. sgRNAs with less than 50 counts in the initial cell pool were removed from downstream analyses. The log₂fold-change in abundance of each sgRNA was calculated for each treatment condition after adding a pseudocount of one. A pseudocount of one was added to all sgRNAs and counts were normalized by number of reads in each sample multiplied by one million. Gene scores were defined as the average log2fold-change in the abundance of all sgRNAs targeting a given gene between the initial and final cell populations and calculated for both cell lines and Z-score normalized. The differential gene score was calculated as the difference in gene scores between cell lines.

Cell Proliferation Assays

10,000 cells per well were seeded into 96-well plates in triplicate. Cell titer glo reagent (Promega) was added to one plate 1 hour after seeding and luminescence was measured, while a second plate was read-out 4 days after seeding. Number of doublings in 4 days was determined by calculating the logₑ fold-change in signal between day 0 and 4. For cell counting experiments, 100,000 cells/ml were seeded in a 6-well plate in triplicates and counted immediately after seeding and from then on every 24 hours for 5 sequential days. For metabolite rescue experiments, 1 mM pyruvate and 100 μg/ml uridine, 1 mM formate or 0.1 mM/16 μM hypoxanthine/thymidine were added at the beginning of the culture period.

mRNA Quantification by qPCR

Genomic DNA was extracted from cells using the QIAamp DNA mini kit according to the manufacturer's instructions (Qiagen) and RNA reverse transcribed using the SuperScript III Reverse Transcriptase (Thermo Fisher Scientific). The following primers were used to assess mRNA levels of MCART1, NMNAT1 , NMNAT2 and NMNAT3 by qPCR by normalizing Ct values to those of β-ACTIN. MCART1_L:

MCARTL_R:
(SEQ ID NO: 20)
TAAGGAGCATCTGCCTACCG;
NMNAT1_L1:
(SEQ ID NO: 21)
CCCAACATGGCACCCAATAG;
NMNAT1_R1:
(SEQ ID NO: 22)
AAGCTGTGCCAAAGGTCAAG;
NMNAT1_L2:
(SEQ ID NO: 23)
TTCCAGCCCGAGTAACACAT;
NMNAT1_R2:
(SEQ ID NO: 24)
GTGGTTCTCCTTGCTTGTGG;
NMNAT2_L1:
(SEQ ID NO: 25)
TAGTCCTTGGCCAGCTCAAA;
NMNAT2_R1:
(SEQ ID NO: 26)
GTGGAGCGTTTCACCTTTGT;
NMNAT2_L2:
(SEQ ID NO: 27)
CACCTCCATATCTGCCTCGT;
NMNAT2_R2:
(SEQ ID NO: 28)
CCGTCTCATCATGTGTCAGC;
NMNAT3_L1:
(SEQ ID NO: 29)
AGGTGTCATGGAAGGTGTGT;
NMNAT3_R1:
(SEQ ID NO: 30)
GATGCGCACATCCAGGAAAT;
NMNAT3_L2:
(SEQ ID NO: 31)
TTGGCCAGGTGAATGTTGTG;
NMNAT3_R2:
(SEQ ID NO: 32)
ATGGGAAGAAAGACCTCGCA;
β-ACTIN_L:
(SEQ ID NO: 33)
CCTCAGCACCTTCACTGTCT;
β-ACTIN_R:
(SEQ ID NO: 34)
AGGATGGCAAGGGACTTCCTG;
(SEQ ID NO: 35)
AATGTGGCCGAGGACTTTGAT.

Immunofluorescence Assays and STED Imaging

For immunofluorescence assays 50,000 HeLa cells were plated in a 24-well glass bottom imaging plate (Cellvis, Mountain View, Calif.) and transfected with 500 ng of the cDNAs for FLAG constructs 16 hours later. 48 hours after transfection, cells were rinsed twice with PBS and fixed with 3% paraformaldehyde with 0.1% glutaraldehyde in PBS for 10 minutes. The fixation and all subsequent steps were performed at room temperature. Cells were rinsed three times with PBS and permeabilized with 0.3% NP40, 0.05% Triton X-100, 0.1% BSA in PBS for 3 minutes. After rinsing three times with wash buffer (0.05% NP40, 0.05% Triton-X 100, 0.2% BSA in PBS) samples were blocked for 1 hour in blocking buffer (0.05% NP40, 0.05% Triton-X 100, 5% Normal Donkey Serum). The samples were incubated with primary antibody in blocking buffer for 1 hour, washed three times with wash buffer, and incubated with secondary antibodies produced in donkey (diluted 1:500 in blocking buffer) for 30 minutes in the dark, washed three times with wash buffer, and rinsed three times with PBS. The primary antibodies used were directed against COX4 (CST; 1:250 dilution), the FLAG epitope (Sigma, 1:500 dilution) and TOM20 (SCBT, 1:500). Secondaries antibodies conjugated with Alexa 488 and 568 were used for confocal microscopy. Images were acquired on a Zeiss AxioVert200M microscope with a 63X oil immersion objective and a Yokogawa CSU-22 spinning disk confocal head with a Borealis modification (Spectral Applied Research/Andor) and a Hamamatsu ORCA-ER CCD camera. The MetaMorph software package (Molecular Devices) was used to control the hardware and image acquisition. The excitation lasers used to capture the images were 488 nm and 561 nm. Images were processed with FIJI (41). STED imaging was carried out on a Leica TCS SP8 STED 3X setup with an HC PL APO 100×/1.40 oil STEDwhite objective. Samples were fixed as described above. FLAG was detected using Alexa 594 secondary antibodies, COX4 with Atto647N (Sigma Aldrich), and Tom20 with Alexa 488. 660 nm and 775 nm or 592 nm depletion lasers were used. Images were deconvolved using the Adaptive Lightning strategy (Leica). Line profiles were generated from the raw data using FIJI.

MS-Based Metabolomics and Quantification of Metabolite Abundances

Metabolite abundance using LC/MS-based metabolomics was measured and quantified as previously described (10). Briefly, Jurkat cells were seeded at a density of 0.6×10⁶ per ml. 24 hours later, 1.5-2×10⁶cells were harvested, washed once in ice-cold 0.9% saline prepared with LC-MS-grade water, and extracted with 80% methanol containing 500 nM isotope-labeled amino acids as internal standards (Cambridge Isotope Laboratories). The samples were vortexed for 10 min at 4° C. and centrifuged at 17,000×g. The supernatant was dried by vacuum centrifugation at 4° C. Samples were stored at −80° C. until analyzed. On the day of analysis, samples were resuspended in 50-100 μL of LC-MS-grade water and the insoluble fraction was cleared by centrifugation at 15,000 rpm. The supernatant was then analyzed as previously described by LC-MS (10, 20). Amino acids were normalized to their respective internal standards, TCA cycle intermediates, malate, carnitine and C5-carnitine were normalized to the glutamate internal standard.

Glucose Consumption and Metabolite Secretion Assay

For media metabolite extraction, 6 wells of a 6-well plate were seeded with 300,000 Jurkat cells in 2 ml RPMI. The next day, cells were washed in PBS and resuspended in media. 1 ml of the upper three wells was collected and centrifuged at 3000 RPM for 5 min. to pellet cells. For the remaining media, the number of cells was counted. Two days later, the same procedure was repeated for the lower three wells. Metabolites were extracted from the media with a 75/25/0.2 extraction mix acetonitrile/methanol/formic acid with internal standards by vortexing for 10 min at 4° C. Samples were centrifuged at 17,000×g and the supernatant was analyzed by LC/MS. To calculate metabolite secretion or consumption rates, the difference in concentration between day 2 and day 0 were divided by the area under the growth curve according to (42).

Mitochondrial Isolations for Immunoblot Analyses

30×10⁶ Jurkat cells expressing the HA-mito tag or a control tag were washed 1×in PBS, 1× in KPBS according to (20). 5 μl of the cell suspension in 1 ml KPBS was lysed in 50 μl of 1% Triton lysis buffer to obtain whole cell protein levels. The rest was lysed using 8 strokes with a 30 ½ G needle. Lysates were spun for 1 min at 1000×g to pellet unbroken cells, and subsequently incubated with 100 μl HA-magnetic beads for 4 min. Beads were washed 3× in KPBS, and mitochondria lysed in 50 μl lysis buffer for 10 min. Beads were removed using the magnet, and samples were spun 10 min at 17,000×g to remove residual beads and insoluble material. SDS-PAGE loading dye was added to each sample, and 6 μl of whole cell lysate and 9 μl of the mitochondrial fraction were analyzed by SDS-PAGE.

Mitochondrial Isolations for Metabolite Analyses

Mitochondria were isolated using the Mito-IP method as described above for immunoblotting, except cells were disrupted with 20 strokes in a homogenizer containing a pure PTFE head (VWR International) and two strokes with a dounce tissue grinder with tight-fitting pestle (DWK Life Sciences Kimbl Kontes). 850 μl of the final suspension of beads with bound mitochondria in 1 ml KPBS were used for metabolite extraction and the remaining 150 μl for immunoblotting to determine mitochondrial capture efficiency. Metabolites were extracted with 50 μl 80% methanol containing internal standards. To obtain paired whole cell metabolite quantification, 25 μl of the initial cell suspension in 1 ml KPBS were extracted in 225 μl of 80% methanol with internal standards. Where indicated an extraction buffer consisting of 2:2:1 acetonitrile:methanol:water was used to preserve NAD⁺ and NADH followed by quenching with 15% (w/v) ammonium bicarbonate (8.7 μl per 100 μl solvent; adapted from (21)). 5 μl of the mitochondrial extract was injected for mass spectrometry analysis. Mitochondrial metabolite levels were normalized based on the citrate synthase signal determined by western blot.

Glutamine Tracing Experiments

For glutamine tracing experiments in whole cells, cells were incubated in RPMI containing 2 mM ¹³C₅,¹⁵N₂-glutamine (Cambridge Isotope Labs) as the sole glutamine source for 2 hours before metabolites were extracted and quantified as described above with the exception that no internal standards were added to the extraction buffer. To measure TCA cycle flux in isolated mitochondria, mitochondria were purified by Mito-IP as described above. Mitochondria bound to magnetic beads were incubated in 100 μl KPBS containing 4 mM ¹³C₅, ¹⁵N₂-glutamine, 0.5 mM malate, 1 mM ADP at 33° C. for 2.3 hours with rocking. The reaction was stopped and metabolites extracted by addition of 150 μl ice-cold acetonitrile. Samples were vortexed and centrifuged at 17,000 ×g for 10min. 4 μl of a 1:10 dilution in 80% methanol was injected for mass spectrometric detection. Metabolites were normalized based on the citrate synthase signal determined by western blot.

Seahorse Extracellular Flux Analyses

Oxygen consumption rates (OCR) of intact cells were measured using an XFe96 Extracellular Flux Analyzer (Agilent). 100,000 Jurkat cells were seeded on Seahorse XFe96 culture plates coated with Cell-tak and assayed after incubation at 37° C. for 1 h. Three basal OCR measurements were taken, followed by sequential injections of 1 μM oligomycin, 3 μM FCCP, and 1 μM antimycin A, taking three measurements following each treatment. Cellular respiration was calculated by subtracting the OCR after Antimycin A treatment from the basal or FCCP-stimulated OCR. ATP synthesis was measured with the Seahorse XF Real-Time ATP Rate Assay kit (Agilent) according to the manufacturer's instructions. Activity measurements for each respiratory chain complex in permeabilized cells were performed according to (43). Briefly, after seeding of cells the media was changed to Mannitol and sucrose (MAS)-BSA buffer (70 mM sucrose, 220 mM Mannitol, 10 mM KH₂PO₄, 5 mM MgCl₂, 2 mM HEPES pH 7.2, 1 mM EGTA, 0.4% BSA) and flux measurements were started. After three basal measurements, cells were permeabilized by injection of XF Plasma Membrane Permeabilizer (Agilent; 1 nM final concentration) together with 1 mM ADP and the following respiratory complex substrates or ADP only: complex I—pyruvate/malate (5 mM/2.5 mM); complex II—succinate/rotenone (10 mM/1 μM); complex III—duroquinol (0.5 mM); complex IV—N,N,N,N-tetramethyl-p-phenylenediamine/ascorbate (0.5 mM/2 mM final concentration). These were followed by injections with Oligomycin (1 μg/ml final), and respective complex inhibitors (complex I—1 μM rotenone, complex II,III—20 μM antimycin A, complex IV—20 mM sodium azide).

Characterization of Mitochondria

For quantification of mitochondrial mass and morphology, Jurkat cells were stained with MitoTracker Green (Life Technologies, M22426) at 25 nM for 1 h before analysis by flow cytometry or fluorescence microscopy. For microscopy, nuclei were stained with Hoechst 33342 fluorescent stain (Molecular Probes) at 2 μg/ml and z-stacks with 250 nm step size were taken at 100× magnification. FIJI was used to generate max intensity z-projections and measure mitochondrial length (41). To measure mitochondrial membrane potential cells were stained with 200 nM tetramethylrhodamine, methyl ester, perchlorate (TMRM; Life Technologies, T668) in RPMI for 20 min at 37° C., washed once with PBS, and resuspended in fresh PBS for flow cytometry analysis of live cells. Where indicated, cells were incubated with 10 μM FCCP for 10 min prior to adding TMRM dye. For ultrastructural analysis by electron microscopy, cells were were fixed in 2.5% gluteraldehyde, 3% paraformaldehyde with 5% sucrose in 0.1 M sodium cacodylate buffer (pH 7.4), pelletted, and post fixed in 1% OsO4 in veronal-acetate buffer. The cells were stained en block overnight with 0.5% uranyl acetate in veronal-acetate buffer (pH6.0), then dehydrated and embedded in Embed-812 resin. Sections were cut on a Leica EM UC7 ultra microtome with a Diatome diamond knife at a thickness setting of 50 nm, stained with 2% uranyl acetate, and lead citrate. The sections were examined using a FEI Tecnai spirit at 80 KV and photographed with an AMT ccd camera. Analysis of mtDNA copy number was performed as previously described (12). Briefly, genomic and mitochondrial DNA were extracted from cells using the QIAamp DNA mini kit according to the manufacturer's instructions (Qiagen). The following primers targeting the mitochondrial gene ND1 and the nuclear gene RUNX2 were used to assess mtDNA copy number by qPCR by normalizing Ct values of ND1 to those of RUNX2. ND1_LF: CCC TAA AAC CCG CCA CAT CT (SEQ ID NO: 36); ND1_R: GAG CGA TGG TGA GAG CTA AGG T (SEQ ID NO: 37); RUNX2_F: CGC ATT CCT CAT CCC AGT ATG (SEQ ID NO: 38); RUNX2_R: AAA GGA CTT GGT GCA GAG TTC AG (SEQ ID NO: 39). Jurkat whole cell lysates for immunoblot analysis of mitochondrial (and other) proteins were prepared by lysis in 1% Triton lysis buffer.

Bioinformatics Analyses

MCART1 (Q9H1U9; S2551_HUMAN) topology was predicted using Protter (9). The Broad Institute Achilles CRISPR data set (8) was analyzed in excel using the in-build correlation function to calculate MCART1 correlation with all genes in the dataset. In addition, the Achilles data was further analyzed in “R” (version 3.3.3, ×64) using custom written scripts. The limma package was used to generate barcode plots. Gene Ontology (GO) terms used for barcode plots are ETC: GO:0022900; TCA cycle: GO:0006099; Mitochondrial DNA replication: GO:0006264; Mitophagy: GO:0000423; Mitochondrial fusion: GO:0008053; Mitochondrial transmembrane transport: GO:1990542; Fatty acid beta-oxidation: GO:0006635. All groups were filtered for human genes only. For construction of the phylogenetic tree, protein sequences of all members of the human SLC25 family (obtained from (44)) were aligned using MUSCLE (45). The PHYLIP proml module (46) was used to construct the phylogenetic tree and FigTree software v.1.4.3 to visualize it. Percent sequence identities and similarities were calculated with the NCBI blastp tool. Graphpad Prism 7 software was used to generate the heat map of MCART RNA expression based on data from the Cancer Cell Line Encyclopedia (broadinstitute.org/ccle). MCART TPM (Transcripts Per Kilobase Million) levels in normal tissues were extracted from GTEx Portal V7. The MCART1 structure was modeled using Memoir membrane protein modelling pipeline (47) based on the structure of the bovine mitochondrial ADP/ATP carrier in complex with carboxyatractyloside ((33);PDB #1OKC). The high-coverage model was visualized using Chimera (48).

Statistical Analyses

Two-tailed t tests were used for comparison between two groups. All comparisons were two-sided, and P values of less than 0.05 were considered to indicate statistical significance. All error bars denote standard deviations between biological replicates unless indicated otherwise.

### REFERENCES

1. R. H. Houtkooper, C. Canto, R. J. Wanders, J. Auwerx, The secret life of NAD+: an old metabolite controlling new metabolic signaling pathways. Endocr Rev 31, 194-223 (2010).

2. E. Verdin, NAD(+) in aging, metabolism, and neurodegeneration. Science 350, 1208-1213 (2015).

3. L. Rajman, K. Chwalek, D. A. Sinclair, Therapeutic Potential of NAD-Boosting Molecules: The In Vivo Evidence. Cell Metab 27, 529-547 (2018).

4. L. R. Stein, S. Imai, The dynamic regulation of NAD metabolism in mitochondria. Trends in endocrinology and metabolism: TEM 23, 420-428 (2012).

5. S. Todisco, G. Agrimi, A. Castegna, F. Palmieri, Identification of the mitochondrial NAD+ transporter in Saccharomyces cerevisiae. The Journal of biological chemistry 281, 1524-1531 (2006).

6. F. Palmieri et al., Molecular Identification and Functional Characterization of Arabidopsis thaliana Mitochondrial and Chloroplastic NAD(+) Carrier Proteins. Journal of Biological Chemistry 284, 31249-31259 (2009).

7. T. Wang et al., Gene Essentiality Profiling Reveals Gene Networks and Synthetic Lethal Interactions with Oncogenic Ras. Cell 168, 890-903 e815 (2017).

8. R. M. Meyers et al., Computational correction of copy number effect improves specificity of CRISPR-Cas9 essentiality screens in cancer cells. Nature genetics 49, 1779-1784 (2017).

9. U. Omasits, C. H. Ahrens, S. Muller, B. Wollscheid, Protter: interactive protein feature visualization and integration with experimental proteomic data. Bioinformatics 30, 884-886 (2014).

10. K. Birsoy, Wang, T., Chen, W. W., Freinkman, E., Abu-Remaileh, M., Sabatini, D. M., An Essential Role of the Mitochondrial Electron Transport Chain in Cell Proliferation Is to Enable Aspartate Synthesis. Cell 162, 540-551 (2015).

11. M. P. King, G. Attardi, Human cells lacking mtDNA: repopulation with exogenous mitochondria by complementation. Science 246, 500-503 (1989).

12. N. Kory et al., SFXN1 is a mitochondrial serine transporter required for one-carbon metabolism. Science 362, eaat9528 (2018).

13. G. S. Ducker et al., Reversal of Cytosolic One-Carbon Flux Compensates for Loss of the Mitochondrial Folate Pathway. Cell Metab 23, 1140-1153 (2016).

14. H. Patel, E. D. Pietro, R. E. MacKenzie, Mammalian fibroblasts lacking mitochondrial NAD+-dependent methylenetetrahydrofolate dehydrogenase-cyclohydrolase are glycine auxotrophs. The Journal of biological chemistry 278, 19436-19441 (2003).

15. X. R. Bao et al., Mitochondrial dysfunction remodels one-carbon metabolism in human cells. Elife 5, (2016).

16. I. Martínez-Reyes et al., TCA cycle and mitochondrial membrane potential are necessary for diverse biological functions. Molecular cell 61, 199-209 (2016).

17. A. R. Grassian et al., IDH1 mutations alter citric acid cycle metabolism and increase dependence on oxidative mitochondrial metabolism. Cancer Res 74, 3317-3331 (2014).

18. C. A. Lewis et al., Tracing compartmentalized NADPH metabolism in the cytosol and mitochondria of mammalian cells. Molecular cell 55, 253-263 (2014).

19. R. P. Hausinger, FeII/alpha-ketoglutarate-dependent hydroxylases and related enzymes. Critical reviews in biochemistry and molecular biology 39, 21-68 (2004).

20. W. W. Chen, E. Freinkman, T. Wang, K. Birsoy, D. M. Sabatini, Absolute Quantification of Matrix Metabolites Reveals the Dynamics of Mitochondrial Metabolism. Cell 166, 1324-1337 e1311 (2016).

21. W. Lu, L. Wang, L. Chen, S. Hui, J. D. Rabinowitz, Extraction and Quantitation of Nicotinamide Adenine Dinucleotide Redox Cofactors. Antioxid Redox Signal 28, 167-179 (2018).

22. D. Hellebrekers et al., Novel SLC25A32 mutation in a patient with a severe neuromuscular phenotype. Eur J Hum Genet 25, 886-888 (2017).

23. M. Schiff et al., SLC25A32 Mutations and Riboflavin-Responsive Exercise Intolerance. The New England journal of medicine 374, 795-797 (2016).

24. E. A. McCarthy, S. A. Titus, S. M. Taylor, C. Jackson-Cook, R. G. Moran, A mutation inactivating the mitochondrial inner membrane folate transporter creates a glycine requirement for survival of chinese hamster cells. The Journal of biological chemistry 279, 33829-33836 (2004).

25. S. A. Titus, R. G. Moran, Retrovirally mediated complementation of the glyB phenotype—Cloning of a human gene encoding the carrier for entry of folates into mitochondria. Journal of Biological Chemistry 275, 36811-36817 (2000).

26. A. N. Spam et al., Identification of the human mitochondrial FAD transporter and its potential role in multiple acyl-CoA dehydrogenase deficiency. Mol Genet Metab 86, 441-447 (2005).

27. A. Nikiforov, C. Dolle, M. Niere, M. Ziegler, Pathways and subcellular compartmentation of NAD biosynthesis in human cells: from entry of extracellular precursors to mitochondrial NAD generation. The Journal of biological chemistry 286, 21767-21778 (2011).

28. L. Liu et al., Quantitative Analysis of NAD Synthesis-Breakdown Fluxes. Cell Metab 27, 1067-1080 e1065 (2018).

29. M. Barile, S. Passarella, G. Danese, E. Quagliariello, Rat liver mitochondria can synthesize nicotinamide adenine dinucleotide from nicotinamide mononucleotide and ATP via a putative matrix nicotinamide mononucleotide adenylyltransferase. Biochem Mol Biol Int 38, 297-306 (1996).

30. A. Davila et al., Nicotinamide adenine dinucleotide is transported into mammalian mitochondria. Elife 7, (2018).

31. I. Orlandi, G. Stamerra, M. Val, Altered Expression of Mitochondrial NAD(+) Carriers Influences Yeast Chronological Lifespan by Modulating Cytosolic and Mitochondrial Metabolism. Front Genet 9, 676 (2018).

32. M. R. VanLinden et al., Subcellular Distribution of NAD(+) between Cytosol and Mitochondria Determines the Metabolic Profile of Human Cells. Journal of Biological Chemistry 290, 27644-27659 (2015).

33. E. Pebay-Peyroula et al., Structure of mitochondrial ADP/ATP carrier in complex with carboxyatractyloside. Nature 426, 39-44 (2003).

34. J. J. Ruprecht et al., Structures of yeast mitochondrial ADP/ATP carriers support a domain-based alternating-access transport mechanism. Proceedings of the National Academy of Sciences of the United States of America 111, E426-434 (2014).

35. J. J. Ruprecht et al., The Molecular Mechanism of Transport by the Mitochondrial ADP/ATP Carrier. Cell 176, 435-447 e415 (2019).

36. D. R. Nelson, J. E. Lawson, M. Klingenberg, M. G. Douglas, Site-directed mutagenesis of the yeast mitochondrial ADP/ATP translocator. Six arginines and one lysine are essential. Journal of molecular biology 230, 1159-1170 (1993).

37. S. I. Imai, L. Guarente, It takes two to tango: NAD(+) and sirtuins in aging/longevity control. NPJ Aging Mech Dis 2, 16017 (2016).

38. C. R. Martens et al., Chronic nicotinamide riboside supplementation is well-tolerated and elevates NAD(+) in healthy middle-aged and older adults. Nature Communications 9, (2018).

39. J. Barretina et al., The Cancer Cell Line Encyclopedia enables predictive modelling of anticancer drug sensitivity. Nature 483, 603-607 (2012).

40. T. Wang et al., Identification and characterization of essential genes in the human genome. Science 350, 1096-1101 (2015).

41. J. Schindelin et al., Fiji: an open-source platform for biological-image analysis. Nat Methods 9, 676-682 (2012).

42. A. M. Hosios et al., Amino Acids Rather than Glucose Account for the Majority of Cell Mass in Proliferating Mammalian Cells. Developmental cell 36, 540-549 (2016).

43. J. K. Salabei, A. A. Gibb, B. G. Hill, Comprehensive measurement of respiratory activity in permeabilized cells using extracellular flux analysis. Nature Protocols 9, 421-438 (2014).

44. F. Palmieri, C. L. Pierri, A. De Grassi, A. Nunes-Nesi, A. R. Fernie, Evolution, structure and function of mitochondrial carriers: a review with new insights. Plant J 66, 161-181 (2011).

45. R. C. Edgar, MUSCLE: multiple sequence alignment with high accuracy and high throughput. Nucleic Acids Res 32, 1792-1797 (2004).

46. J. Felsenstein, PHYLIP—Phylogeny Inference Package (Version 3.2). Cladistics 5, 164-166 (1989).

47. J. P. Ebejer, J. R. Hill, S. Kelm, J. Shi, C. M. Deane, Memoir: template-based structure prediction for membrane proteins. Nucleic Acids Res 41, W379-383 (2013).

48. E. F. Pettersen et al., UCSF Chimera—a visualization system for exploratory research and analysis. J Comput Chem 25, 1605-1612 (2004).

1. A method of modulating or stabilizing a Nicotinamide Adenine Dinucleotide (NAD) level in a mitochondria in a cell, comprising modulating the expression of MCART1 or the activity of a gene product of MCART1 in the cell.

2. The method of claim 1, wherein the level of NAD is stabilized.

3. The method of claim 1, wherein the expression of MCART1 or the activity of a gene product of MCART1 is increased, thereby increasing the level of NAD in the mitochondria.

4. The method of claim 1, wherein the expression of MCART1 or the activity of a gene product of MCART1 is decreased, thereby decreasing the level of NAD in the mitochondria.

5. The method of claim 1, wherein the expression of MCART1 or the activity of a gene product of MCART1 is modulated by contacting the cell with an agent.

6. The method of claim 5, wherein the agent comprises a peptide, nucleic acid, or small molecule.

7. A method of treating or preventing a disease or disorder associated with an aberrant level of NAD in mitochondria of a subject, comprising administering to the subject an agent that modulates the expression of MCART1 or the activity of a gene product of MCART1.

8. The method of claim 7, wherein administration of the agent stabilizes the level of NAD in mitochondria of the subject.

9. The method of claim 7, wherein administration of the agent increases the level of NAD in mitochondria of the subject.

10. The method of claim 7, wherein administration of the agent decreases the level of NAD in mitochondria of the subject.

11. The method of claim 7, wherein the agent comprises a peptide, nucleic acid, or small molecule.

12. The methods of claim 7, wherein the disease or disorder is a mitochondrial disease or disorder, a metabolic disease or disorder, a cardiovascular disease or disorder, a muscular disease or disorder, a neurological disease or disorder, a disease or disorder associated with fatigue, or a disease or disorder associated with aging.

13.-24. (canceled)

25. A method of inhibiting the growth or viability of a cancer cell, comprising contacting the cancer cell with an agent that reduces the expression of MCART1 or the activity of a gene product of MCART1.

26. The method of claim 25, further comprising contacting the cancer cell with a second agent having anti-cancer activity.

27. The method of claim 26, wherein the second agent inhibits the expression or activity of complex I.

28. The method of claim 27, wherein the second agent is an amiloride, an amiloride derivative, or a biguanide derivative.

29. The method of claim 25, wherein the cancer cell is contacted in vivo.

30.-37. (canceled)
